(19) United States
(12) Patent Application Publication
(10) Pub. No.: US 2023/0270884 A1
(43) Pub. Date: Aug. 31, 2023

(54) COMPOSITIONS USEFUL FOR TREATMENT OF CHARCOT-MARIE-TOOTH DISEASE

(71) Applicant: The Trustees of the University of Pennsylvania, Philadelphia, PA (US)

(72) Inventors: James M. Wilson, Philadelphia, PA (US); Christian Hinderer, Baltimore, MD (US); Eileen Workman, Philadelphia, PA (US)

(21) Appl. No.: 18/005,504

(22) PCT Filed: Jul. 13, 2021
(86) PCT No.: PCT/US2021/041406

§ 371 Date: Jan. 13, 2023

Related U.S. Application Data
(60) Provisional application No. 63/173,045, filed on Apr. 9, 2021.
(60) Provisional application No. 63/051,336, filed on Jul. 13, 2020.

(51) Int. Cl. A61K 48/00; C12N 15/86; C12N 15/113; C12N 9/14; A61P 25/00

(52) U.S. Cl. CPC A61K 48/005; C12N 15/86; C12N 15/1137; C12N 9/14; A61P 25/00; C12Y 306/05

(57) ABSTRACT

Provided herein are rAAV and other vectors and compositions useful for treating a patient having CMT2 comprising: (a) a recombinant nucleic acid sequence encoding an engineered human mitofusin 2 coding sequence operably linked to regulatory sequences which direct expression thereof in a human target cell. Also provided are rAAV and other vectors and compositions useful for treating a patient having CMT2 comprising: (b) a nucleic acid sequence encoding at least one miRNA specific for an endogenous human mitofusin 2 sequence in a human CMT2A subject, wherein the miRNA coding sequence is operably linked to regulatory sequences which direct expression thereof in the subject. Further provided are compositions containing both the engineered hMfn2 coding sequence and the at least one miRNA coding sequence, wherein the engineered human mitofusin 2 coding sequence has a sequence which differs from endogenous human mitofusin 2 in the CMT2A patient in the target site of the encoded miRNA.

### BACKGROUND OF THE INVENTION

Charcot-Marie-Tooth (CMT) neuropathy is a heterogeneous group of inherited diseases found in peripheral nerves. CMT is a common disorder affecting both children and adults. Charcot-Marie Tooth disease (CMT) or hereditary motor and sensory neuropathy (HMSN) are the most commonly used names for inherited neuropathies that are not part of a syndrome (Klein, C. J., Duan, X., Shy, M. E., 2013. Inherited neuropathies: Clinical overview and update. Muscle Nerve; Bassam, B., 2014. Charcot-Marie-Tooth Disease Variants—Classification, Clinical, and Genetic Features and Rational Diagnostic Evaluation. J. Clin. Neuromusc. Dis. 15, 117-128; Scherer, S. S., Shy, M. E., 2015. CMT Subtypes and Disease Burden in Patients Enrolled in the INC Natural History Study (6601) from 2009-2013. J. Neurol. Neurosurg. Psychiat. 86, 873-878).

The dominantly inherited axonal forms of CMT neuropathy (type 2 CMT disorders) have normal or slightly reduced velocities with reduced motor and sensory compound action potential amplitudes, and axonal loss is the chief finding in biopsied nerves. Dominant mutations in the Mitofusin2 (MFN2) gene, a protein required for mitochondrial fusion and transport along axons, cause CMT2A (Zuchner, S., et al., 2004. Mutations in the mitochondrial GTPase mitofusin 2 cause Charcot-Marie-Tooth neuropathy type 2A. Nat Genet 36, 449-451). CMT2A is estimated to cause up to 7% of all CMT, and is the most common form of type 2 CMT (Freidman, V., et al., 2015. CMT Subtypes and Disease Burden in Patients Enrolled in the INC Natural History Study (6601) from 2009-2013. J. Neurol. Neurosurg. Psychiat. 86, 873-878). Different MFN2 mutations cause different degrees of neuropathy.

Most MFN2 mutations cause a severe, early onset, axonal neuropathy, and are de novo mutations. Other MFN2 dominant mutations cause a milder axonal neuropathy with a later onset (Lawson, V. H., et al, 2005. Clinical and electrophysiologic features of CMT2A with mutations in the mitofusin 2 gene. Neurology 65, 197-204; Chung, K. W., et al., 2006. Early onset severe and late-onset mild Charcot-Marie-Tooth disease with mitofusin 2 (MFN2) mutations. Brain 129, 2103-2118; Verhoeven, K., et al., 2006. MFN2 mutation distribution and genotype/phenotype correlation in Charcot-Marie-Tooth type 2. Brain 129, 2093-2102; Feely, S. M. E., et al., 2011, MFN2 mutations cause severe phenotypes in most patients with CMT2A. Neurology 76, 1690-1696). More rarely, recessive MFN2 mutations may cause severe, early onset, axonal neuropathy. Mfn2 mutations selectively toxic to lower motor neurons and primary sensory neurons in dorsal root ganglia (DRG).

Some dominant mutations in MFN2 produce myelopathy or optic atrophy; these complicated forms of CMT2 are sometimes referred to HMSN-V and HMSN-VI, respectively, but are not only caused by MFN2 mutations (Scherer, S. S., et al., 2015. Peripheral neuropathies in Rosenberg's Molecular and Genetic Basis of Neurological and Psychiatric Disease, 5th ed. Elsevier, Philadelphia, pp. 1051-1074).

Mammals have two mitofusin genes, Mfn1 and Mfn2, which have distinct but overlapping distributions, and both of which can promote mitochondrial fusion through trans-interactions (Chen, H., Chan, D. C., 2005. Emerging functions of mammalian mitochondrial fusion and fission. Hum. Mol. Genet. 14, R283-R289). Nearly all of the mutations in the MFN2 gene cause amino acid substitutions as single point mutations, including but not restricted to the GTPase domain (Cartoni, R., Martinou, J. C., 2009. Role of mitofusin 2 mutations in the physiopathology of Charcot-Marie-Tooth disease type 2A. Exp. Neurol. 218, 268-273). Because loss-of-function mutations of MFN2 also cause a severe axonal neuropathy (Nicholson, G. A., et al, 2008. Severe early-onset axonal neuropathy with homozygous and compound heterozygous MFN2 mutations. Neurology 70, 1678-1681), and result in reduced mitochondrial fusion (Chen, H., Chan, D. C., 2005. Emerging functions of mammalian mitochondrial fusion and fission. Hum. Mol. Genet. 14, R283-R289), it is possible that dominant MFN2 mutations have a dominant-negative effect on mitochondrial fusion (Detmer, S. A., Chan, D. C., 2007. Complementation between mouse Mfn1 and Mfn2 protects mitochondrial fusion defects caused by CMT2A disease mutations. J. Cell Biol. 176, 405-414: Baloh, R. H., et al., 2007. Altered axonal mitochondrial transport in the pathogenesis of Charcot-Marie-Tooth disease from mitofusin 2 mutations. J Neurosci 27, 422-430; Misko, A., et al., 2010. Mitofusin 2 is necessary for transport of axonal mitochondria and interacts with the Miro/Milton complex. J Neurosci 30, 4232-4240: Misko, A. L., et al., 2012. Mitofusin2 mutations disrupt axonal mitochondrial positioning and promote axon degeneration. J Neurosci 32, 4145-4155).

What are needed are treatments useful for reducing the symptoms, severity and/or progression of CMT2A and related disorders.

### SUMMARY OF THE INVENTION

Viral and non-viral vectors and compositions useful for treating patients having symptoms associated with defects in human mitofusin 2 expression and/or patients having CMT2A are provided herein.

In certain embodiments, a recombinant adeno-associated virus (rAAV) comprising an AAV capsid and a vector genome is provided. The rAAV comprises: (a) an engineered nucleic acid sequence encoding human mitofusin 2; (b) a spacer sequence located between (a) and (c); (c) a nucleic acid sequence encoding at least one miRNA sequence specific for endogenous human mitofusin 2 in a CMT2 patient located 3′ to the sequence of (a) and (b); wherein the engineered nucleic acid sequence of (a) lacks the target site for the encoded at least one miRNA, thereby preventing the encoded miRNA from targeting the engineered human mitofusin 2 coding sequence; and (c) regulatory sequences operably linked to (a) and (c). In certain embodiments, the AAV capsid is selected from AAV9, AAVhu68, AAV1 or AAVrh91. In certain embodiments, the spacer is 75 nucleotides to about 250 nucleotides in length. In one aspect, a vector is provided which comprises an engineered human mitofusin 2 coding sequence operably linked to regulatory sequences which direct expression thereof in a human target cell. In certain embodiments, a vector is provided which comprises a nucleic acid sequence encoding at least one hairpin miRNA, wherein the encoded miRNA is specific for endogenous human mitofusin 2 in a human subject operably linked to regulatory sequences which direct expression thereof in the subject. In certain embodiments, a vector or other composition comprises both the engineered human mitofusin 2 coding sequence and the at least one miRNA coding sequence. In such an embodiment, the engineered mitofusin 2 coding sequence lacks the target site for the at least one miRNA, thereby preventing the miRNA from targeting the engineered human mitofusin 2 coding sequence.

In certain embodiments, the vector is a replication-defective viral vector which comprises a vector genome comprising the human mitofusin 2 coding sequences, the coding sequence for the at least one miRNA and the regulatory sequences. In certain embodiments, the viral vector is a recombinant adeno-associated virus (rAAV) particle having an AAV capsid which has the packaged therein the vector genome. In certain embodiments, the AAV capsid is AAVhu68, AAV1 or AAVrh91.

In certain embodiments, a vector is provided which comprises a engineered mitofusin 2 coding sequence has the nucleic acid sequence of SEQ ID NO: 11 or a sequence at least 90% identical thereto, provided that the nucleic acid sequences targeted by the encoded miRNA are different from the endogenous human mitofusin 2 sequence.

In certain embodiments, a vector is provided which comprises a nucleic acid sequence comprising at least one miRNA coding sequence which comprises one or more of: (a) an miRNA coding sequence comprising SEQ ID NO 15 (miR1693, 64 nt); (b) an miRNA coding sequence comprising at least 60 consecutive nucleotides of SEQ ID NO: 15; (c) an miRNA coding sequence comprising at least 99% identity to SEQ ID NO: 15 which comprises a sequence with 100% identity to about nucleotide 6 to about nucleotide 26 of SEQ ID NO: 15 (or SEQ ID NO: 68); (d) an miRNA coding sequence comprising one or more of:

(i) TTGACGTCCAGAACCTGTTCT, SEQ ID NO: 27; (ii) AGAAGTGGGCACTTAGAGTTG, SEQ ID NO: 28; (iii) TTCAGAAGTGGGCACTTAGAG, SEQ ID NO: 29; (iv) TTGTCAATCCAGCTGTCCAGC, SEQ ID NO: 30; (v) CAAACTTGGTCTTCACTGCAG, SEQ ID NO: 31; (vi) AAACCTTGAGGACTACTGGAG, SEQ ID NO: 32; (vii) TAACCATGGAAACCATGAACT, SEQ ID NO: 33; (viii) ACAACAAGAATGCCCATGGAG, SEQ ID NO: 34; (ix) AAAGGTCCCAGACAGTTCCTG, SEQ ID NO: 35; (x) TGTTCATGGCGGCAATTTCCT, SEQ ID NO: 36; (xi) TGAGGTTGGCTATTGATTGAC, SEQ ID NO: 37; (xii) TTCTCACACAGTCAACACCTT, SEQ ID NO: 38; (xiii) TTTCCTCGCAGTAAACCTGCT, SEQ ID NO: 39; (xiv) AGAAATGGAACTCAATGTCTT, SEQ ID NO: 40; (xv) TGAACAGGACATCACCTGTGA, SEQ ID NO: 41; (xvi) AATACAAGCAGGTATGTGAAC, SEQ ID NO: 42; (xvii) TAAACCTGCTGCTCCCGAGCC, SEQ ID NO: 43; (xviii) TAGAGGAGGCCATAGAGCCCA, SEQ ID NO: 44; (xix) TCTACCCGCAGGAAGCAATTG, SEQ ID NO: 45; or (xx) CTCCTTAGCAGACACAAAGAA, SEQ ID NO: 46, or combinations of any of (i) through (xx). In certain embodiments, a single nucleic acid molecule comprises both the human mitofusin 2 coding sequence and the miRNA coding sequence and the nucleic acid molecule further comprises a spacer of at least 75 nucleotides between the hMfn2 coding sequence and the coding sequence at least one miRNA. In certain embodiments, the vector is a non-viral vector.

In certain embodiments, the composition comprises a recombinant nucleic acid sequence encoding an engineered human mitofusin 2 coding sequence operably linked to regulatory sequences which direct expression thereof in a human target cell and a nucleic acid sequence encoding at least one miRNA specific for endogenous human mitofusin 2 in a CMT2A patient operably linked to regulatory sequences which direct expression thereof in the subject, wherein the engineered mitofusin 2 coding sequence lacks a target site for the encoded at least one miRNA, thereby preventing the miRNA from targeting the engineered human mitofusin 2 coding sequence.

In certain embodiments, a pharmaceutical composition comprising the vector, rAAV, or a composition, and a pharmaceutically acceptable aqueous suspending liquid, excipient, and/or diluent.

In certain embodiments, a method for treating a patient having Charcot-Marie-Tooth (CMT) neuropathy is provided comprising delivering an effective amount of the vector, a recombinant AAV, or a composition to a patient in need thereof. In certain embodiments, a method for reducing neuropathy in a patient having Charcot-Marie-Tooth (CMT) neuropathy is provided which comprises delivering an effective amount of the vector, a recombinant AAV, or a composition to a patient in need thereof. In certain embodiments, a method for treating a patient is provided, wherein the method additionally comprising a combination with one or more co-therapies selected from: acetaminophen, nonsteroidal anti-inflammatory drugs (NSAIDs), tricyclic antidepressants or antiepileptic drugs, such as carbamazepine or gabapentin.

In certain embodiments, a combination regimen for treating a patient having CMT2A is provided which comprises co-administering (a) a recombinant nucleic acid sequence encoding an engineered human mitofusin 2 coding sequence operably linked to regulatory sequences which direct expression thereof in a human target cell, wherein the human mitofusin 2 coding sequence has the sequence of SEQ ID NO: 11 or a sequence at least 95% identical thereto and which differs from endogenous human mitofusin 2 in the CMT2A patient by having a mismatch in the miRNA target sequence of (b), (b) at least one miRNA specific for an endogenous human mitofusin 2 sequence in a human CMT2A subject, wherein the mRNA is operably linked to regulatory sequences which direct expression thereof in the subject.

In certain embodiments, a combination regimen for treating a patient having CMT2A is provided which comprises co-administering: (a) a recombinant nucleic acid sequence encoding an engineered human mitofusin 2 coding sequence operably linked to regulatory sequences which direct expression thereof in a human target cell, wherein the human mitofusin 2 coding sequence is engineered to differs from endogenous human mitofusin 2 in the CMT2A patient by having a mismatch in the miRNA target sequence of (b), (b) at least one miRNA specific for an endogenous human mitofusin 2 sequence in a human CMT2A subject, wherein the miRNA is operably linked to regulatory sequences which direct expression thereof in the subject. In certain embodiments, a first vector comprises the nucleic acid (a) and a second, different vector, comprises at least one miRNA (b). In certain embodiments, the first vector and/or the second vector are each a viral vector which may be same or different. In certain embodiments, the first and/or second vector are a non-viral vector.

These and other advantages will be apparent from the Detailed Description of the Invention which follows.

### BRIEF DESCRIPTION OF THE DRAWINGS

FIGS. 1A to 1B illustrate mitofusin 2 (Mfn2) miRNA selection (knockdown of endogenous Mfn2 with various miRNA). FIG. 1A illustrates knockdown of endogenous Mfn2 RNA, as measured by qPCR, in mouse brain in B6 mice following intravenous delivery of AAV-mediated delivery of miRNA. FIG. 1B illustrates knock down of endogenous mfn2 RNA, as measured by qPCR, in mouse spinal cord in B6 mice following intravenous delivery of AAV-mediated delivery of miRNA.

FIG. 2A to FIG. 2C illustrate Mfn2 RNA fold expression following delivery AAV vector comprising Mfn2 cDNA transgene (i.e., engineered nucleic acid sequence encoding Mfn2) and miR1518, wherein AAV vector was administered in mice intravenously at a dose of 3×10¹¹ GC. FIG. 2A illustrates mouse Mfn (mMfn2) RNA fold expression in spinal cord. FIG. 2B illustrates rat Mfn engineered (rMfn2co) RNA fold expression in spinal cord. FIG. 2C illustrates miR1518 RNA fold expression in spinal cord.

FIG. 3 illustrates plotted quantitation of western blot signal measuring percent expression of mitofurin-2 protein following miRNA treatment of B104 rat cells. Mitofurin-2 expression is plotted from calculated value of percent total over loading control of beta-actin.

FIG. 4 illustrates plotted quantitation of fold-expression of rat Mfn2 (rMfn2) cDNA expression in spinal cord of treated mice following AAV vector delivery of engineered rMfn2 cDNA transgene with miR1518.

FIG. 5 illustrates plotted quantitation of fold-expression of human Mfn2 (hMfn2) cDNA expression in spinal cord of treated mice following AAV vector delivery of engineered hMfn2 cDNA transgene with miR1693.

FIG. 6A and FIG. 6B illustrate total amount of mature miRNA processed from the AAV vectors following intravenous delivery in mice. FIG. 6A illustrates fold expression of miR1518 and miR1693, as measured by qPCR with miR1518 primers. FIG. 6B illustrates fold expression of miR1518 and miR1693, as measured by qPCR with miR1693 primers.

FIG. 7 shows expression levels of Mfn2 (Mfn2 expressed from vector) in Mfn2-null MEF cell line following transfection with various vectors comprising CB7 promoter; expression levels shown as plotted quantitation of western blot signal measuring expression of mitofurin-2 (Mfn2) following transfections with CB7.CI.hMfn2 GA.WPRE.RBG; CB7.CI.hMfn2.GA.LINK.miR1518.RBG; CB7.CI.hMfn2.GA.LINK.miR538.RBG.

FIG. 8 shows expression levels of Mfn2 (Mfn2 expressed from vector) in Mfn2-null MEF cell line following transfection with various vectors comprising CAG promoter; expression levels shown as plotted quantitation of western blot signal measuring expression of mitofurin-2 (Mfn2) following transfections with CAG.CI.hMfn2.GA.WPRE.SV40; CAG.CI.hMfn2.GA.LINK.miR1518.WPRE.SV40; CAG.CI.hMfn2.GA.LINK.miR538.WPRE.SV40. FIG.

FIG. 9A and FIG. 9B show expression levels of Mfn2 in HEK293 cell line following transfection with various vectors comprising either CB7 or CAG promoter. FIG. 9A show endogenous Mfn2 knockdown in HEK293 cells as measured by qPCR and plotted as fold expression, following transfection with various vectors comprising CB7 promoter, (CB7.CI.hMfn2.GA.WPRE.RBG; CB7. CI.hMfn2. GA.LINK.miR1518.RBG; CB7.CI.hMfn2.GA.LINK.miR538.RBG. FIG. 9B show endogenous Mfn2 knockdown in HEK293 cells as measured by qPCR and plotted as fold expression, following transfection with various vectors comprising a CAG promoter CAG.CI.hMfn2.GA.WPRE.SV40; CAG.CI.hMfn2.GA.LINK.miR1518.WPRE.SV40; CAG.CI.hMfn2 GA.LINK.miR538.WPRE.SV40.

FIG. 10 shows expression levels of Mfn2 (endogenous Mfn2 and Mfn2 expressed from vector) in HEK293 cell line following transfection with various vectors comprising CB7 promoter; expression levels shown as plotted quantitation of western blot signal measuring expression of mitofurin-2 (Mfn2) following transfections with CB7.CI.hMfn2.GA.WPRE.RBG; CB7.CI.hMfn2 GA.LINK.miR1518.RBG; CB7.CI.hMfn2.GA.LINK.miR538.RBG. Quantitation is plotted as percent expression; transfection efficiency was determined to be about 95%.

FIG. 11 shows expression levels of Mfn2 (endogenous Mfn2 and Mfn2 expressed from vector) in HEK293 cell line following transfection with various vectors comprising CAG promoter; expression levels shown as plotted quantitation of western blot signal measuring expression of mitofurin-2 (Mfn2) following transfections with CAG.CI.hMfn2 GA.WPRE.SV40 (p6168); CAG.CI.hMfn2.GA.LINK.miR1518.WPRE.SV40 (p6169); CAG.CI.hMfn2.GA.LINK.miR538.WPRE.SV40 (p6170). Quantitation is plotted as percent expression; transfection efficiency was determined to be about 95%.

FIG. 12A to FIG. 12C show expression levels, as measured by qPCR, of mature miRNA (miR1518 or miR538) in Mfn2-null MEF cell line (ATCC; CRL-2933), following transfection with various vectors comprising either CB7 or CAG promoter. FIG. 12A shows a comparison of expression levels, as measured by qPCR and plotted as fold expression, of mature miR1518 in Mfn2-null MEF cell line, following transfection with vectors comprising either CB7 or CAG promoter. FIG. 12B shows a comparison of expression levels, as measured by qPCR and plotted as fold expression, of mature miR538 in Mfn2-null MEF cell line, following transfection with vectors comprising either CB7 or CAG promoter. FIG. 12C shows a comparison of expression levels, as measured by qPCR and plotted as fold expression, of mature miR1518 and miR538 in Mfn2-null MEF cell line, following transfection with vectors comprising either CB7 or CAG promoter.

FIG. 13A to FIG. 13F show characterization of a mouse model. FIG. 13A show schematic representation of the mice genotype. FIG. 13B shows mice phenotype characterization, characterized by relative expression levels endogenous and FLAG-tagged MFN2 in brain as measured by western blotting. FIG. 13C shows mice phenotype characterization, characterized by relative expression levels endogenous and FLAG-tagged MFN2 in spinal cord as measured by western blotting. FIG. 13D shows measured weight in (g) of the mice in the CMT2A mouse model (nTg, MFN2^{WT}, and MFN2^{R49Q}). FIG. 13E shows mice phenotype characterization, as measured by the latency to fall (sec). FIG. 13F shows mice phenotype characterization, as measured by grip strength (g).

FIG. 14A and FIG. 14B show results of the pharmacological study in MFN2^{R94Q} mice (Study groups: G1—wild type (WT) mice, PBS; G2—MFN2^{R94Q} mice, PBS; G3—MFN2^{R94Q} mice, CB7.MFN2; G4—MFN2^{R94Q} mice, CB7.MFN2.miR1518; G5—MFN2^{R94Q} mice, CB7.MFN2.miR538; G6—MFN2^{R94Q} mice, CAG.MFN2.miR1518; G7—MFN2^{R94Q} mice, CAG.MFN2.miR538). FIG. 14A shows body weight results (plotted as (g)) as measured in mice groups G1 to G7. FIG. 14B shows survival results (plotted as probability of survival over day 0 to 50) as measured in mice groups G1 to G7.

FIG. 15 shows grip strength results (plotted as (kg)) of the pharmacological study in MFN2^{R94Q} mice.

### DETAILED DESCRIPTION OF THE INVENTION

Sequences, vectors and compositions are provided here for co-administering to a patient a nucleic acid sequence which expresses human mitofusin 2 (or hMfn2) protein and a nucleic acid sequence encoding at least one miRNA which specifically targets a site in the endogenous human mitofusin 2 gene of the patient which target site is not present on human mitofusin 2 engineered coding sequence. Suitably, the human mitofusin 2 coding sequence is engineered to remove the specific target site for the encoded miRNA. Novel engineered human mitofusin 2 coding sequences and novel miRNA sequences are provided herein. These may be used alone or in combination with each other and/or other therapeutics for the treatment of CMT2A.

As used herein the term “endogenous mitofusin 2” refers to the mitofusin 2 gene which encodes the mitofusin 2 protein in humans having CMT2A. Patients with CMT2A may have a number of missense mutations or allelic variants. See, also, omim.org/allelicVariants/608507, describing various allelic variants. Autosomal dominant Charcot-Marie-Tooth (CMT) disease type 2A2A (CMT2A2A) is caused by heterozygous mutation in the MFN2 gene (608507) on chromosome 1p36.2. Homozygous or compound heterozygous mutation in the MFN2 gene causes autosomal recessive CMT2A2B (617087), a more severe disorder with earlier onset. Another form of CMT2A mapping to chromosome 1p36.2, CMT2A1 (118210), is caused by mutation in the KIF1B gene (605995). See also hereditary motor and sensory neuropathy VI (HMSN6; 601152), an allelic disorder with overlapping features.

The native, functional, human Mfn2a gene, such as is found endogenously in patients without CMT2A, is reproduced in SEQ ID NO: 18 and the native, functional, human Mf2A protein is reproduced in SEQ ID NO: 19. Mitofusin 2 is made in many types of cells and tissues, including muscles, the spinal cord, and the nerves that connect the brain and spinal cord to muscles and to sensory cells that detect sensations such as touch, pain, heat, and sound (peripheral nerves). This gene may alternatively be called: CMT2A2, CRPP1, KIAA0214, MARF, MFN2 Human or mitochondrial assembly regulatory factor. See, OMIM.org/entry/609260, accessed Jul. 12, 2020. In certain embodiments, functional Mfn2 proteins having less than 100% identity to the amino acid sequence of SEQ ID NO: 19 may be delivered by the compositions provided herein (e.g., Mfn2 having 97% to 100% identity to SEQ ID NO: 19). In such embodiments, the enzymatic and binding function of native functional human Mfn2 are preferably retained. See, also, UniProtKB-09140 (e.g., binding sites at position 305 and 307 are conserved and/or nucleotide binding sites at nucleotides 106-111 and/or 258-261 are conserved).

In one embodiment, an engineered mitofusin 2 coding sequence is provided which has the nucleic acid sequence of SEQ ID NO: 11 or a sequence of about 90%, at least 95% identical, at least 97% identical, at least 98% identical, or 99% to 100% identical to SEQ ID NO: 11 and which expresses the human mitofusin 2 protein found in non-CMT2A patients. See, e.g., SEQ ID NO: 19. See, also SEQ ID NO: 2 and SEQ ID NO: 4.

In certain embodiments, an engineered mitofusin 2 coding sequence is provided which has the nucleic acid sequence of SEQ ID NO: 11 or a sequence at least 80% identical, provided that nt 216 to 236 of SEQ ID NO: 11 are conserved (e.g., 100% identical, or at least 99% identical), e.g., when the engineered coding sequence is co-administered with the miR538 coding sequence.

In certain embodiments, an engineered mitofusin 2 coding sequence is provided which has the nucleic acid sequence of SEQ ID NO: 11 or a sequence at least 80% identical, provided that nt 1371 to 1391 of SEQ ID NO: 11 are conserved (e.g., 100% identical, or at least 99% identical), e.g., when the engineered coding sequence is co-administered with the miR1518 coding sequence. Suitably, the sequence having identity to SEQ ID NO: 11 expresses the same protein. See, e.g., SEQ ID NO: 19; SEQ ID NO: 2 and SEQ ID NO: 4.

In one embodiment, an engineered mitofusin 2 coding sequence is provided which has the nucleic acid sequence of SEQ ID NO: 28 or a sequence at least 90%, at least 95% identical, at least 97% identical, at least 98% identical, at least 99% identical, and/or at least 99% to 100% identical to SEQ ID NO: 24.

A “5′ UTR” is upstream of the initiation codon for a gene product coding sequence. The 5′ UTR is generally shorter than the 3′ UTR. Generally, the 5′ UTR is about 3 nucleotides to about 200 nucleotides in length, but may optionally be longer.

A “3′ UTR” is downstream of the coding sequence for a gene product and is generally longer than the 5′ UTR. In certain embodiments, the 3′ UTR is about 200 nucleotides to about 800 nucleotides in length, but may optionally be longer or shorter.

As used herein, an “miRNA” refers to a microRNA which is a small non-coding RNA molecule which regulates mRNA and stops it from being translated to protein. Generally, hairpin-forming RNAs have a self-complementary “stem-loop” structure that includes a single nucleic acid encoding a stem portion having a duplex comprising a sense strand (e.g., passenger strand) connected to an antisense strand (e.g., guide strand) by a loop sequence. The passenger strand and the guide strand share complementarity. In some embodiments, the passenger strand and guide strand share 100% complementarity. In some embodiments, the passenger strand and guide strand share at least 50%, at least 60%, at least 70%, at least 80%, at least 90%, at least 95%, or at least 99% complementarity. A passenger strand and a guide strand may lack complementarity due to a base-pair mismatch. In some embodiments, the passenger strand and guide strand of a hairpin-forming RNA have at least 1, at least 2, at least 3, at least 4, at least 5, at least 6, at least 7 at least 8, at least 9, or at least 10 mismatches. Generally, the first 2-8 nucleotides of the stem (relative to the loop) are referred to as “seed” residues and play an important role in target recognition and binding. The first residue of the stem (relative to the loop) is referred to as the “anchor” residue. In some embodiments, hairpin-forming RNA have a mismatch at the anchor residue. As used herein, the miRNA contains a “seed sequence” which is a region of nucleotides which specifically binds to mRNA (e.g., in the endogenous hMfn2) by complementary base pairing, leading to destruction or silencing of the mRNA. Such silencing may result in downregulation rather than complete extinguishing of the endogenous hMfn2 Unless otherwise specified, the term “miRNA” encompasses artificial microRNA (amiRNA), which are artificially designed.

A “self-complementary nucleic acid” refers to a nucleic acid capable of hybridizing with itself (i.e., folding hack upon itself) to form a single-stranded duplex structure, due to the complementarity (e.g., base-pairing) of the nucleotides within the nucleic acid strand. Self-complementary nucleic acids can form a variety of secondary structures, such as hairpin loops, loops, bulges, junctions and internal bulges. Certain self-complementary nucleic acids (e.g., miRNA or AmiRNA) perform regulatory functions, such as gene silencing.

The encoded miRNA provided herein have been designed to specifically target the endogenous human mitofusin 2 gene in patients having CMT2A. In certain embodiments the miRNA coding sequence comprises an anti-sense sequence in the following table 1, SEQ ID NOs: 27-46, 68, and 89.

TABLE 1

| | Target | SEQ | miRNA | SEQ |
|---|---|---|---|---|
| miR # | hMfn2 Sequence | ID NO: | Antisense sequence | ID NO: |
| 538 | AGAACAGGTTCTGGACG | 47 | TTGACGTCCAG | 27 |
| | TCAA | | AACCTGTTCT | |
| 874 | CAACTCTAAGTGCCCAC | 48 | AGAAGTGGGCA | 28 |
| | TTCT | | CTTAGAGTTG | |
| 877 | CTCTAAGTGCCCACTTCT | 49 | TTCAGAAGTGG | 29 |
| | GAA | | GCACTTAGAG | |
| 946 | GCTGGACAGCTGGATTG | 50 | TTGTCAATCCA | 30 |
| | ACAA | | GCTGTCCAGC | |
| 1377 | CTGCAGTGAAGACCAAG | 51 | CAAACTTGGTC | 31 |
| | TTTG | | TTCACTGCAG | |
| 1686 | CTCCAGTAGTCCTCAAG | 52 | AAACCTTGAGG | 32 |
| | GTTT | | ACTACTGGAG | |
| 2115 | AGTTCATGGTTTCCATG | 53 | TAACCATGGAA | 33 |
| | GTTA | | ACCATGAACT | |
| 2164 | CTCCATGGGCATTCTTGT | 54 | ACAACAAGAAT | 34 |
| | TGT | | GCCCATGGAG | |
| 2390 | CAGGAACTGTCTGGGAC | 55 | AAAGGTCCCAG | 35 |
| | CTTT | | ACAGTTCCTG | |
| 2457 | AGGAAATTGCCGCCATG | 56 | TGTTCATGGCG | 36 |
| | AACA | | GCAATTTCCT | |
| 1693 | AGTCCTCAAGGTTTATA | 57 | TTCTTATAAACC | 68 |
| | AGAA | | TTGAGGACT | |
| | GTCAATCAATAGCCAAC | 58 | TGAGGTTGGCT | 37 |
| | CTCA | | ATTGATTGAC | |
| | AAGGTGTTGACTGTGTG | 59 | TTCTCACACAGT | 38 |
| | AGAA | | CAACACCTT | |
| | AGCAGGTTTACTGCGAG | 60 | TTTCCTCGCAGT | 39 |
| | GAAA | | AAACCTGCT | |
| | AAGACATTGAGTTCCAT | 61 | AGAAATGGAAC | 40 |
| | TTCT | | TCAATGTCTT | |
| | TCACAGGTGATGTCCTG | 62 | TGAACAGGACA | 41 |
| | TTCA | | TCACCTGTGA | |
| | GTTCACATACCTGCTTGT | 63 | AATACAAGCAG | 42 |
| | ATT | | GTATGTGAAC | |
| | GGCTCGGGAGCAGCAGG | 64 | TAAACCTGCTG | 43 |
| | TTTA | | CTCCCGAGCC | |
| | TGGGCTCTATGGCCTCC | 65 | TAGAGGAGGCC | 44 |
| | TCTA | | ATAGAGCCCA | |
| | CAATTGCTTCCTGCGGG | 66 | TCTACCCGCAG | 45 |
| | TAGA | | GAAGCAATTG | |
| | TTCTTTGTGTCTGCTAAG | 67 | CTCCTTAGCAG | 46 |
| | GAG | | ACACAAAGAA | |

In certain embodiments, the seed sequence is 100% identical to the antisense sequence describe in the table. In certain embodiments, the seed sequence is located on the mature miRNA (5′ to 3′) and is generally starts at position 2 to 7, 2 to 8, or about 6 nucleotides from the 5′ end of the miRNA sense strand (from the 5′ end of the sense (+) strand) of the miRNA, although it may be longer than in length. In certain embodiments, the length of the seed sequence is no less than about 30% of the length of the miRNA sequence, which may be at least 7 nucleotides to about 28 nucleotides in length, at least 8 nucleotides to about 28 nucleotides in length, 7 nucleotides to 28 nucleotides, 8 nucleotides to 18 nucleotides, 12 nucleotides to 28 nucleotides in length, about 20 to about 26 nucleotides, about 21 nucleotides, about 24 nucleotides, or about 26 nucleotides. In the examples provided herein, the miRNA is delivered in the form of a stem-loop miRNA precursor sequences, e.g., about 50 to about 80 nucleotides in length, or about 55 nucleotides to about 70 nucleotides, or 60 to 65 nucleotides in length. In certain embodiments, this miRNA precursor comprises about 5 nucleotides, about a 21-nucleotide seed sequence, about a 19 nucleotide stem loop and about a 19 nucleotide sense sequence, wherein the sense sequence corresponds to the anti-sense sequence with one or two nucleotides being mismatched. An example of a suitable miRNA coding sequence is the miR1693 sequence of SEQ ID NO: 15. In certain embodiments, the miRNA coding sequence comprises SEQ ID NO: 15 (miR1693, 64 nt); an miRNA coding sequence comprising at least 60 consecutive nucleotides of SEQ ID NO: 15; or an miRNA coding sequence comprising at least 99% identity to SEQ ID NO: 15 which comprises a sequence with 100% identity to about nucleotide 6 to about nucleotide 26 of SEQ ID NO: 15 (or SEQ ID NO: 68). In certain embodiments, another sequences of the table above may be substituted in positions 6 to 26 of SEQ ID NO: 15 (or SEQ ID NO: 68). In still another embodiment, positions 6 to 26 of SEQ ID NO: 15 are retained, and an alternative sequence is selected for the stem-loop sequence. In certain embodiments, the miRNA coding sequence comprises SEQ ID NO: 16 (miR1518, 59 nt), a sequence comprising at least 99% identity to SEQ ID NO: 16. In certain embodiments, the miRNA coding sequence comprises SEQ ID NO: 89 (miR538, 59 nt), or a sequence comprising at least 99% identity to SEQ ID NO: 89. In certain embodiments, alternative stem-loop sequences are selected, wherein an antisense strand of stem is nt 6 to 26 of SEQ ID NO: 16, or nt 1 to 21 of SEQ ID NO: 89, and wherein loop sequence is nt 27 to 45 of SEQ ID NO: 41 or nt 22 to 40 od SEQ ID NO: 89.

In certain embodiments, the nucleic acid molecules (e.g., an expression cassette or vector genome) may contain more than one miRNA coding sequence. Such may comprise an miRNA coding sequence having the sequence of one, two or more of: (a) an miRNA coding sequence comprising SEQ ID NO:15 (miR1693, 64 nt); (b) an miRNA coding sequence comprising at least 60 consecutive nucleotides of SEQ ID NO: 15; (c) an miRNA coding sequence comprising at least 99% identity to SEQ ID NO:15 which comprises a sequence with 100% identity to about nucleotide 6 to about nucleotide 26 of SEQ ID NO: 15 (or SEQ ID NO: 68); and/or (d) an miRNA coding sequence comprising one or more of:

(i) TTGACGTCCAGAACCTGTTCT, SEQ ID NO: 27; (ii) AGAAGTGGGCACTTAGAGTTG, SEQ ID NO: 28; (iii) TTCAGAAGTGGGCACTTAGAG, SEQ ID NO: 29; (iv) TTGTCAATCCAGCTGTCCAGC, SEQ ID NO: 30; (v) CAAACTTGGTCTTCACTGCAG, SEQ ID NO: 31; (vi) AAACCTTGAGGACTACTGGAG, SEQ ID NO: 32; (vii) TAACCATGGAAACCATGAACT, SEQ ID NO: 33; (viii) ACAACAAGAATGCCCATGGAG, SEQ ID NO: 34; (ix) AAAGGTCCCAGACAGTTCCTG, SEQ ID NO: 35; (x) TGTTCATGGCGGCAATTTCCT, SEQ ID NO: 36; (xi) TGAGGTTGGCTATTGATTGAC, SEQ ID NO: 37; (xii) TTCTCACACAGTCAACACCTT, SEQ ID NO: 38; (xiii) TTTCCTCGCAGTAAACCTGCT, SEQ ID NO: 39; (xiv) AGAAATGGAACTCAATGTCTT, SEQ ID NO: 40; (xv) TGAACAGGACATCACCTGTGA, SEQ ID NO: 41; (xvi) AATACAAGCAGGTATGTGAAC, SEQ ID NO: 42; (xvii) TAAACCTGCTGCTCCCGAGCC, SEQ ID NO: 43; (xviii) TAGAGGAGGCCATAGAGCCCA, SEQ ID NO: 44; (xix) TCTACCCGCAGGAAGCAATTG, SEQ ID NO: 45; or (xx) CTCCTTAGCAGACACAAAGAA, SEQ ID NO: 46, or combinations of any of (i) through (xx). In certain embodiments, the nucleic acid molecules (e.g., an expression cassette or vector genome) may contain one, two or more miRNA coding sequence of SEQ ID NO: 16 (miR1518). In certain embodiments, the nucleic acid molecules (e.g., an expression cassette or vector genome) may contain one, two or more miRNA coding sequence of SEQ ID NO: 89 (miR538).

As used herein, an “miRNA target sequence” is a sequence located on the DNA positive strand (5′ to 3′) (e.g., of hMfn2) and is at least partially complementary to a miRNA sequence, including the miRNA seed sequence. The miRNA target sequence is exogenous to the untranslated region of the encoded transgene product and is designed to be specifically targeted by miRNA in cells in which repression of transgene expression is desired. Without wishing to be bound by theory, because hMfn2 is a ubiquitous protein and excess expression may be associated with toxicity and/or other negative side effects, the miRNA preferentially target the endogenous hMfn2 gene while avoiding targeting the engineered hMfn2 gene which is delivered to the CMT2A patent. More particularly, the sequences encoding the hMfn2 which are delivered via a vector are designed to contain altered codon sequences at the target site.

Typically, the miRNA target sequence is at least 7 nucleotides to about 28 nucleotides in length, at least 8 nucleotides to about 28 nucleotides in length, 7 nucleotides to 28 nucleotides, 8 nucleotides to 18 nucleotides, 12 nucleotides to 28 nucleotides in length, about to about 26 nucleotides, about 22 nucleotides, about 24 nucleotides, or about 26 nucleotides, and which contains at least one consecutive region (e.g., 7 or 8 nucleotides) which is complementary to the miRNA seed sequence. In certain embodiments, the target sequence comprises a sequence with exact complementarity (100%) or partial complementarity to the miRNA seed sequence with some mismatches. In certain embodiments, the target sequence comprises at least 7 to 8 nucleotides which are 100% complementary to the miRNA seed sequence. In certain embodiments, the target sequence consists of a sequence which is 100% complementary to the miRNA seed sequence. In certain embodiments, the target sequence contains multiple copies (e.g., two or three copies) of the sequence which is 100% complementary to the seed sequence. In certain embodiments, the region of 100% complementarity comprises at least 30% of the length of the target sequence. In certain embodiments, the remainder of the target sequence has at least about 80% to about 99% complementarity to the miRNA. In certain embodiments, in an expression cassette containing a DNA positive strand, the miRNA target sequence is the reverse complement of the miRNA.

Thus, the sequences provided herein which are 95% to 99.9% identical to the Mfn2 coding sequences of SEQ ID NO: 11 and 24, are designed to avoid reverting to a native human sequence to which a selected miRNA in the construct is targeted.

In certain embodiments, the miRNA preferentially targets the endogenous hMfn2 gene while avoiding targeting the engineered hMfn2 gene, wherein the endogenous hMfn2 nucleic acid sequence is of SEQ ID NO: 18. In certain embodiments, the miRNA coding sequence comprises one or more of: (i) TTGACGTCCAGAACCTGTTCT, SEQ ID NO: 27, targeting nt 216-236 of SEQ ID NO: 18; (ii) AGAAGTGGGCACTTAGAGTTG, SEQ ID NO: 28, targeting nt 552-572 of SEQ ID No: 18; (iii) TTCAGAAGTGGGCACTTAGAG, SEQ ID NO: 29, targeting nt 555-575 of SEQ ID NO: 18; (iv) TTGTCAATCCAGCTGTCCAGC, SEQ ID NO: 30, targeting nt 624-644 of SEQ ID NO: 18; (v) CAAACTTGGTCTTCACTGCAG, SEQ ID NO: 31, targeting nt 1055-1075 of SEQ ID NO: 18; (vi) AAACCTTGAGGACTACTGGAG, SEQ ID NO: 32, targeting nt 1364-1384 of SEQ ID NO: 18; (vii) TAACCATGGAAACCATGAACT, SEQ ID NO: 33, targeting nt 1793-1813 of SEQ ID NO: 18; (viii) ACAACAAGAATGCCCATGGAG, SEQ ID NO: 34, targeting nt 1842-1862 of SEQ ID NO: 18; (ix) AAAGGTCCCAGACAGTTCCTG, SEQ ID NO: 35, targeting nt 2068-2088 of SEQ ID NO: 181; (x) TGTTCATGGCGGCAATTTCCT, SEQ ID NO: 36, targeting nt 2135-2155 of SEQ ID NO: 18; (xi) TGAGGTTGGCTATTGATTGAC, SEQ ID NO: 37, targeting 5′UTR; (xii) TTCTCACACAGTCAACACCTT, SEQ ID NO: 38, targeting 3′UTR; (xiii) TTTCCTCGCAGTAAACCTGCT, SEQ ID NO: 39, targeting nt 1157-1177 of SEQ ID NO: 18; (xiv) AGAAATGGAACTCAATGTCTT, SEQ ID NO: 40, targeting nt 1616-1636 of SEQ ID NO: 18; (xv) TGAACAGGACATCACCTGTGA, SEQ ID NO: 41, targeting 3′UTR; (xvi) AATACAAGCAGGTATGTGAAC, SEQ ID NO: 42, targeting 3′UTR; (xvii) TAAACCTGCTGCTCCCGAGCC, SEQ ID NO: 43, targeting nt 1146-1166 of SEQ ID NO: 18; (xviii) TAGAGGAGGCCATAGAGCCCA, SEQ ID NO: 44, targeting nt 1914-1934 of SEQ ID NO: 18; (xix) TCTACCCGCAGGAAGCAATTG, SEQ ID NO: 45, targeting nt 390-410 of SEQ ID NO: 18; or (xx) CTCCTTAGCAGACACAAAGAA, SEQ ID NO: 46, targeting nt 904-924 of SEQ ID NO: 18.

In certain embodiments, the engineered hMfn2 nucleic acid sequence is of SEQ ID NO: 11 or 24. In certain embodiments the engineered hMfn2 nucleic acid sequence is of SEQ ID NO: 18 wherein 1, 2, 3, or 4 nucleotide mismatches are present in the regions of nucleotides: (i) nt 216-236 of SEQ ID NO: 18; (ii) nt 552-572 of SEQ ID NO: 18; (iii) nt 555-575 of SEQ ID NO: 18; (iv) nt 624-644 of SEQ ID NO: 18; (v) nt 1055-1075 of SEQ ID NO: 18; (vi) nt 1364-1384 of SEQ ID NO: 18; (vii) nt 1793-1813 of SEQ ID NO: 18; (viii) nt 1842-1862 of SEQ ID NO: 18; (ix) nt 2068-2088 of SEQ ID NO: 18; (x) nt 2135-2155 of SEQ ID NO: 18; (xi) nt 1157-1177 of SEQ ID NO: 18; (xii) nt 1616-1636 of SEQ ID NO: 18; (xiii) nt 1146-1166 of SEQ ID NO: 18; (xiv) nt 1914-1934 of SEQ ID NO: 18; (xv) nt 390-410 of SEQ ID NO: 18; or (xvi) nt 904-924 of SEQ ID NO: 18.

In certain embodiments, a single nucleic acid (e.g., an expression cassette or vector genome containing same) contains both the engineered hMfn2 coding sequence and at least one miRNA coding sequence, wherein the miRNA is specifically targeted to a region of the endogenous human Mfn2 sequence not present in the engineered hMfn2 sequence. In certain embodiments, the human mitofusin 2 coding sequence is upstream (5′) of the at least one miRNA and these two elements are separated by a spacer or linker sequence. In certain embodiments, there is at least 75 nucleotides between the stop codon of the hMfn2 coding sequence and the start of the most 5′ miRNA coding sequence. In certain embodiments, the spacer is about 75 nucleotides to about 300 nucleotides, or about 75 nucleotides to about 250 nucleotides, or about 75 nucleotides to about 200 nucleotides, or about 75 nucleotides to about 150 nucleotides, or about 75 nucleotides to about 100 nucleotides, or about 80 nucleotides to about 300 nucleotides, or about 80 nucleotides to about 250 nucleotides, or about 80 nucleotides to about 200 nucleotides, or about 80 nucleotides to about 150 nucleotides, or about 80 nucleotides to about 100 nucleotides. Optionally, the engineered hMfn2 coding sequence and the at least one miRNA coding sequence are separated by about 75 nucleotides. Suitably, the spacer sequence is a non-coding sequence which lacks any restriction enzyme sites. Optionally, the spacer may include one or more intron sequences. In certain embodiments, one or more of the miRNA sequences may be located within the intron. In certain embodiments, the linker sequence is SEQ ID NO: 17. In certain embodiments, the linker sequence is SEQ ID NO: 90.

In certain embodiments, the engineered hMfn2 coding sequence and the miRNA coding sequence(s) are delivered via different nucleic acid sequences, e.g., two or more different vectors, a combination comprising a vector and an LNP, etc. In certain embodiments, the two different vectors are AAV vectors. In certain embodiments, these vectors have different expression cassettes. In other embodiments, these vectors have the same capsid. In other embodiments, the vectors have different embodiments. In certain embodiments, the miRNA coding sequence(s) are delivered via an LNP or another non-viral delivery system. In certain embodiments, the engineered hMfn2 sequence is delivered via an LNP or another non-viral delivery system. In certain embodiments, combinations of two or more different delivery systems (e.g., viral and non-viral, two different non-viral) are used. In these and other embodiments, the two or more different vectors or other delivery systems may be administered substantially simultaneously, or one or more of these systems may be delivered before the other. In certain embodiments, the engineered hMfn2 sequence is SEQ ID NO: 11, or a sequence 90% to 100% identical thereto which encodes an mRNA which is not bound by the miR with which it is co-administered and which encodes hMfn2. In certain embodiments, the engineered hMfn2 sequence is SEQ ID NO: 24, or a sequence 90% to 100% identical thereto which encodes an mRNA which is not bound by the miR with which it is co-administered and which encodes hMfn2. In certain embodiments, the miR is miR538 having the sequence of SEQ ID NO: 89 which targets endogenous hMfn2 in the subject, but which does not target the engineered hMfn2 cDNA sequence or engineered encoded mRNA sequence.

As used herein, the terms “AAV.hMfn2” or “rAAV.hMfn2” are used to refer to a recombinant adeno-associated virus which has an AAV capsid having therewithin a vector genome comprising a human mitofusin 2 coding sequence (e.g., a cDNA) under the control of regulatory sequences. As used herein, the terms “AAV.hMfn2 miRXXX” or “rAAV.hMfn2 miRXXX” are used to refer to a recombinant adeno-associated virus which has an AAV capsid having therewithin a vector genome comprising an miR targeting an endogenous human mitofusin 2 coding sequence.

Specific capsid types may be specified, such as, e.g., AAV1.hMfn2 or rAAV1.hMfn2, which refers to a recombinant AAV having an AAV1 capsid; AAVhu68.hMfn2 or AAVhu68.Mfn2, which refers to recombinant AAV having an AAVhu68 capsid. AAVrh91.hMfn2 or AAVrh91.Mfn2, which refers to recombinant AAV having an AAVrh91 capsid.

A “recombinant AAV” or “rAAV” is a DNAse-resistant viral particle containing two elements, an AAV capsid and a vector genome containing at least non-AAV coding sequences packaged within the AAV capsid. Unless otherwise specified, this term may be used interchangeably with the phrase “rAAV vector”. The rAAV is a “replication-defective virus” or “viral vector”, as it lacks any functional AAV rep gene or functional AAV cap gene and cannot generate progeny. In certain embodiments, the only AAV sequences are the AAV inverted terminal repeat sequences (ITRs), typically located at the extreme 5′ and 3′ ends of the vector genome in order to allow the gene and regulatory sequences located between the ITRs to be packaged within the AAV capsid. Generally, an AAV capsid is composed of 60 capsid (cap) protein subunits, VP1, VP2, and VP3, that are arranged in an icosahedral symmetry in a ratio of approximately 1:1:10 to 1:1:20, depending upon the selected AAV. Various AAVs may be selected as sources for capsids of AAV viral vectors as identified above. In one embodiment, the AAV capsid is an AAV9 capsid or an engineered variant thereof. In certain embodiments, the variant AAV9 capsid is an AAV9.PhP.eB capsid (nucleic acid sequence of SEQ ID NO: 84; amino acid sequence of SEQ ID NO: 85). In certain embodiments, the PhP.eB capsid is selected for use in mouse studies and is a suitable model for a Glade F vector (e.g., AAVhu68) in humans. In certain embodiments, the capsid protein is designated by a number or a combination of numbers and letters following the term “AAV” in the name of the rAAV vector. Unless otherwise specified, the AAV capsid, ITRs, and other selected AAV components described herein, may be readily selected from among any AAV, including, without limitation, the AAVs identified as AAV1, AAV2, AAV3, AAV4, AAV5, AAV6, AAV7, AAV8, AAV9, AAVrh10, AAVhu37, AAVrh32.33, AAV8 bp, AAV7M8 and AAVAnc80, AAV1, AAV2, AAV3, AAV4, AAV5, AAV6, AAV7, AAV8, AAV9.47, AAV9(hu14), AAV10, AAV11, AAV12, AAVrh8, AAVrh74, AAV-DJ8, AAV-DJ, AAVhu68, and AAV9 variants (e.g., U.S. Provisional Application No. 63/119,863, filed Dec. 1, 2020), without limitation. See, e.g., WO 2019/168961 and WO 2019/169004, both for Novel AAV Vectors Having Reduced Capsid Deamidation and Uses Therefor; US Published Patent Application No. 2007-0036760-A1; US Published Patent Application No. 2009-0197338-A1; EP 1310571. See also, WO 2003/042397 (AAV7 and other simian AAV), U.S. Pat. Nos. 7,790,449 and 7,282,199 (AAV8), WO 2005/033321 and U.S. Pat. No. 7,906,111 (AAV9), and WO 2006/110689, and WO 2003/042397 (rh.10), WO 2005/033321, WO 2018/160582 (AAVhu68), which are incorporated herein by reference. See, also WO 2019/168961 and WO 2019/169004, describing deamidation profiles for these and other AAV capsids. Other suitable AAVs may include, without limitation, AAVrh90 [PCT/US20/30273, filed Apr. 28, 2020], AAVrh91 [PCT/US20/30266, filed Apr. 28, 2020 and U.S. Provisional Patent Applications No. 63/109,734, filed Nov. 4, 2020 and US Provisional Patent Application No. 63/065,616, filed Aug. 14, 2020] AAVrh92, AAVrh93, AAVrh91.93 [PCT/US20/30281, filed Apr. 28, 2020], which are incorporated by reference herein. Other suitable AAV include AAV3B variants which are described in PCT/US20/56511, filed Oct. 20, 2020, which claims the benefit of U.S. Provisional Patent Application No. 62/924,112, filed Oct. 21, 2019, and U.S. Provisional Patent Application No. 63/025,753, filed May 15, 2020, describing AAV3B.AR2.01, AAV3B.AR2.02, AAV3B.AR2.03, AAV3B.AR2.04, AAV3B.AR2.05, AAV3B.AR2.06, AAV3B.AR2.07, AAV3B.AR2.08, AAV3B.AR2.10, AAV3B.AR2.11, AAV3B.AR2.12, AAV3B.AR2.13, AAV3B.AR2.14, AAV3B.AR2.15, AAV3B.AR2.16, or AAV3B.AR2.17, which are incorporated herein by reference. These documents also describe other AAV capsids which may be selected for generating rAAV and are incorporated by reference. Among the AAVs isolated or engineered from human or non-human primates (NHP) and well characterized, human AAV2 is the first AAV that was developed as a gene transfer vector; it has been widely used for efficient gene transfer experiments in different target tissues and animal models.

As used herein, a “vector genome” refers to the nucleic acid sequence packaged inside a parvovirus (e.g., rAAV) capsid which forms a viral particle. Such a nucleic acid sequence contains AAV inverted terminal repeat sequences (ITRs). In the examples herein, a vector genome contains, at a minimum, from 5′ to 3′, an AAV 5′ ITR, coding sequence(s) (i.e., transgene(s)), and an AAV 3′ ITR. ITRs from AAV2, a different source AAV than the capsid, or other than full-length ITRs may be selected. In certain embodiments, the ITRs are from the same AAV source as the AAV which provides the rep function during production or a transcomplementing AAV. Further, other ITRs, e.g., self-complementary (scAAV) ITRs, may be used. Both single-stranded AAV and self-complementary (sc) AAV are encompassed with the rAAV. The transgene is a nucleic acid coding sequence, heterologous to the vector sequences, which encodes a polypeptide, protein, functional RNA molecule (e.g., miRNA, miRNA inhibitor) or other gene product, of interest. The nucleic acid coding sequence is operatively linked to regulatory components in a manner which permits transgene transcription, translation, and/or expression in a cell of a target tissue. Suitable components of a vector genome are discussed in more detail herein.

In one example, a “vector genome” contains, at a minimum, from 5′ to 3′, a vector-specific sequence, a nucleic acid sequence comprising an engineered human Mfn2 coding sequence and optionally an miRNA sequences targeting the endogenous Mfn2 operably linked to regulatory control sequences (which direct their expression in a target cell), where the vector-specific sequence may be a terminal repeat sequence which specifically packages the vector genome into a viral vector capsid or envelope protein. For example, AAV inverted terminal repeats are utilized for packaging into AAV and certain other parvovirus capsids.

In certain embodiments, a composition is provided which comprises an aqueous liquid suitable for intrathecal injection and a stock of vector (e.g., rAAV having a AAV capsid which preferentially targets cells in the central nervous system and/or the dorsal root ganglia (e.g., CNS, including, e.g., nerve cells (such as, pyramidal, purkinje, granule, spindle, and interneuron cells) and glia cells (such as astrocytes, oligodendrocytes, microglia, and ependymal cells), wherein the vector having an engineered hMfn2 coding sequence and/or an at least one miRNA specific endogenous hMfn2 for delivery to the central nervous system (CNS). In certain embodiments, the composition comprising one or more vectors as described herein is formulated for sub-occipital injection into the cisterna magna (intra-cisterna magna). In certain embodiments, the composition is administered via a computed tomography- (CT-) rAAV injection. In certain embodiments, the composition is administered using Ommaya reservoir. In certain embodiments, the patient is administered a single dose of the composition.

As used herein, an “expression cassette” refers to a nucleic acid molecule which comprises a biologically useful nucleic acid sequence (e.g., a gene cDNA encoding a protein, enzyme or other useful gene product, mRNA, etc.) and regulatory sequences operably linked thereto which direct or modulate transcription, translation, and/or expression of the nucleic acid sequence and its gene product. As used herein, “operably linked” sequences include both regulatory sequences that are contiguous or non-contiguous with the nucleic acid sequence and regulatory sequences that act in trans or cis nucleic acid sequence. Such regulatory sequences typically include, e.g., one or more of a promoter, an enhancer, an intron, a Kozak sequence, a polyadenylation sequence, and a TATA signal. The expression cassette may contain regulatory sequences upstream (5′ to) of the gene sequence, e.g., one or more of a promoter, an enhancer, an intron, etc., and one or more of an enhancer, or regulatory sequences downstream (3′ to) a gene sequence, e.g., 3′ untranslated region (3′ UTR) comprising a polyadenylation site, among other elements. In certain embodiments, the regulatory sequences are operably linked to the nucleic acid sequence of a gene product, wherein the regulatory sequences are separated from nucleic acid sequence of a gene product by an intervening nucleic acid sequences, i.e., 5′-untranslated regions (5′UTR). In certain embodiments, the expression cassette comprises nucleic acid sequence of one or more of gene products. In some embodiments, the expression cassette can be a monocistronic or a bicistronic expression cassette. In other embodiments, the term “transgene” refers to one or more DNA sequences from an exogenous source which are inserted into a target cell.

Typically, such an expression cassette can be used for generating a viral vector and contains the coding sequence for the gene product described herein flanked by packaging signals of the viral genome and other expression control sequences such as those described herein. In certain embodiments, a vector genome may contain two or more expression cassettes.

In certain embodiment expression cassette comprises the hMfn2 coding sequences (and/or miRNA sequences targeting the endogenous Mfn2), promoter, and may include other regulatory sequences therefor, which cassette may be packaged into a vector (e.g., rAAV, lentivirus, retrovirus, etc.).

### AAV

Recombinant parvoviruses are particularly well suited as vectors for treatment of CMT2A. As described herein, recombinant parvoviruses may contain an AAV capsid (or bocavirus capsid). In certain embodiments, the capsid targets cells within the dorsal root ganglion and/or cells within the lower motor neurons and/or primary sensory neurons. In certain embodiments, compositions provided herein may have a single rAAV stock which comprises an rAAV comprising an engineered hMfn2 and an miRNA specifically targeting endogenous hMfn2 in order to downregulate the endogenous hMfn2 levels and to reduce any toxicity associated with overexpression of hMfn2. In other embodiments, an rAAV may be comprise the hMfn2 and may be co-administered with a different vector comprising an miRNA which downregulates endogenous hMfn2 In other embodiments, an rAAV may be comprise the at least one miRNA which downregulates endogenous hMfn2 and a second vector (or other composition) delivers the hMfn2.

For example, vectors generated using AAV capsids from Clade F (e.g., AAVhu68 or AAV9) can be used to produce vectors which target and express hMfn2 in the CNS. Alternatively, vectors generated using AAV capsids from Clade A (e.g., AAV1, AAVrh91) may be selected. In still other embodiments, other parvovirus or other AAV viruses may be suitable sources of AAV capsids.

An AAV1 capsid refers to a capsid having AAV vp1 proteins, AAV vp2 proteins and AAV vp3 proteins. In particular embodiments, the AAV1 capsid comprises a pre-determined ratio of AAV vp1 proteins, AAV vp2 proteins and AAV vp3 proteins of about 1:1:10 assembled into a T1 icosahedron capsid of 60 total vp proteins. An AAV1 capsid is capable of packaging genomic sequences to form an AAV particle (e.g., a recombinant AAV where the genome is a vector genome). Typically, the capsid nucleic acid sequences encoding the longest of the vp proteins, i.e., VP1, is expressed in trans during production of an rAAV having an AAV1 capsid are described in, e.g., U.S. Pat. Nos. 6,759,237, 7,105,345, 7,186,552, 8,637,255, and 9,567,607, which are incorporated herein by reference. See, also, WO 2018/168961, which is incorporated by reference. In certain embodiments, AAV1 is characterized by a capsid composition of a heterogenous population of VP isoforms which are deamidated as defined in WO 2018/160582, incorporated herein by reference in its entirety, based on the total amount of VP proteins in the capsid, as determined using mass spectrometry. In certain embodiments, the AAV capsid is modified at one or more of the following positions, in the ranges provided below, as determined using mass spectrometry. Suitable modifications include those described in the paragraph above labelled modulation of deamidation, which is incorporated herein. In certain embodiments, one or more of the following positions, or the glycine following the N is modified as described herein. In certain embodiments, an AAV1 mutant is constructed in which the glycine following the N at position 57, 383, 512 and/or 718 are preserved (i.e., remain unmodified). In certain embodiments, the NG at the four positions identified in the preceding sentence are preserved with the native sequence. In certain embodiments, an artificial NG is introduced into a different position than one of the positions as defined and identified in WO 2018/160582, incorporated herein by reference.

As used herein, an AAVhu68 capsid refers to a capsid as defined in WO 2018/160582, incorporated herein by reference. As described herein, a rAAVhu68 has a rAAVhu68 capsid produced in a production system expressing capsids from an AAVhu68 nucleic acid. In certain embodiments, the AAVhu68 nucleic acid sequence is SEQ ID NO: 81, encoding and for an amino acid sequence of SEQ ID NO 82. In certain embodiments, the AAVhu68 nucleic acid sequence is SEQ ID NO: 83, encoding for an amino acid sequence of SEQ ID NO: 82. The rAAVhu68 resulting from production using a single nucleic acid sequence vp1 produces the heterogenous populations of vp1 proteins, vp2 proteins and vp3 proteins. These subpopulations include, at a minimum, deamidated asparagine (N or Asn) residues. For example, asparagines in asparagine-glycine pairs are highly deamidated. In certain embodiments, the vp2 and/or vp3 proteins may be expressed additionally or alternatively from different nucleic acid sequences than the vp1, e.g., to alter the ratio of the vp proteins in a selected expression system.

Genomic sequences which are packaged into an AAV capsid and delivered to a host cell are typically composed of, at a minimum, a transgene and its regulatory sequences, and AAV inverted terminal repeats (ITRs). Both single-stranded AAV and self-complementary (sc) AAV are encompassed with the rAAV. The transgene is a nucleic acid coding sequence, heterologous to the vector sequences, which encodes a polypeptide, protein, functional RNA molecule (e.g., miRNA, miRNA inhibitor) or other gene product, of interest. The nucleic acid coding sequence is operatively linked to regulatory components in a manner which permits transgene transcription, translation, and/or expression in a cell of a target tissue.

The AAV sequences of the vector typically comprise the cis-acting 5′ and 3′ inverted terminal repeat sequences (See, e.g., B. J. Carter, in “Handbook of Parvoviruses”, ed., P. Tijsser, CRC Press, pp. 155 168 (1990)). The ITR sequences are about 145 bp in length.

Preferably, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J. Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences.

The ITRs are the genetic elements responsible for the replication and packaging of the genome during vector production and are the only viral cis elements required to generate rAAV. In one embodiment, the ITRs are from an AAV different than that supplying a capsid. In a preferred embodiment, the ITR sequences from AAV2, or the deleted version thereof (ΔITR), which may be used for convenience and to accelerate regulatory approval. However, ITRs from other AAV sources may be selected. Where the source of the ITRs is from AAV2 and the AAV capsid is from another AAV source, the resulting vector may be termed pseudotyped. Typically, AAV vector genome comprises an AAV 5′ ITR, the nucleic acid sequences encoding the gene product(s) and any regulatory sequences, and an AAV 3′ ITR. However, other configurations of these elements may be suitable. In one embodiment, a self-complementary AAV is provided. A shortened version of the 5′ ITR, termed AITR, has been described in which the D-sequence and terminal resolution site (trs) are deleted. In certain embodiments, the vector genome includes a shortened AAV2 ITR of 130 base pairs, wherein the external “a” element is deleted. The shortened ITR is reverted back to the wild-type length of 145 base pairs during vector DNA amplification using the internal A element as a template. In other embodiments, the full-length AAV 5′ and 3′ ITRs are used.

In addition to the major elements identified above for the vector (e.g., an rAAV), the vector also includes conventional control elements necessary which are operably linked to the transgene in a manner which permits its transcription, translation and/or expression in a cell. As used herein, the term “expression” or “gene expression” refers to the process by which information from a gene is used in the synthesis of a functional gene product. The gene product may be a protein, a peptide, or a nucleic acid polymer (such as an RNA, a DNA or a PNA).

As used herein, the term “regulatory sequence”, or “expression control sequence” refers to nucleic acid sequences, such as initiator sequences, enhancer sequences, and promoter sequences, which induce, repress, or otherwise control the transcription of protein encoding nucleic acid sequences to which they are operably linked. The regulatory control elements typically contain a promoter sequence as part of the expression control sequences, e.g., located between the selected 5′ ITR sequence and the coding sequence. In particularly desirable embodiments, a tissues specific promoter for the central nervous system is selected. For example, the promoter may be a neural cell promoter, e.g., gfaABC(1)D promoter (Addgene #50473)), or the human Syn promoter (the sequence is available from Addgene, Ref #50465).

Other suitable promoters may include, e.g., constitutive promoters, regulatable promoters [see, e.g., WO 2011/126808 and WO 2013/04943], tissue specific promoters, or a promoter responsive to physiologic cues may be used may be utilized in the vectors described herein. The promoter(s) can be selected from different sources, e.g., human cytomegalovirus (CMV) immediate-early enhancer/promoter, the SV40 early enhancer/promoter, the JC polymovirus promoter, myelin basic protein (MBP) or glial fibrillary acidic protein (GFAP) promoters, herpes simplex virus (HSV-1) latency associated promoter (LAP), rouse sarcoma virus (RSV) long terminal repeat (LTR) promoter, neuron-specific promoter (NSE), platelet derived growth factor (PDGF) promoter, hSYN, melanin-concentrating hormone (MCH) promoter, CBA, matrix metalloprotein promoter (MPP), and the chicken beta-actin promoter. In addition to a promoter a vector may contain one or more other appropriate transcription initiation, termination, enhancer sequences, efficient RNA processing signals such as splicing and polyadenylation (polyA) signals; sequences that stabilize cytoplasmic mRNA for example WPRE; sequences that enhance translation efficiency (i.e., Kozak consensus sequence); sequences that enhance protein stability; and when desired, sequences that enhance secretion of the encoded product. An example of a suitable enhancer is the CMV enhancer. Other suitable enhancers include those that are appropriate for desired target tissue indications. In one embodiment, the expression cassette comprises one or more expression enhancers. In one embodiment, the expression cassette contains two or more expression enhancers. These enhancers may be the same or may differ from one another. For example, an enhancer may include a CMV immediate early enhancer. This enhancer may be present in two copies which are located adjacent to one another. Alternatively, the dual copies of the enhancer may be separated by one or more sequences. In still another embodiment, the expression cassette further contains an intron, e.g., the chicken beta-actin intron. Other suitable introns include those known in the art, e.g., such as are described in WO 2011/126808. Examples of suitable polyA sequences include, e.g., SV40, SV50, bovine growth hormone (bGH), human growth hormone, and synthetic polyAs. In certain embodiments, the polyA is SV40 polyA. In certain embodiments, the polyA is rabbit globin poly A (RBG). Optionally, one or more sequences may be selected to stabilize mRNA. An example of such a sequence is a modified WPRE sequence, which may be engineered upstream of the polyA sequence and downstream of the coding sequence [see, e.g., MA Zanta-Boussif, et al, Gene Therapy (2009) 16: 605-619.

In certain embodiments, the vector genome comprises a tissue specific promoter In some embodiments, the tissue specific promoter is a human synapsin promoter. In certain embodiments, the human synapsin promoter comprises nucleic acid sequence of SEQ ID NO: 6. In certain embodiments, the vector genome comprises a constitutive promoter, wherein the promoter is a CB7 promoter or a CAG promoter. In certain embodiments, the CB7 promoter comprises nucleic acid sequence of SEQ ID NO: 86. In certain embodiments, the CAG promoter comprises nucleic acid sequence of SEQ ID NO: 87.

In one embodiment, the vector genome comprises: an AAV 5′ ITR, a promoter, an optional enhancer, an optional intron, a coding sequence for human Mfn2 (hMfn2 or huMfn2) comprising same, a poly A, and an AAV 3′ ITR. In certain embodiments, the vector genome comprises: a AAV 5′ ITR, a promoter, an optional enhancer, an optional intron, a coding sequence for human Mfn2 comprising same, a poly A, and an AAV 3′ ITR. In certain embodiments, the vector genome comprises: a AAV 5′ ITR, a promoter, an optional enhancer, an optional intron, a huMfn2 coding sequence, a poly A, and an AAV 3′ ITR. In certain embodiments, the vector genome comprises: an AAV2 5′ ITR, an EF1a promoter, an optional enhancer, an optional promoter, huMfn2, an SV40 poly A, and an AAV2 3′ ITR. In certain embodiments, the vector genome is AAV2 5′ ITR, UbC promoter, optional enhancer, optional intron, huMfn2, an SV40 poly A, and an AAV2 3′ ITR. In certain embodiments, the vector genome is AAV2 5′ ITR, CB7 promoter, an intron, huMfn2, an SV40 poly A, and an AAV2 3′ ITR. In certain embodiment, the vector genome is an AAV2 5′ ITR, CB7 promoter, intron, huMfn2, a rabbit beta globin poly A, and an AAV2 3′ ITR. In certain embodiment, the vector genome is an AAV2 5′ ITR, CB7 promoter, intron, an engineered huMfn2, a linker, a miR targeted to endogenous huMfn2 sequence, a rabbit beta globin poly A, and an AAV2 3′ ITR. In certain embodiment, the vector genome is an AAV2 5′ ITR, CB7 promoter, intron, an engineered huMfn2, a linker, a miR1518 sequence, a rabbit beta globin poly A, and an AAV2 3′ ITR. In certain embodiment, the vector genome is an AAV2 5′ ITR, CB7 promoter, intron, an engineered huMfn2, a linker, a miR538, a rabbit beta globin poly A, and an AAV2 3′ ITR. See, e.g., SEQ ID NOs: 1, 3, 69, 71, 73, 75, 77, and 79. The huMfn2 coding sequences are selected from those defined in the present specification. See, e.g., SEQ ID NO: 11 or a sequence 95% to 99.9% identical thereto, or SEQ ID NO: 11 or a sequence 95% to 99.9% identical thereto, or a fragment thereof as defined herein. Other elements of the vector genome or variations on these sequences may be selected for the vector genomes for certain embodiments of this invention.

### Vector Production

For use in producing an AAV viral vector (e.g., a recombinant (r) AAV), the expression cassettes can be carried on any suitable vector, e.g., a plasmid, which is delivered to a packaging host cell. The plasmids useful in this invention may be engineered such that they are suitable for replication and packaging in vitro in prokaryotic cells, insect cells, mammalian cells, among others. Suitable transfection techniques and packaging host cells are known and/or can be readily designed by one of skill in the art.

TABLE 2

| | SEQ ID NOs |
|---|---|
| AAV Vector | (vector genome) |
| Syn.PI.hMfn2eng.link.hMfn2.miR1693.WPRE.bGH | 1 |
| Syn.PI.rMnf2eng.link.rMfn2 miR1518 WPRE.BGH | 3 |
| CB7.CI.hMfn2.GA.RBG | 79 |
| CB7.CI.hMfn2.GA.LINK.miR1518.RBG | 77 |
| CB7.CI.hMfn2.GA.LINK.miR538.RBG | 75 |
| CAG.CI.hMfn2.GA.WPRE.SV40 | 73 |
| CAG.CI.hMfn2.GA.LINK.miR1518.WPRE.SV40 | 71 |
| CAG.CI.hMfn2.GA.LINK.miR538.WPRE.SV40 | 69 |

In certain embodiments, the production plasmid comprises a vector genome for packaging into a capsid which comprises: (a) an engineered nucleic acid sequence encoding human mitofusin 2; (b) a spacer sequence located between (a) and (c); (c) at least one miRNA sequence specific for endogenous human mitofusin 2 in a CMT2 patient located 3′ to the sequence of (a) and (b); wherein the engineered nucleic acid sequence of (a) lacks the target site for the at least one miRNA, thereby preventing the miRNA from targeting the engineered human mitofusin 2 coding sequence; (c) regulatory sequences operably linked to (a) and (c). In certain embodiments, the production plasmid comprises a vector genome comprising nucleic acid sequence of SEQ ID NO: 1, 3, 69, 71, 73, 75, 77, or 79.

Methods for generating and isolating AAVs suitable for use as vectors are known in the art. See generally, e.g., Grieger & Samulski, 2005, “Adeno-associated virus as a gene therapy vector: Vector development, production and clinical applications,” Adv. Biochem. Engin/Biotechnol. 99: 119-145; Buning et al., 2008, “Recent developments in adeno-associated virus vector technology,” J. Gene Med. 10:717-733; and the references cited below, each of which is incorporated herein by reference in its entirety. For packaging a transgene into virions, the ITRs are the only AAV components required in cis in the same construct as the nucleic acid molecule containing the expression cassettes. The cap and rep genes can be supplied in trans.

In one embodiment, the expression cassettes described herein are engineered into a genetic element (e.g., a shuttle plasmid) which transfers the immunoglobulin construct sequences carried thereon into a packaging host cell for production a viral vector. In one embodiment, the selected genetic element may be delivered to an AAV packaging cell by any suitable method, including transfection, electroporation, liposome delivery, membrane fusion techniques, high velocity DNA-coated pellets, viral infection and protoplast fusion. Stable AAV packaging cells can also be made. Alternatively, the expression cassettes may be used to generate a viral vector other than AAV, or for production of mixtures of antibodies in vitro. The methods used to make such constructs are known to those with skill in nucleic acid manipulation and include genetic engineering, recombinant engineering, and synthetic techniques. See, e.g., Molecular Cloning: A Laboratory Manual, ed. Green and Sambrook, Cold Spring Harbor Press, Cold Spring Harbor, N.Y. (2012).

The term “AAV intermediate” or “AAV vector intermediate” refers to an assembled rAAV capsid which lacks the desired genomic sequences packaged therein. These may also be termed an “empty” capsid. Such a capsid may contain no detectable genomic sequences of an expression cassette, or only partially packaged genomic sequences which are insufficient to achieve expression of the gene product. These empty capsids are non-functional to transfer the gene of interest to a host cell.

The recombinant adeno-associated virus (AAV) described herein may be generated using techniques which are known. See, e.g., WO 2003/042397; WO 2005/033321, WO 2006/110689; U.S. Pat. No. 7,588,772 B2. Such a method involves culturing a host cell which contains a nucleic acid sequence encoding an AAV capsid protein; a functional rep gene; an expression cassette as described herein flanked by AAV inverted terminal repeats (ITRs) and a transgene; and sufficient helper functions to permit packaging of the expression cassette into the AAV capsid protein. Also provided herein is the host cell which contains a nucleic acid sequence encoding an AAV capsid; a functional rep gene; a vector genome as described; and sufficient helper functions to permit packaging of the vector genome into the AAV capsid protein. In one embodiment, the host cell is a HEK 293 cell. These methods are described in more detail in WO2017160360 A2, which is incorporated by reference herein. Methods of generating the capsid, coding sequences therefor, and methods for production of rAAV viral vectors have been described. See, e.g., Gao, et al, Proc. Natl. Acad. Sci. U.S.A. 100 (10), 6081-6086 (2003) and US 2013/0045186A1.

In one embodiment, a production cell culture useful for producing a recombinant AAV is provided. Such a cell culture contains a nucleic acid which expresses the AAV capsid protein in the host cell; a nucleic acid molecule suitable for packaging into the AAV capsid, e.g., a vector genome which contains AAV ITRs and a non-AAV nucleic acid sequence encoding a gene product operably linked to sequences which direct expression of the product in a host cell; and sufficient AAV rep functions and adenovirus helper functions to permit packaging of the nucleic acid molecule into the recombinant AAV capsid. In one embodiment, the cell culture is composed of mammalian cells (e.g., human embryonic kidney 293 cells, among others) or insect cells (e.g., baculovirus).

Typically, the rep functions are from the same AAV source as the AAV providing the ITRs flanking the vector genome. In the examples herein, the AAV2 ITRs are selected and the AAV2 rep is used. Optionally, other rep sequences or another rep source (and optionally another ITR source) may be selected. For example, the rep may be, but is not limited to, AAV1 rep protein, AAV2 rep protein; or rep 78, rep 68, rep 52, rep 40, rep68/78 and rep40/52; or a fragment thereof; or another source. Optionally, the rep and cap sequences are on the same genetic element in the cell culture. There may be a spacer between the rep sequence and cap gene. Any of these AAV or mutant AAV capsid sequences may be under the control of exogenous regulatory control sequences which direct expression thereof in a host cell.

In one embodiment, cells are manufactured in a suitable cell culture (e.g., HEK 293) cells. Methods for manufacturing the gene therapy vectors described herein include methods well known in the art such as generation of plasmid DNA used for production of the gene therapy vectors, generation of the vectors, and purification of the vectors. In some embodiments, the gene therapy vector is an AAV vector and the plasmids generated are an AAV cis-plasmid encoding the AAV genome and the gene of interest, an AAV trans-plasmid containing AAV rep and cap genes, and an adenovirus helper plasmid. The vector generation process can include method steps such as initiation of cell culture, passage of cells, seeding of cells, transfection of cells with the plasmid DNA, post-transfection medium exchange to serum free medium, and the harvest of vector-containing cells and culture media.

In certain embodiments, the manufacturing process for rAAV.hMfn2 involves transient transfection of HEK293 cells with plasmid DNA. A single batch or multiple batches are produced by PEI-mediated triple transfection of HEK293 cells in PALL iCELLis bioreactors. Harvested AAV material are purified sequentially by clarification, TFF, affinity chromatography, and anion exchange chromatography in disposable, closed bioprocessing systems where possible.

The harvested vector-containing cells and culture media are referred to herein as crude cell harvest. In yet another system, the gene therapy vectors are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, the contents of each of which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065, which are incorporated herein by reference.

The crude cell harvest may thereafter be subject to additional method steps such as concentration of the vector harvest, diafiltration of the vector harvest, microfluidization of the vector harvest, nuclease digestion of the vector harvest, filtration of microfluidized intermediate, crude purification by chromatography, crude purification by ultracentrifugation, buffer exchange by tangential flow filtration, and/or formulation and filtration to prepare bulk vector.

A two-step affinity chromatography purification at high salt concentration followed anion exchange resin chromatography are used to purify the vector drug product and to remove empty capsids. These methods are described in more detail in International Patent Application No. PCT/US2016/065970, filed Dec. 9, 2016, which is incorporated by reference herein. Purification methods for AAV8, International Patent Application No. PCT/US2016/065976, filed Dec. 9, 2016, and rh10, International Patent Application No. PCT/US16/66013, filed Dec. 9, 2016, entitled “Scalable Purification Method for AAVrh10”, also filed Dec. 11, 2015, and for AAV1, International Patent Application No. PCT/US2016/065974, filed Dec. 9, 2016, for “Scalable Purification Method for AAV1”, filed Dec. 11, 2015, are all incorporated by reference herein.

To calculate empty and full particle content, VP3 band volumes for a selected sample (e.g., in examples herein an iodixanol gradient-purified preparation where # of GC=# of particles) are plotted against GC particles loaded. The resulting linear equation (y=mx+c) is used to calculate the number of particles in the band volumes of the test article peaks. The number of particles (pt) per 20 μL loaded is then multiplied by 50 to give particles (pt) /mL. Pt/mL divided by GC/mL gives the ratio of particles to genome copies (pt/GC). Pt/mL-GC/mL gives empty pt/mL. Empty pt/mL divided by pt/mL and ×100 gives the percentage of empty particles.

Generally, methods for assaying for empty capsids and AAV vector particles with packaged genomes have been known in the art. See, e.g., Grimm et al., Gene Therapy (1999) 6:1322-1330; Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Virol. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer's instructions or other suitable staining method, i.e., SYPRO ruby or coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

In one aspect, an optimized q-PCR method is used which utilizes a broad-spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2-fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000-fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 April; 25(2):115-25. doi: 10.1089/hgtb.2013.131. Epub 2014 Feb. 14.

In brief, the method for separating rAAV particles having packaged genomic sequences from genome-deficient AAV intermediates involves subjecting a suspension comprising recombinant AAV viral particles and AAV capsid intermediates to fast performance liquid chromatography, wherein the AAV viral particles and AAV intermediates are bound to a strong anion exchange resin equilibrated at a high pH, and subjected to a salt gradient while monitoring eluate for ultraviolet absorbance at about 260 and about 280. The pH may be adjusted depending upon the AAV selected. See, e.g., WO2017/160360 (AAV9), WO2017/100704 (AAVrh10), WO 2017/100676 (e.g., AAV8), and WO 2017/100674 (AAV1), which are incorporated by reference herein. In this method, the AAV full capsids are collected from a fraction which is eluted when the ratio of A260/A280 reaches an inflection point. In one example, for the Affinity Chromatography step, the diafiltered product may be applied to a Capture Select™ Poros-AAV2/9 affinity resin (Life Technologies) that efficiently captures the AAV2 serotype. Under these ionic conditions, a significant percentage of residual cellular DNA and proteins flow through the column, while AAV particles are efficiently captured.

### Non-AAV and Non-Viral Vectors

A “vector” as used herein is a biological or chemical moiety comprising a nucleic acid sequence which can be introduced into an appropriate target cell for replication or expression of said nucleic acid sequence. Examples of a vector includes but not limited to a recombinant virus, a plasmid, Lipoplexes, a Polymersome, Polyplexes, a dendrimer, a cell penetrating peptide (CPP) conjugate, a magnetic particle, or a nanoparticle. In one embodiment, a vector is a nucleic acid molecule into which an exogenous or heterologous or engineered hMfn2 coding sequence (and/or at least one miRNA) may be inserted, which can then be introduced into an appropriate target cell. Such vectors preferably have one or more origin of replication, and one or more site into which the recombinant DNA can be inserted. Vectors often have means by which cells with vectors can be selected from those without, e.g., they encode drug resistance genes. Common vectors include plasmids, viral genomes, and “artificial chromosomes”. Conventional methods of generation, production, characterization or quantification of the vectors are available to one of skill in the art.

In one embodiment, the vector is a non-viral plasmid that comprises an expression cassette described thereof, e.g., “naked DNA”, “naked plasmid DNA”, RNA, mRNA, shRNA, RNAi, etc. Optionally the plasmid or other nucleic acid sequence is delivered via a suitable device, e.g., via electrospray, electroporation. In other embodiments, the nucleic acid molecule is coupled with various compositions and nano particles, including, e.g., micelles, liposomes, cationic lipid-nucleic acid compositions, poly-glycan compositions and other polymers, lipid and/or cholesterol-based—nucleic acid conjugates, and other constructs such as are described herein. See, e.g., WO2014/089486, US 2018/0353616A1, US2013/0037977A1, WO2015/074085A1, U.S. Pat. No. 9,670,152B2, and U.S. Pat. No. 8,853,377B2, X. Su et al, Mol. Pharmaceutics, 2011, 8 (3), pp 774-787; web publication: Mar. 21, 2011; WO2013/182683, WO 2010/053572 and WO 2012/170930, all of which are incorporated herein by reference.

In certain embodiment, a non-viral vector is used for delivery of an miRNA transcript targeting endogenous hMfn2 at a site not present in the co-administered engineered hMfn2 sequence. In some embodiments, the miRNA is delivered at an amount greater than about 0.5 mg/kg (e.g., greater than about 1.0 mg/kg, 1.5 mg/kg, 2.0 mg/kg, 2.5 mg/kg, 3.0 mg/kg, 4.0 mg/kg, 5.0 mg/kg, 6.0 mg/kg, 7.0 mg/kg, 8.0 mg/kg, 9.0 mg/kg, or 10.0 mg/kg) body weight of miRNA per dose. In some embodiments, the miRNA is delivered at an amount ranging from about 0.1-100 mg/kg (e.g., about 0.1-90 mg/kg, 0.1-80 mg/kg, 0.1-70 mg/kg, 0.1-60 mg/kg, 0.1-50 mg/kg, 0.1-40 mg/kg, 0.1-30 mg/kg, 0.1-20 mg/kg, 0.1-10 mg/kg) body weight of miRNA per dose. In some embodiments, the miRNA is delivered at an amount of or greater than about 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, or 500 mg per dose.

In certain embodiments, miRNA transcripts are encapsulated in a lipid nanoparticle (LNP). As used herein, the phrase “lipid nanoparticle” refers to a transfer vehicle comprising one or more lipids (e.g., cationic lipids, non-cationic lipids, and PEG-modified lipids). Preferably, the lipid nanoparticles are formulated to deliver one or more miRNA to one or more target cells (e.g., dorsal root ganglion, lower motor neurons and/or upper motor neurons, or the cell types identified above in the CNS). Examples of suitable lipids include, for example, the phosphatidyl compounds (e.g., phosphatidylglycerol, phosphatidylcholine, phosphatidylserine, phosphatidylethanolamine, sphingolipids, cerebrosides, and gangliosides). Also contemplated is the use of polymers as transfer vehicles, whether alone or in combination with other transfer vehicles. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, dendrimers and polyethylenimine. In one embodiment, the transfer vehicle is selected based upon its ability to facilitate the transfection of a miRNA to a target cell. Useful lipid nanoparticles for miRNA comprise a cationic lipid to encapsulate and/or enhance the delivery of miRNA into the target cell that will act as a depot for protein production. As used herein, the phrase “cationic lipid” refers to any of a number of lipid species that carry a net positive charge at a selected pH, such as physiological pH. The contemplated lipid nanoparticles may be prepared by including multi-component lipid mixtures of varying ratios employing one or more cationic lipids, non-cationic lipids and PEG-modified lipids. Several cationic lipids have been described in the literature, many of which are commercially available. See, e.g., WO2014/089486, US 2018/0353616A1, and U.S. Pat. No. 8,853,377B2, which are incorporated by reference. In certain embodiments, LNP formulation is performed using routine procedures comprising cholesterol, ionizable lipid, helper lipid, PEG-lipid and polymer forming a lipid bilayer around encapsulated mRNA (Kowalski et al., 2019, Mol. Ther. 27(4):710-728). In some embodiments, LNP comprises a cationic lipids (i.e. N-[1-(2,3-dioleoyloxy)propyl]-N,N,N-trimethylammonium chloride (DOTMA), or 1,2-dioleoyl-3-trimethylammonium-propane (DOTAP)) with helper lipid DOPE. In some embodiments, LNP comprises an ionizable lipid Dlin-MC3-DMA ionizable lipids, or diketopiperazine-based ionizable lipids (cKK-E12). In some embodiments, polymer comprises a polyethyleneimine (PEI), or a poly(β-amino)esters (PBAEs). See, e.g., WO2014/089486, US 2018/0353616A1, US2013/0037977A1, WO2015/074085A1, U.S. Pat. No. 9,670,152B2, and U.S. Pat. No. 8,853,377B2, which are incorporated by reference.

In certain embodiments, the vector described herein is a “replication-defective virus” or a “viral vector” which refers to a synthetic or artificial viral particle in which an expression cassette containing a nucleic acid sequence encoding an engineered hMfn2 and/or at least one miRNA targeting endogenous hMfn2 at a site not present on the sequence of the engineered hMfn2. Replication-defective viruses cannot generate progeny virions but retain the ability to infect target cells. In one embodiment, the genome of the viral vector does not include genes encoding the enzymes required to replicate (the genome can be engineered to be “gutless”-containing only the nucleic acid sequence encoding E2 flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

As used herein, a recombinant viral vector may be any suitable replication-defective viral vector, including, e.g., a recombinant adeno-associated virus (AAV), an adenovirus, a bocavirus, a hybrid AAV/bocavirus, a herpes simplex virus or a lentivirus.

As used herein, the term “host cell” may refer to the packaging cell line in which a vector (e.g., a recombinant AAV) is produced. A host cell may be a prokaryotic or eukaryotic cell (e.g., human, insect, or yeast) that contains exogenous or heterologous DNA that has been introduced into the cell by any means, e.g., electroporation, calcium phosphate precipitation, microinjection, transformation, viral infection, transfection, liposome delivery, membrane fusion techniques, high velocity DNA-coated pellets, viral infection and protoplast fusion. Examples of host cells may include, but are not limited to an isolated cell, a cell culture, an Escherichia coli cell, a yeast cell, a human cell, a non-human cell, a mammalian cell, a non-mammalian cell, an insect cell, an HEK-293 cell, a liver cell, a kidney cell, a cell of the central nervous system, a neuron, a glial cell, or a stem cell.

As used herein, the term “target cell” refers to any target cell in which expression of the hMfn2 and/or miRNA is desired. In certain embodiments, the term “target cell” is intended to reference the cells of the subject being treated for CMT2A. Examples of target cells may include, but are not limited to, cells within the central nervous system.

### Compositions

Provided herein are compositions containing at least one vector comprising hMfn2.miR (e.g., an rAAV.hMfn2.miR stock) and/or at least one vector comprising miR and/or at least one vector comprising stock, and an optional carrier, excipient and/or preservative.

As used herein, a “stock” of rAAV refers to a population of rAAV. Despite heterogeneity in their capsid proteins due to deamidation, rAAV in a stock are expected to 5 share an identical vector genome. A stock can include rAAV having capsids with, for example, heterogeneous deamidation patterns characteristic of the selected AAV capsid proteins and a selected production system. The stock may be produced from a single production system or pooled from multiple runs of the production system. A variety of production systems, including but not limited to those described herein, may be selected.

In certain embodiments, a composition comprises at least virus stock which is a recombinant AAV (rAAV) suitable for use in treating CMT2A alone or in combination with other vector stock or composition. In certain embodiments, the composition is suitable for use in preparing a medicament for treating CMT2A. In certain embodiments, a composition comprises a virus stock which is a recombinant AAV (rAAV) suitable for use in treating CMT2A, said rAAV comprising: (a) an adeno-associated virus capsid, and (b) a vector genome packaged in the AAV capsid, said vector genome comprising AAV inverted terminal repeats, a coding sequence for an engineered human mitofusin 2, a spacer sequence, a coding sequence for at least one miRNA specifically targeted to endogenous human mitofusin at a site not present in the engineered human mitofusin coding sequence, and regulatory sequences which direct expression of the encoded gene products. In certain embodiments, a composition comprises separate vector stock comprising rAAV comprising: (a) an adeno-associated virus capsid, and (b) a vector genome packaged in the AAV capsid, said vector genome comprising AAV inverted terminal repeats, a coding sequence for an engineered human mitofusin 2, and regulatory sequences which direct expression of the encoded gene product and/or a separate vector stock comprising (a) an adeno-associated virus capsid, and (b) a vector genome packaged in the AAV capsid, said vector genome comprising AAV inverted terminal repeats, a coding sequence for at least one miRNA specifically targeted to endogenous human mitofusin 2 at a site not present in the engineered human mitofusin 2 coding sequence, and regulatory sequences which direct expression of the encoded gene product. In certain embodiments, the vector genome comprises a promoter, an enhancer, an intron, a human Mfn2 coding sequence, and a polyadenylation signal. In certain embodiments, the intron consists of a chicken beta actin splice donor and a rabbit β splice acceptor element. In certain embodiments, the vector genome further comprises an AAV2 5′ ITR and an AAV2 3′ ITR which flank all elements of the vector genome.

The rAAV.hMfn2.miR (rAAV.hMfn2 or another vector) may be suspended in a physiologically compatible carrier to be administered to a human CMT2A patient. In certain embodiments, for administration to a human patient, the vector is suitably suspended in an aqueous solution containing saline, a surfactant, and a physiologically compatible salt or mixture of salts. Suitably, the formulation is adjusted to a physiologically acceptable pH, e.g., in the range of pH 6 to 9, or pH 6.5 to 7.5, pH 7.0 to 7.7, or pH 7.2 to 7.8. As the pH of the cerebrospinal fluid is about 7.28 to about 7.32, or a pH of 7.2 to 7.4, for intrathecal delivery, a pH within this range may be desired; whereas for intravenous delivery, a pH of about 6.8 to about 7.2 may be desired. However, other pHs within the broadest ranges and these subranges may be selected for other route of delivery.

In certain embodiments, the formulation may contain a buffered saline aqueous solution not comprising sodium bicarbonate. Such a formulation may contain a buffered saline aqueous solution comprising one or more of sodium phosphate, sodium chloride, potassium chloride, calcium chloride, magnesium chloride and mixtures thereof, in water, such as a Harvard's buffer. The aqueous solution may further contain Kolliphor® P188, a poloxamer which is commercially available from BASF which was formerly sold under the trade name Lutrol® F68. The aqueous solution may have a pH of 7.2 or a pH of 7.4.

In another embodiment, the formulation may contain a buffered saline aqueous solution comprising 1 mM Sodium Phosphate (Na3PO4), 150 mM sodium chloride (NaCl), 3 mM potassium chloride (KCl), 1.4 mM calcium chloride (CaCl2), 0.8 mM magnesium chloride (MgCl2), and 0.001% Kolliphor® 188. See, e.g., harvardapparatus.com/harvard-apparatus-perfusion-fluid.html. In certain embodiments, Harvard's buffer is preferred.

In other embodiments, the formulation may contain one or more permeation enhancers. Examples of suitable permeation enhancers may include, e.g., mannitol, sodium glycocholate, sodium taurocholate, sodium deoxycholate, sodium salicylate, sodium caprylate, sodium caprate, sodium lauryl sulfate, polyoxyethylene-9-laurel ether, or EDTA.

In another embodiment, the composition includes a carrier, diluent, excipient and/or adjuvant. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The buffer/carrier should include a component that prevents the rAAV, from sticking to the infusion tubing but does not interfere with the rAAV binding activity in vivo.

Optionally, the compositions may contain, in addition to the vector (e.g., rAAV) and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically acceptable” refers to molecular entities and compositions that do not produce an allergic or similar untoward reaction when administered to a host. Delivery vehicles such as liposomes, nanocapsules, microparticles, microspheres, lipid particles, vesicles, and the like, may be used for the introduction of the compositions of the present invention into suitable host cells. In particular, the rAAV vector delivered transgenes may be formulated for delivery either encapsulated in a lipid particle, a liposome, a vesicle, a nanosphere, or a nanoparticle or the like.

In one embodiment, a composition includes a final formulation suitable for delivery to a subject, e.g., is an aqueous liquid suspension buffered to a physiologically compatible pH and salt concentration. Optionally, one or more surfactants are present in the formulation. In another embodiment, the composition may be transported as a concentrate which is diluted for administration to a subject. In other embodiments, the composition may be lyophilized and reconstituted at the time of administration.

A suitable surfactant, or combination of surfactants, may be selected from among nonionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits ×100 give the approximate molecular mass of the polyoxypropylene core, and the last digit ×10 gives the percentage polyoxyethylene content. In one embodiment Poloxamer 188 is selected. The surfactant may be present in an amount up to about 0.0005% to about 0.001% of the suspension.

The vectors are administered in sufficient amounts to transfect the cells and to provide sufficient levels of gene transfer and expression to provide a therapeutic benefit without undue adverse effects, or with medically acceptable physiological effects, which can be determined by those skilled in the medical arts. Optionally, routes other than intrathecal administration may be used, such as, e.g., direct delivery to a desired organ (e.g., the liver (optionally via the hepatic artery), lung, heart, eye, kidney), oral, inhalation, intranasal, intratracheal, intraarterial, intraocular, intravenous, intramuscular, subcutaneous, intradermal, and other parental routes of administration. Routes of administration may be combined, if desired.

Dosages of the vector will depend primarily on factors such as the condition being treated, the age, weight and health of the patient, and may thus vary among patients. For example, a therapeutically effective human dosage of viral vector is generally in the range of from about 25 to about 1000 microliters to about 100 mL of solution containing concentrations of from about 1×10⁹ to 1×10¹⁶ genomes virus vector (to treat an average subject of 70 kg in body weight) including all integers or fractional amounts within the range, and preferably 1.0×10¹² GC to 1.0×10¹⁴ GC for a human patient. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 1×10¹⁰ to about 1×10¹² GC per dose including all integers or fractional amounts within the range.

In certain embodiments, the dose is in the range of about 1×10⁹ GC/g brain mass to about 1×10¹² GC/g brain mass. In certain embodiments, the dose is in the range of about 1×10¹⁰ GC/g brain mass to about 3.33×10¹¹ GC/g brain mass. In certain embodiments, the dose is in the range of about 3.33×10¹¹ GC/g brain mass to about 1.1×10¹² GC/g brain mass. In certain embodiments, the dose is in the range of about 1.1×10¹² GC/g brain mass to about 3.33×10¹³ GC/g brain mass. In certain embodiments, the dose is lower than 3.33×10¹¹ GC/g brain mass. In certain embodiments, the dose is lower than 1.1×10¹² GC/g brain mass. In certain embodiments, the dose is lower than 3.33×10¹³ GC/g brain mass. In certain embodiments, the dose is about 1×10¹⁰ GC/g brain mass. In certain embodiments, the dose is about 2×10¹⁰ GC/g brain mass. In certain embodiments, the dose is about 2×10¹⁰ GC/g brain mass. In certain embodiments, the dose is about 3×10¹⁰ GC/g brain mass. In certain embodiments, the dose is about 4×10¹⁰ GC/g brain mass. In certain embodiments, the dose is about 5×10¹⁰ GC/g brain mass. In certain embodiments, the dose about 6×10¹⁰ GC/g brain mass. In certain embodiments, the dose is about 7×10¹⁰ GC/g brain mass. In certain embodiments, the dose about 8×10¹⁰ GC/g brain mass. In certain embodiments, the dose is about 9×10¹⁰ GC/g brain mass. In certain embodiments, the dose is about 1×10¹¹ GC/g brain mass. In certain embodiments, the dose is about 2×10¹¹ GC/g brain mass. In certain embodiments, the dose is about 3×10¹¹ GC/g brain mass. In certain embodiments, the dose is about 4×10¹¹ GC/g brain mass. In certain embodiments, the dose is administered to humans as a flat dose in the range of about 1.44×10¹³ to 4.33×10¹⁴ GC of the rAAV. In certain embodiments, the dose is administered to humans as a flat dose in the range of about 1.44×10¹³ to 2×10¹⁴ GC of the rAAV. In certain embodiments, the dose is administered to humans as a flat dose in the range of about 3×10¹³ to 1×10¹⁴ GC of the rAAV. In certain embodiments, the dose is administered to humans as a flat dose in the range of about 5×10¹³ to 1×10¹⁴ GC of the rAAV. In some embodiments, the compositions can be formulated in dosage units to contain an amount of AAV that is in the range of about 1×10¹³ to 8×10¹⁴ GC of the rAAV. In some embodiments, the compositions can be formulated in dosage units to contain an amount of rAAV that is in the range of about 1.44×10¹³ to 4.33×10¹⁴ GC of the rAAV. In some embodiments, the compositions can be formulated in dosage units to contain an amount of rAAV that is in the range of about 3×10¹³ to 1×10¹⁴ GC of the rAAV. In some embodiments, the compositions can be formulated in dosage units to contain an amount of rAAV that is in the range of about 5×10¹³ to 1×10¹⁴ GC of the rAAV.

In certain embodiments, the vector is administered to a subject in a single dose. In certain embodiments, vector may be delivered via multiple injections (for example 2 doses) is desired.

The dosage will be adjusted to balance the therapeutic benefit against any side effects and such dosages may vary depending upon the therapeutic application for which the recombinant vector is employed. The levels of expression of the transgene can be monitored to determine the frequency of dosage resulting in viral vectors, preferably AAV vectors containing the minigene. Optionally, dosage regimens similar to those described for therapeutic purposes may be utilized for immunization using the compositions provided herein.

As used herein, the terms “intrathecal delivery” or “intrathecal administration” refer to a route of administration via an injection into the spinal canal, more specifically into the subarachnoid space so that it reaches the cerebrospinal fluid (CSF). Intrathecal delivery may include lumbar puncture, intraventricular (including intracerebroventricular (ICV)), suboccipital/intracisternal, and/or C1-2 puncture. For example, material may be introduced for diffusion throughout the subarachnoid space by means of lumbar puncture. In another example, injection may be into the cisterna magna.

As used herein, the terms “intracisternal delivery” or “intracisternal administration” refer to a route of administration directly into the cerebrospinal fluid of the cisterna magna cerebellomedularis, more specifically via a suboccipital puncture or by direct injection into the cisterna magna or via permanently positioned tube.

Compositions comprising the miR target sequences described herein for repressing endogenous hMfn2 (e.g., in CMT2A patients) are generally targeted to one or more different cell types within the central nervous system, including, but not limited to, neurons (including, e.g., lower motor neurons and/or primary sensory neurons. These may include, e.g., pyramidal, purkinje, granule, spindle, and interneuron cells).

### Uses

The vectors and compositions provided herein are useful for treating a patient having Charcot-Marie-Tooth (CMT) disorder, neuropathy, or various symptoms associated therewith. A combination regimen or co-therapy for treating a patient having CMT2A is provided. In certain embodiments, this regimen or co-therapy comprises co-administering (a) a recombinant nucleic acid sequence encoding an engineered human mitofusin 2 coding sequence operably linked to regulatory sequences which direct expression thereof in a human target cell, wherein the human mitofusin 2 coding sequence has the sequence of SEQ ID NO: 15 or a sequence at least 95% identical thereto and which differs from endogenous human mitofusin 2 in the CMT2A patient by having a mismatch in the miRNA target sequence of (b), and (b) a coding sequence for at least one miRNA specific for an endogenous human mitofusin 2 sequence in a human CMT2A subject, wherein the mRNA is operably linked to regulatory sequences which direct expression thereof in the subject.

In certain embodiments, this regimen or co-therapy comprises co-administering (a) a recombinant nucleic acid sequence encoding an engineered human mitofusin 2 coding sequence operably linked to regulatory sequences which direct expression thereof in a human target cell, wherein the engineered human mitofusin 2 coding sequence has the sequence of SEQ ID NO: 11 or a sequence at least 95% identical thereto and which differs from endogenous human mitofusin 2 in the CMT2A patient by having a mismatch in the miRNA target sequence of (b), and (b) a coding sequence for at least one miRNA specific for an endogenous human mitofusin 2 sequence in a human CMT2A subject, wherein the at least one miRNA coding sequence is operably linked to regulatory sequences which direct expression thereof in the subject, and wherein the at least one miRNA coding sequence comprises a sequence of one or more of an miRNA targeting sequence comprising SEQ ID NO: 89 (miR538, 59 nt) or an miRNA comprising one or more of antisense sequence of SEQ ID NO: 27.

In certain embodiments, this regimen or co-therapy for treating a patient having CMT2A comprises co-administering (a) a recombinant nucleic acid sequence encoding an engineered human mitofusin 2 coding sequence operably linked to regulatory sequences which direct expression thereof in a human target cell, wherein the human mitofusin 2 coding sequence is engineered to differs from endogenous human mitofusin 2 in the CMT2A patient by having a mismatch in the miRNA target sequence of (b), and (b) a coding sequence for at least one miRNA specific for an endogenous human mitofusin 2 sequence in a human CMT2A subject, wherein the miRNA coding sequence is operably linked to regulatory sequences which direct expression thereof in the subject, and wherein the at least one miRNA coding sequence has a sequence of one or more of: an miRNA coding sequence comprising SEQ ID NO: 15 (miR1693, 64 nt); an miRNA coding sequence comprising at least 60 consecutive nucleotides of SEQ ID NO: 15; an miRNA coding sequence comprising at least 99% identity to SEQ ID NO: 15 which comprises a sequence with 100% identity to about nucleotide 6 to about nucleotide 26 of SEQ ID NO: 15 (or SEQ ID NO: 68); or an miRNA coding sequence comprising one or more of:

(i) TTGACGTCCAGAACCTGTTCT, SEQ ID NO: 27; (ii) AGAAGTGGGCACTTAGAGTTG, SEQ ID NO: 28; (iii) TTCAGAAGTGGGCACTTAGAG, SEQ ID NO: 29; (iv) TTGTCAATCCAGCTGTCCAGC, SEQ ID NO: 30; (v) CAAACTTGGTCTTCACTGCAG, SEQ ID NO: 31; (vi) AAACCTTGAGGACTACTGGAG, SEQ ID NO: 32; (vii) TAACCATGGAAACCATGAACT, SEQ ID NO: 33; (viii) ACAACAAGAATGCCCATGGAG, SEQ ID NO: 34; (ix) AAAGGTCCCAGACAGTTCCTG, SEQ ID NO: 35; (x) TGTTCATGGCGGCAATTTCCT, SEQ ID NO: 36; (xi) TGAGGTTGGCTATTGATTGAC, SEQ ID NO: 37; (xii) TTCTCACACAGTCAACACCTT, SEQ ID NO: 38; (xiii) TTTCCTCGCAGTAAACCTGCT, SEQ ID NO: 39; (xiv) AGAAATGGAACTCAATGTCTT, SEQ ID NO: 40; (xv) TGAACAGGACATCACCTGTGA, SEQ ID NO: 41; (xvi) AATACAAGCAGGTATGTGAAC, SEQ ID NO: 42; (xvii) TAAACCTGCTGCTCCCGAGCC, SEQ ID NO: 43; (xviii) TAGAGGAGGCCATAGAGCCCA, SEQ ID NO: 44; (xix) TCTACCCGCAGGAAGCAATTG, SEQ ID NO: 45; or (xx) CTCCTTAGCAGACACAAAGAA, SEQ ID NO: 46, or combinations of any of (i) through (xx). In certain embodiments, a first vector comprises the nucleic acid (a) and a second, different vector, comprises at least one miRNA (b). In certain embodiments, the first vector is a viral vector and/or the second vector is a viral vector and the first and the second viral vector may be from the same virus source or may be different. In certain embodiments, the first vector is a non-viral vector, the second vector is a non-viral vector and the first and the second vectors may be same composition or may be different.

In certain embodiments, the vectors and compositions provided herein are useful for treating patients having Mfn2-induced lipomatosis. In certain embodiments. the vectors and compositions provided herein are useful for treating patients having multiple symmetric lipomatosis. Multiple symmetric lipomatosis is associated with rare genetic mutation in Mfn2 gene, and characterizable by large deposits and accumulate of fat tissue in upper bodies and gradually lose fat tissue in arms and legs (Rocha, N., et al., Human biallelic MFN2 mutations induce mitochondrial dysfunction, upper body adipose hyperplasia, and suppression of leptin expression, eLife, 2017, 6:1-27, Apr. 19, 2017). In certain embodiments. the vectors and compositions provided herein are useful for treating patients having severe early-onset neuropathy due to Mfn2 deficiency. In certain embodiments, the vectors and compositions provided herein are useful for treating patients having Alzheimer's Disease (AD), Parkinson's Disease (PD), cardiomyopathies, and Mfn2-associated pathogenesis in various cancers. It has been evidenced that there is a suggested link between Mfn2 deregulation and AD and PD, e.g., link between single nucleotide polymorphism in the Mfn2 gene and AD risk (Filadi, R., et al., Cell Death and Disease, 2018, 9:330).

Optionally, the vectors and compositions provided herein may be used in combination with one or more co-therapies selected from: acetaminophen, nonsteroidal anti-inflammatory drugs (NSAIDs), tricyclic antidepressants or antiepileptic drugs, such as carbamazepine or gabapentin. In still other embodiments, the vectors may be delivered in a combination with an immunomodulatory regimen involving one or more steroids, e.g., prednisone.

As used herein, the term Computed Tomography (CT) refers to radiography in which a three-dimensional image of a body structure is constructed by computer from a series of plane cross-sectional images made along an axis.

The term “substantial homology” or “substantial similarity,” when referring to a nucleic acid, or fragment thereof, indicates that, when optimally aligned with appropriate nucleotide insertions or deletions with another nucleic acid (or its complementary strand), there is nucleotide sequence identity in at least about 95 to 99% of the aligned sequences. Preferably, the homology is over full-length sequence, or an open reading frame thereof, or another suitable fragment which is at least 15 nucleotides in length. Examples of suitable fragments are described herein.

The terms “sequence identity” “percent sequence identity” or “percent identical” in the context of nucleic acid sequences refers to the residues in the two sequences which are the same when aligned for maximum correspondence. The length of sequence identity comparison may be over the full-length of the genome, the full-length of a gene coding sequence, or a fragment of at least about 500 to 5000 nucleotides, is desired. However, identity among smaller fragments, e.g., of at least about nine nucleotides, usually at least about to 24 nucleotides, at least about 28 to 32 nucleotides, at least about 36 or more nucleotides, may also be desired. Similarly, “percent sequence identity” may be readily determined for amino acid sequences, over the full-length of a protein, or a fragment thereof. Suitably, a fragment is at least about 8 amino acids in length and may be up to about 700 amino acids. Examples of suitable fragments are described herein.

By the term “highly conserved” is meant at least 80% identity, preferably at least 90% identity, and more preferably, over 97% identity. Identity is readily determined by one of skill in the art by resort to algorithms and computer programs known by those of skill in the art.

Unless otherwise specified by an upper range, it will be understood that a percentage of identity is a minimum level of identity and encompasses all higher levels of identity up to 100% identity to the reference sequence. Unless otherwise specified, it will be understood that a percentage of identity is a minimum level of identity and encompasses all higher levels of identity up to 100% identity to the reference sequence. For example, “95% identity” and “at least 95% identity” may be used interchangeably and include 95, 96, 97, 98, 99 up to 100% identity to the referenced sequence, and all fractions therebetween.

Unless otherwise specified, numerical values will be understood to be subject to conventional mathematic rounding rules.

Generally, when referring to “identity”, “homology”, or “similarity” between two different adeno-associated viruses, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. In the examples, AAV alignments are performed using the published AAV9 sequences as a reference point. Alignments are performed using any of a variety of publicly or commercially available Multiple Sequence Alignment Programs. Examples of such programs include, “Clustal Omega”, “Clustal W”, “CAP Sequence Assembly”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference. Multiple sequence alignment programs are also available for amino acid sequences, e.g., the “Clustal Omega”, “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

It is to be noted that the term “a” or “an” refers to one or more. As such, the terms “a (or “an”), “one or more,” and “at least one” are used interchangeably herein.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

As used herein, the term “about” means a variability of 10% (±10%, e.g., ±1, ±2, ±3, ±4, ±5, ±6, ±7, ±8, ±9, ±10, or values therebetween) from the reference given, unless otherwise specified.

As used herein, “disease”, “disorder” and “condition” are used interchangeably, to indicate an abnormal state in a subject.

As used herein, the term “CMT2A-related symptom(s)” or “symptom(s)” refers to symptom(s) found in CMT2A patients as well as in CMT2A animal models. Early symptoms of CMT may include one or more of clumsiness, slight difficulty in walking because of trouble picking up the feet, weak leg muscles, fatigue, absence of reflexes. Common symptoms of CMT2A include, foot deformity (very high arched foot/feet), difficulty lifting foot at the ankle (foot drop), curled toes (known as hammer toes), loss of lower leg muscle, which leads to skinny calves, numbness or burning sensation in the feet or hands, “Slapping” when walking (feet hit the floor hard when walking), weakness of the hips, legs, or feet, leg and hand cramps, Loss of balance, tripping, and falling, difficulty grasping and holding objects and opening jars and bottles, pain (both nerve pain and arthritic pain). Later symptoms of CMT2A may include, e.g., a similar symptoms in the arms and hands, curvature of the spine (scoliosis). Other reported/known symptoms of CMT2A may include, e.g., speech and swallowing difficulties, breathing difficulties, especially when lying flat, hearing loss, vision loss, vocal cord paralysis.

“Patient” or “subject” as used herein means a male or female human, and animal models (including, e.g., dogs, non-human primates, rodents, or other suitable models) used for clinical research. In one embodiment, the subject of these methods and compositions is a human diagnosed with CMT2A. In certain embodiments, the human subject of these methods and compositions is a prenatal, a newborn, an infant, a toddler, a preschool, a grade-schooler, a teen, a young adult or an adult. In a further embodiment, the subject of these methods and compositions is a pediatric CTM2A patient.

As used herein, the term “a therapeutic level” means an Mfn2 activity at least about 5%, about 8%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, more than 100%, about 2-fold, about 3-fold, or about 5-fold of a healthy control. Suitable assays for measuring the activity of an hMfn2 are known in the art. In some embodiments, such therapeutic levels of the one or more subunit protein may result in alleviation of the CMT2A related symptom(s); reversal of certain CMT2A-related symptoms and/or prevention of progression of CMT2A-related certain symptoms; or any combination thereof. In certain embodiments, the human Mfn2 delivered by the compositions and regimens provided herein has the amino acid sequence of a functional endogenous wild-type protein. In certain embodiments, the sequence is the amino acid sequence of SEQ ID NO: 19 or a functional protein which is at about 95 to 100% identity to functional, human Mfn2 protein.

The term “expression” is used herein in its broadest meaning and comprises the production of RNA or of RNA and protein. With respect to RNA, the term “expression” or “translation” relates in particular to the production of peptides or proteins. Expression may be transient or may be stable.

Additionally optionally, an expression cassette (and a vector genome) may comprise one or more dorsal root ganglion (drg)-miRNA targeting sequences in the UTR, e.g., to reduce drg toxicity and/or axonopathy. See, e.g., PCT/US2019/67872, filed Dec. 20, 2019 and now published as WO 2020/132455, U.S. Provisional Patent Application No. 63/023,593, filed May 12, 2020, and U.S. Provisional Patent Application No. 63/038,488, filed Jun. 12, 2020, all entitled “Compositions for Drg-Specific Reduction of Transgene Expression”, which are incorporated herein in their entireties. In some embodiments, an expression cassette may be delivered via a genetic element (e.g., a plasmid) to a packaging host cell and packaged into the capsid of a viral vector (e.g., a viral particle).

As used herein, the term “operably linked” refers to both expression control sequences that are contiguous with the gene of interest and expression control sequences that act in trans or at a distance to control the gene of interest.

The term “heterologous” when used with reference to a protein or a nucleic acid indicates that the protein or the nucleic acid comprises two or more sequences or subsequences which are not found in the same relationship to each other in nature. For instance, the nucleic acid is typically recombinantly produced, having two or more sequences from unrelated genes arranged to make a new functional nucleic acid. For example, in one embodiment, the nucleic acid has a promoter from one gene arranged to direct the expression of a coding sequence from a different gene. Thus, with reference to the coding sequence, the promoter is heterologous.

As described herein, regulatory elements comprise but not limited to: promoter; enhancer; transcription factor; transcription terminator; efficient RNA processing signals such as splicing and polyadenylation signals (polyA); sequences that stabilize cytoplasmic mRNA, for example Woodchuck Hepatitis Virus (WHP) Posttranscriptional Regulatory Element (WPRE); sequences that enhance translation efficiency (i.e., Kozak consensus sequence).

The term “translation” in the context of the present invention relates to a process at the ribosome, wherein an mRNA strand controls the assembly of an amino acid sequence to generate a protein or a peptide.

Expression cassettes containing engineered hMfn2 coding sequences are provided herein, e.g., Syn.PI.hMfn2eng.link.hMfn2.miR1693.WPRE.bGH (nt 223 to 4455 of SEQ ID NO: 1); CB7.CI.hMfn2 GA.RBG (nt 259 to 4370 of SEQ ID NO: 79); CB7.CI.hMfn2.GA.LINK.miR1518.RBG (nt 259 to 4710 of SEQ ID NO: 77); CB7.CI.hMfn2.GA.LINK.miR538.RBG (nt 259 to 4626 of SEQ ID NO: 75); CAG.CI.hMfn2 GA.WPRE.SV40 (nt 192 to 4262 of SEQ ID NO: 73); CAG.CI.hMfn2 GA.LINK.miR1518.WPRE.SV40 (nt 192 to 4474 of SEQ ID NO: 71); and CAG.CI.hMfn2.GA.LINK.miR538.WPRE.SV40 (nt 192 to 4474 of SEQ ID NO: 69).

Expression cassettes containing engineered rMfn2 coding sequences are provided herein, e.g., Syn.PI.rMnf2eng.link rMfn2 miR1518 WPRE.BGH (nt 223 to 4430 of SEQ ID NO: 3).

Unless defined otherwise in this specification, technical and scientific terms used herein have the same meaning as commonly understood by one of ordinary skill in the art and by reference to published texts, which provide one skilled in the art with a general guide to many of the terms used in the present application.

The following examples are illustrative only and are not intended to limit the present invention.

### EXAMPLES

The following examples are illustrative and are not intended to limit the present invention.

Charcot-Marie-Tooth neuropathy type 2A (CMT2A) is caused by mutations in the gene encoding mitofusin 2 (Mfn2), a protein required for mitochondrial fusion and transport along axons. In most cases of CMT2A is caused by autosomal dominant missense mutations that act in a dominant-negative manner. In rare cases, CMT2A is caused by recessive loss-of-function mutations. A Mfn2 mutations are selectively toxic to lower motor neurons and primary sensory neurons in dorsal root ganglia (DRG).

A gene therapy was developed to restore mitofusin-2 expression by overexpression of Mfn2 to overcome dominant negative activity of mutant Mfn2. An AAV gene therapy technology is used to transfer an Mfn2 expression cassette into neurons. In some embodiments an Mfn2 expression cassette contains a miRNA connected via a linker, wherein miRNA is targeted to knock down mutant (defective) Mfn2, to eliminate mutant Mfn2 and supply with normal mitofurin-2 protein. The injection of the therapeutic article is delivered via intra-cisterna magna (ICM), or intravenously. ICM delivery of AAV efficiently targets lower motor neurons and primary sensor neurons. A Mfn2 expression cassette, with engineered Mfn2 gene, highly optimized for expression in humans was used in combination with an AAV capsid with highly improved central nervous system (CNS) transduction.

### Example 1: CMT2A Vector Strategy

In an AAV transgene cassette strategy 1, we generated a Mfn expression cassette comprising a promoter, an intron, an engineered Mfn2 cDNA and polyA. In AAV transgene cassette strategy 2, we generated a Mfn2 expression cassette comprising a promoter, an intron, an engineered Mfn2 cDNA, which was engineered to optimize for expression and resistant to the miRNA, an miRNA targeting endogenous mutant Mfn2 and a polyA. Additionally, in an AAV transgene strategy 2, a construct was identified for efficient expression of a miRNA and cDNA in vivo, wherein the construct comprised a synapsin promoter, a cDNA (engineered Mfn2), a linker, a miRNA, wherein a miRNA located at the 3′ end of cDNA, a WRPE enhancer, and polyA (data not shown).

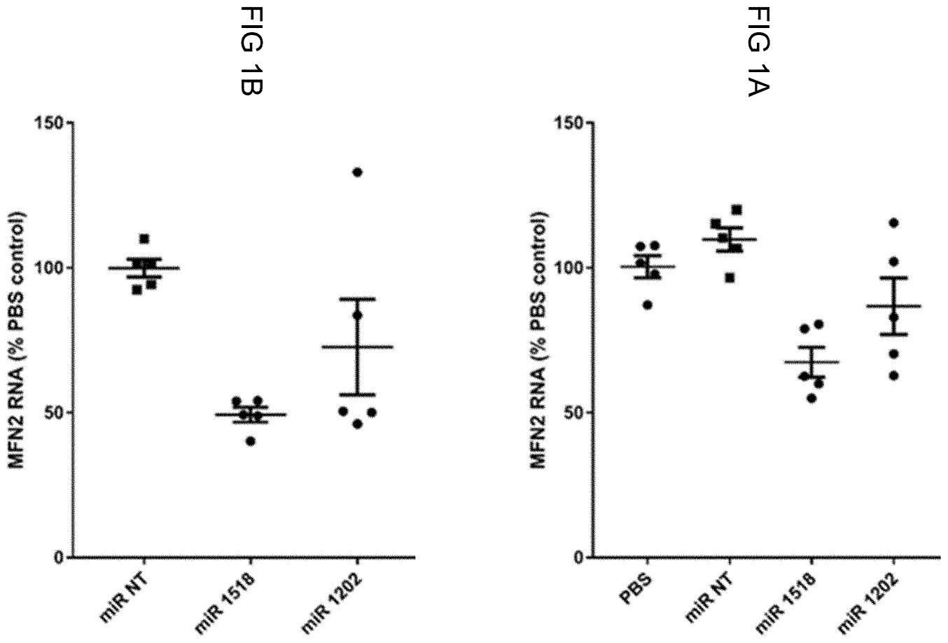

To knock down endogenous Mfn2 gene, various miRNA sequences various miRNA sequences were examined: miR238, miR1202, miR1518, and miR2282, among other, data for which is not shown, were examined. For western blot analysis, rat B104 cells were transfected with in vitro Block-iT plasmids (ThermoFisher) containing a CMV promoter. emGFP transgene, a cloning site for miRNA and TK polyA. miRNAs were designed using Block-iT online software. Cells were transfected and selected for expression with blasticidin. Surviving cells were mostly positive for GFP and overall transfection efficiency could be determined. B104 cells were harvested after 72 hours and prepared for western blot analysis. Western blot was probed with antibodies against Mfn2 and b-actin as loading control. FIG. 1A and FIG. 1B illustrate knockdown of endogenous Mfn2 RNA, as measured by qPCR, in mouse brain (FIG. 1A) and spinal cord (FIG. 1B) in B6 mice following intravenous administration of AAV-mediated delivery of miRNA. Mice were necropsied at day 14 post administration, following which brain and spinal cord tissues were harvested and homogenized. RNA was extracted from the samples and used for qPCR with TaqMan primers against Mfn2. Four treatment groups were examined (N=5 per group): PBS, AAV9.PHP.eB.CB7.miR_NT (non-targeted at a dose of 3×10¹¹ GC), AAV9.PHP.eB.CB7.miR_1518 (at a dose of 3×10¹¹ GC), and AAV9.PHP.eB.CB7.miR_1202 (at a dose of 3×10¹¹ GC). Among the sequences examined, miR1518 illustrated best knockdown of endogenous Mfn2.

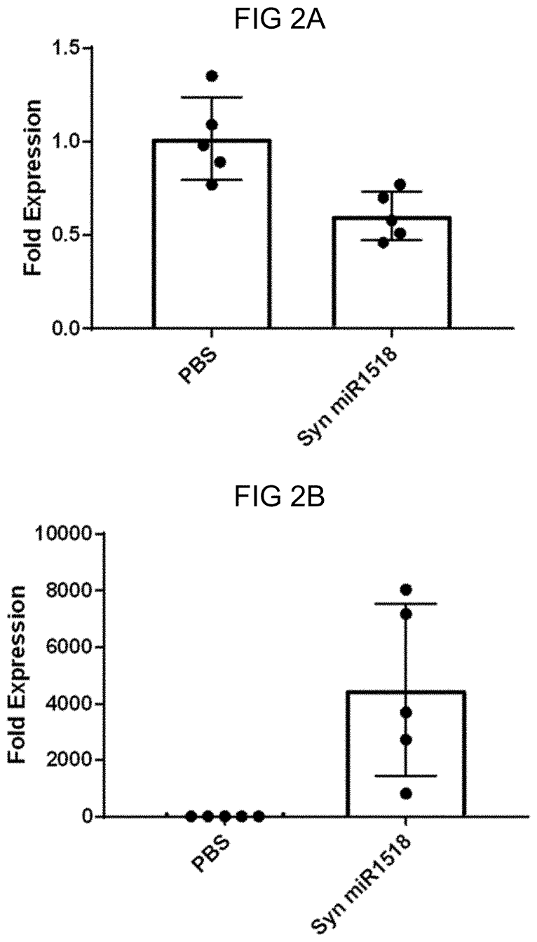

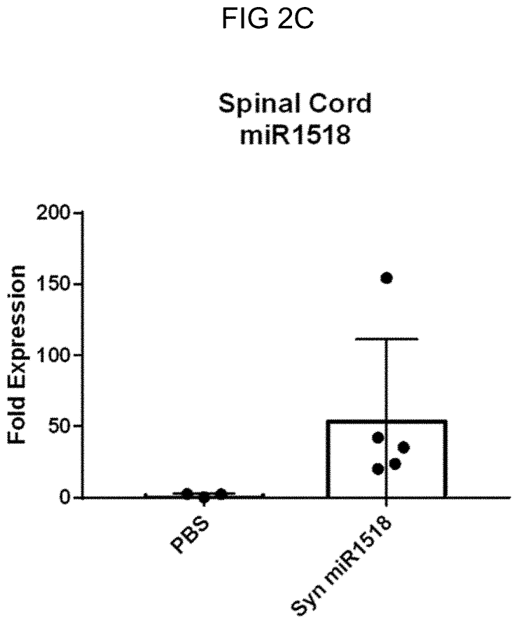

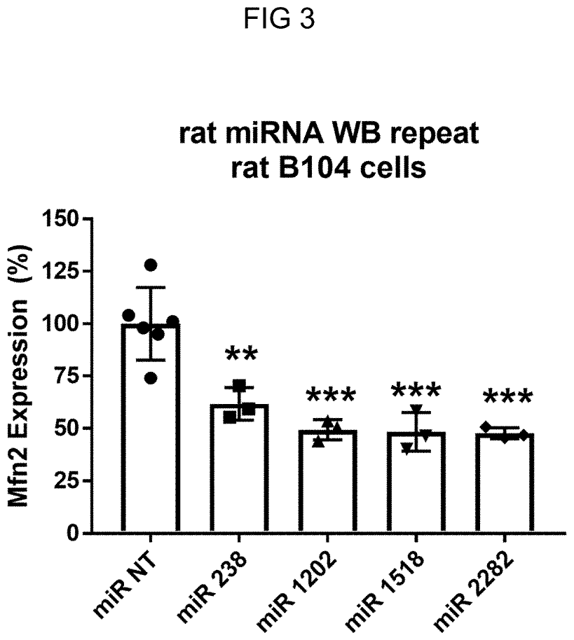

FIG. 2A to FIG. 2C illustrate Mfn2 RNA fold expression following delivery AAV vector comprising rat Mfn2 (rMfn2) cDNA transgene and mR1518, wherein AAV vector was administered in mice intravenously at a dose of 3×10¹¹ GC. Mice were necropsied at day 14 post administration, following which brain and spinal cord tissues were harvested and homogenized. RNA was extracted from the samples and used for qPCR with TaqMan primers against Mfn2 Two treatment groups were examined (N=5 per group): PBS and AAV9.PHP.eB.CB7.miR_1518+rMfn2.

### Example 2: Mouse Study of AAV Vector Comprising Expression Cassette of an Engineered Mfn2 Transgene with miRNA

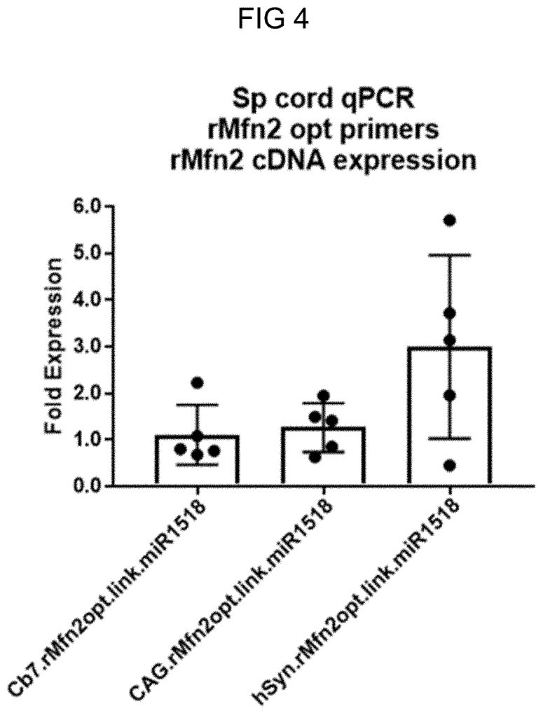

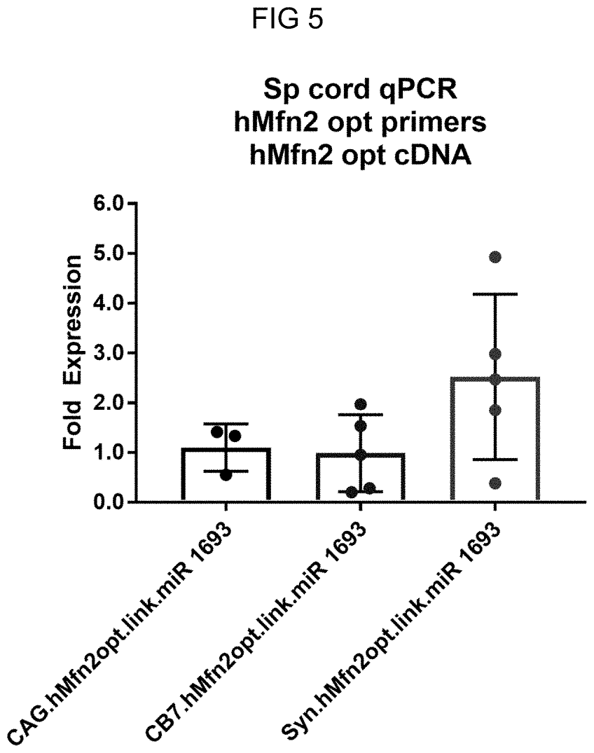

In initial study, wildtype mice were injected intravenously with 3×10¹¹ GC of AAV vectors, comprising an engineered rat Mfn2 (rMfn2) cDNA nucleic acid sequence (SEQ ID NO: 12) and miRNA1518 (SEQ ID NO: 16). Four treatment groups were examined (N=5 in each group): PBS, AAV9-PHP.eB.CAG rMfn2 opt.link.rMfn2miR1518.WPRE.SV40, AAV9-PHP.eB.CB7.CI.rMfn2 opt.link.rMfn2miR1518.rBG, and AAV9-PHP.eB.hSyn.PI.rMfn2opt.link.rMfn2miR1518.WPRE.bGH (wherein the vector genome is of SEQ ID NO: 3). Mice were sacrificed 14 days post injection. Brain and spinal cord were harvested and homogenized. RNA was extracted from the samples and used for qPCR with TaqMan primers against rat Mfn2 engineered cDNA. FIG. 4 illustrates plotted quantitation of fold-expression of rat Mfn2 (rMfn2) cDNA expression in spinal cord of treated mice following AAV vector delivery of engineered rMfn2 cDNA transgene with miR1518.

In a further examination, wildtype mice were injected intravenously with 3×10¹¹ GC of AAV vectors, comprising an engineered human Mfn2 (hMfn2) cDNA nucleic acid sequence (SEQ ID NO: 11) and miRNA1693 (SEQ ID NO: 15). Four treatment groups were examined (N=5 in each group): PBS, AAV9-PHP.eB.CAG.hMfn2opt.link.hMfn2miR1693.WPRE.SV40, AAV9-PHP.eB.CB7.CI.hMfn2opt.link.hMfn2miR1693.rBG, AAV9-PHP.eB.hSyn.PI.hMfn2opt.link.hMfn2miR1693.WPRE.SV40 (wherein vector genome is of SEQ ID NO: 1). Mice were sacrificed 14 days post injection. Brain and spinal cord were harvested and homogenized. RNA was extracted from the samples and used for qPCR with TaqMan primers against human Mfn2 engineered cDNA. FIG. 5 illustrates plotted quantitation of fold-expression of human Mfn2 (hMfn2) cDNA expression in spinal cord of treated mice following AAV vector delivery of engineered hMfn2 cDNA transgene with miR1693.

In both studied examining human and rat Mfn2 expression, among AAV vectors delivered, various promoters were examined, such as CB7, CAG and neuron specific human synapsin (hSyn). AAV vectors comprising hSyn promoters exhibited highest fold-expression of Mfn2 cDNA in spinal cord tissue.

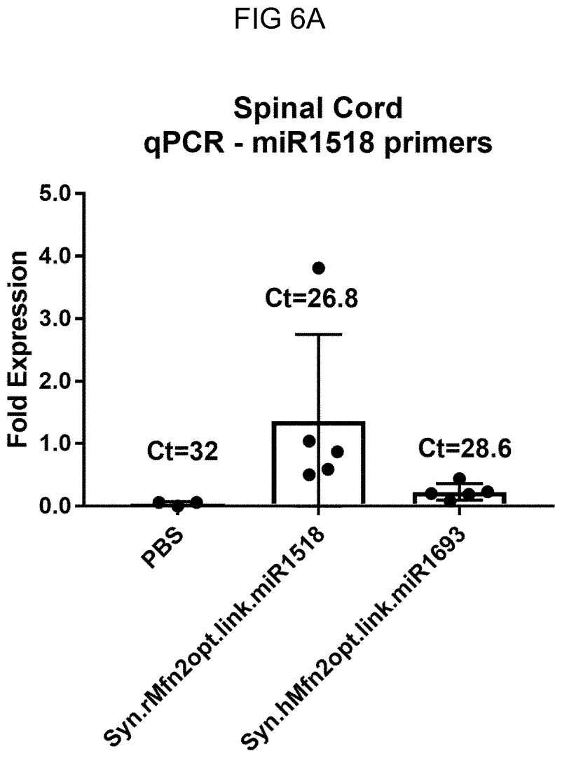

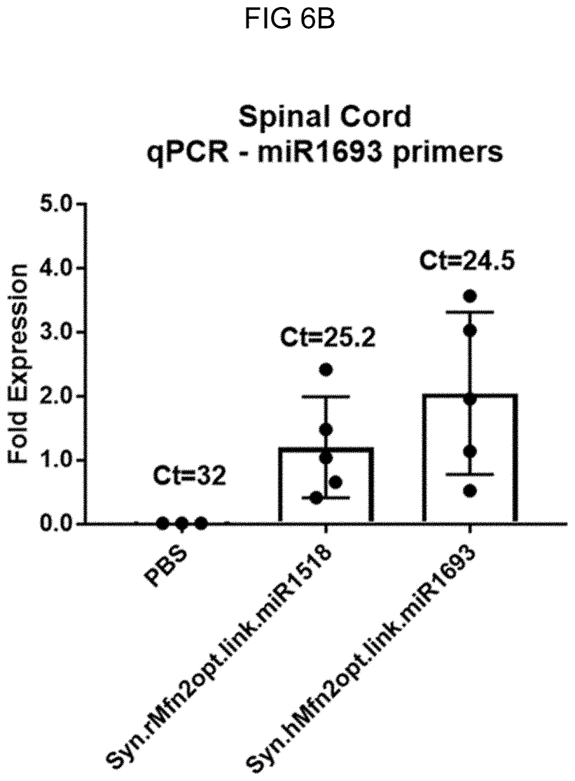

Furthermore, total amount of mature miRNA processed from AAV vectors were examined using an established procedure disclosed in Chen et al., Real-time quantification of micro-RNAs by stem-loop RT-PCR, Nucleic Acid Research, 2005, 33 (20). Wildtype mice were injected intravenously with 3×10¹¹ GC of AAV vectors or control group PBS. AAV genome vector of treatment group 1 comprised expression cassette for hMfn2 with miR1693 (SEQ ID NO: 1) which further comprising an engineered human Mfn2 (hMfn2) cDNA nucleic acid sequence (SEQ ID NO: 11) connected via linker (SEQ ID NO: 17) to miRNA1693 (SEQ ID NO: 15). AAV vector of treatment group 2 comprising an expression cassette for rMfn2 with miR1518 (SEQ ID NO: 3), further comprising an engineered rat Mfn2 (rMfn2) cDNA nucleic acid sequence (SEQ ID NO: 12) connected via linker (SEQ ID NO: 17) to miRNA1518 (SEQ ID NO: 16). Following treatment, mice were sacrificed at 14 days post injection. Brain and spinal cord were harvested and homogenized. RNA was extracted from the samples and used for custom miRNA Assay. The custom miRNA assay consists of a custom stem-loop RT primer to create the cDNA and then custom small RNA:stem loop TaqMan primers to amplify the miRNA. This assay measures the total amount of mature miRNA processed from the vector. FIG. 6A and FIG. 6B illustrate total amount of mature miRNA processed from the AAV vectors following intravenous delivery in mice.

In summary, hSyn promoter (SEQ ID NO: 6) appears to be best promoter for maximum spinal cord expression. AAV vectors comprising “link” (for “linker”) is an important spacer between the cDNA and miRNA necessary for proper miRNA excision and processing for RNAi. A smaller linker was tried due to cloning ease but was not functional. qPCR analysis suggests adequate expression of rat Mfn2 and human Mfn2 when co-delivered with miRNA targeting endogenous mutant Mfn2.

### Example 3. Transfection Efficiency of AAV Vectors in HEK293 and Mfn2-Null Cells

In this study we generated and examined various AAV vectors comprising a transgene of human Mfn2 engineered sequence, as listed in Table 3 below.

TABLE 3

| | SEQ ID NOs |
|---|---|
| AAV Vector | (AAV vector) |
| CB7.CI.hMfn2.GA.RBG | 79 |
| CB7.CI.hMfn2.GA.LINK.miR1518.RBG | 77 |
| CB7.CI.hMfn2.GA.LINK.miR538.RBG | 75 |
| CAG.CI.hMfn2.GA.WPRE.SV40 | 73 |
| CAG.CI.hMfn2.GA.LINK.miR1518.WPRE.SV40 | 71 |
| CAG.CI.hMfn2.GA.LINK.miR538.WPRE.SV40 | 69 |

Mfn2 Null MEF is a mouse cell line lacking Mfn2 which was used to detect expression of Mfn2 cDNA in vectors. Additionally, HEK293 cells, which is a human cell line which express Mfn 2, was used in transfection with above-identified vectors. Following transfection cell lysates were analyzed for overall Mfn protein expression via western blot, endogenous Mfn2 knockdown via qPCR, and presence of miRNA via qPCR. Western blot quantification was performed with Wes platform. The analysis of Mfn expression in Mfn2-null MEF cells quantified only Mfn2 protein produced by vector because cell line lacks Mfn2 expression. For qPCR miRNA detection assay, a stem-loop primer was used for reverse transcription (RT) of mature miRNA, a TaqMan probe set was used for amplification of mature miRNA to show expression and/or processing of miRNA. For qPCR for measuring endogenous human Mfn2 knockdown, a TaqMan primer/probe set was used, which distinguishes between HEK293 endogenous Mfn2 and vector Mfn2 For Wes-based Mfn2 protein quantitation and antibody to Mfn2 was used which cross-reacts with endogenous and vector produced Mfn2, therefore probing for overall Mfn2 overexpression.

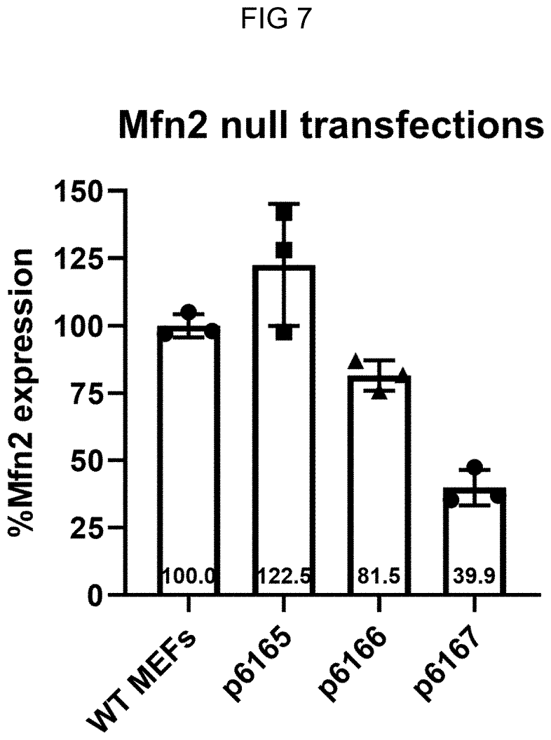

FIG. 7 shows expression levels of Mfn2 in Mfn2-null MEF cell line following transfection with various vectors comprising CB7 promoter. Furthermore, FIG. 7 shows plotted quantitation of western blot signal measuring expression of mitofurin-2 (Mfn2) following transfections with CB7.CI.hMfn2 GA.WPRE.RBG (p6165); CB7.CI.hMfn2.GA.LINK.miR1518.RBG (p6166); CB7.CI.hMfn2.GA.LINK.miR538.RBG (p6167). Quantitation is plotted as percent expression; transfection efficiency was determined to be about 40% as measured by flow cytometry and approximated by visual observation. For a western blot probed for expression levels of Mfn2 (data not shown), b-actin was used as a loading control (Mfn2: 2 μg loaded; b-actin: 0.27 μg loaded; exposure used for quantification 4 seconds).

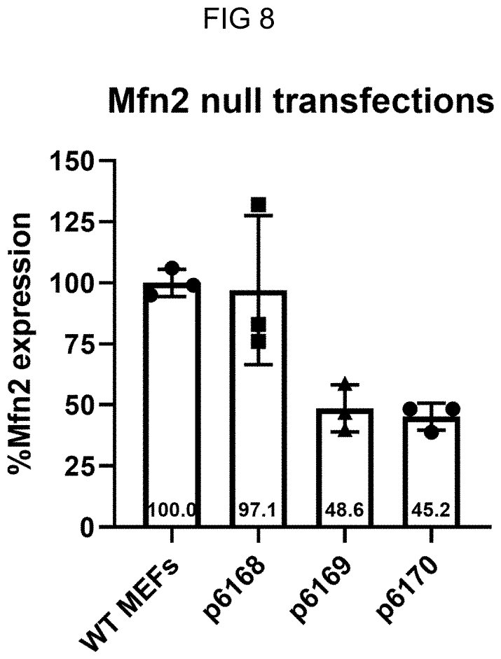

FIG. 8 shows expression levels of Mfn2 in Mfn2-null MEF cell line following transfection with various vectors comprising CAG promoter. Furthermore, FIG. 8 shows plotted quantitation of western blot signal measuring expression of mitofurin-2 (Mfn2) following transfections with CAG.CI.hMfn2 GA.WPRE.SV40 (p6168); CAG.CI.hMfn2 GA.LINK.miR1518.WPRE.SV40 (p6169); CAG.CI.hMfn2.GA.LINK.miR538.WPRE.SV40 (p6170). Quantitation is plotted as percent expression; transfection efficiency was determined to be about 40% as measured by flow cytometry and approximated by visual observation. For a western blot probed for expression levels of Mfn2, b-actin was used as a loading control (Mfn2: 2 μg loaded; b-actin: 0.27 loaded; exposure used for quantification 4 seconds).

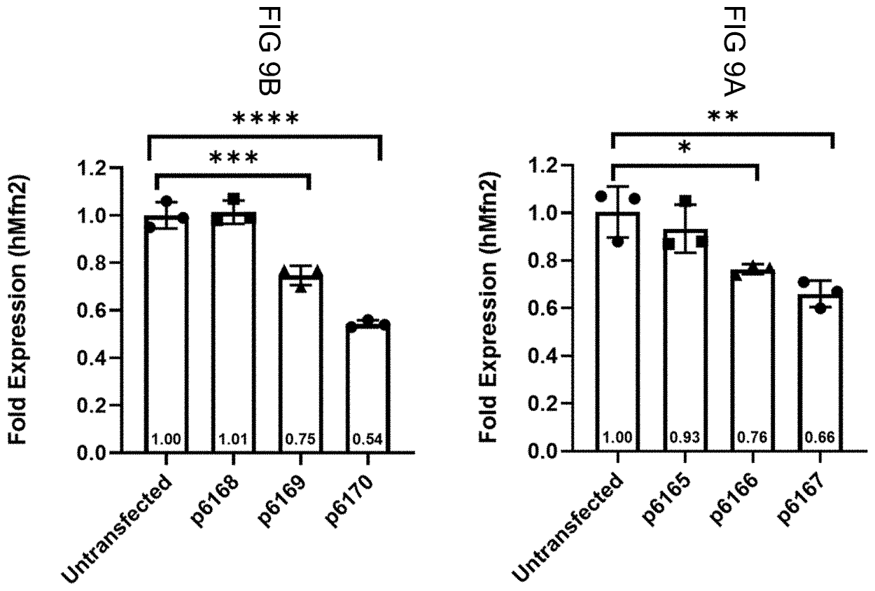

FIG. 9A and FIG. 9B show expression levels of Mfn2 in HEK293 cell line following transfection with various vectors comprising either CB7 or CAG promoter. High transfection efficiency was observed. FIG. 9A show endogenous Mfn2 knockdown in HEK293 cells as measured by qPCR and plotted as fold expression, following transfection with various vectors comprising CB7 promoter, (CB7.CI.hMfn2.GA.WPRE.RBG (p6165); CB7.CI.hMfn2.GA.LINK.miR1518.RBG (p6166); CB7.CI.hMfn2.GA.LINK.miR538.RBG(p6167)). FIG. 9B show endogenous Mfn2 knockdown in HEK293 cells as measured by qPCR and plotted as fold expression, following transfection with various vectors comprising CAG promoter (CAG.CI.hMfn2 GA.WPRE.SV40 (p6168); CAG.CI.hMfn2 GA.LINK.miR1518.WPRE.SV40 (p6169); CAG.CI.hMfn2 GA.LINK.miR538.WPRE.SV40 (p6170)).

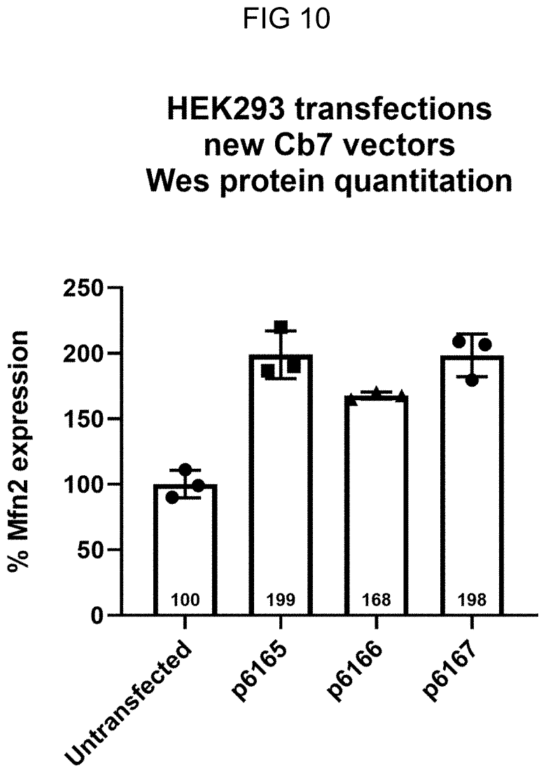

FIG. 10 shows expression levels of Mfn2 (endogenous Mfn2 and Mfn2 expressed from vector) in HEK293 cell line following transfection with various vectors comprising CB7 promoter. Furthermore, FIG. 10 shows plotted quantitation of western blot signal measuring expression of mitofurin-2 (Mfn2) following transfections with CB7.CI.hMfn2.GA.WPRE.RBG (p6165); CB7.CI.hMfn2.GA.LINK.miR1518.RBG (p6166); CB7.CI.hMfn2.GA.LINK.miR538.RBG (p6167). Quantitation is plotted as percent expression; transfection efficiency was determined to be about 95%. For a western blot probed for expression levels of Mfn2, b-actin was used as a loading control (Mfn2: 0.78 μg loaded; b-actin: 0.78 μg loaded; exposure used for quantification 4 seconds).

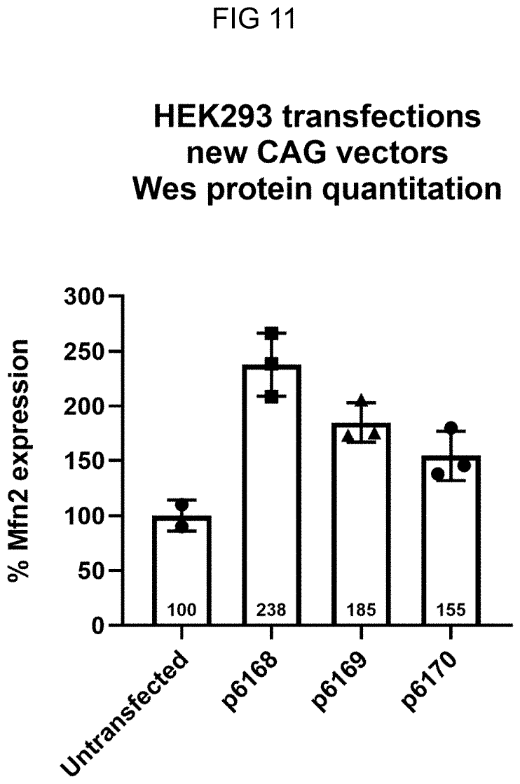

FIG. 11 shows expression levels of Mfn2 (endogenous Mfn2 and Mfn2 expressed from vector) in HEK293 cell line following transfection with various vectors comprising CAG promoter. Furthermore, FIG. 11 shows plotted quantitation of western blot signal measuring expression of mitofurin-2 (Mfn2) following transfections with CAG.CI.hMfn2 GA.WPRE.SV40 (p6168); CAG.CI.hMfn2.GA.LINK.miR1518.WPRE.SV40 (p6169); CAG.CI.hMfn2.GA.LINK.miR538.WPRE.SV40 (p6170). Quantitation is plotted as percent expression; transfection efficiency was determined to be about 95%. For a western blot probed for expression levels of Mfn2, b-actin was used as a loading control (Mfn2: 0.78 μg loaded; b-actin: 0.78 μg loaded; exposure used for quantification 4 seconds).

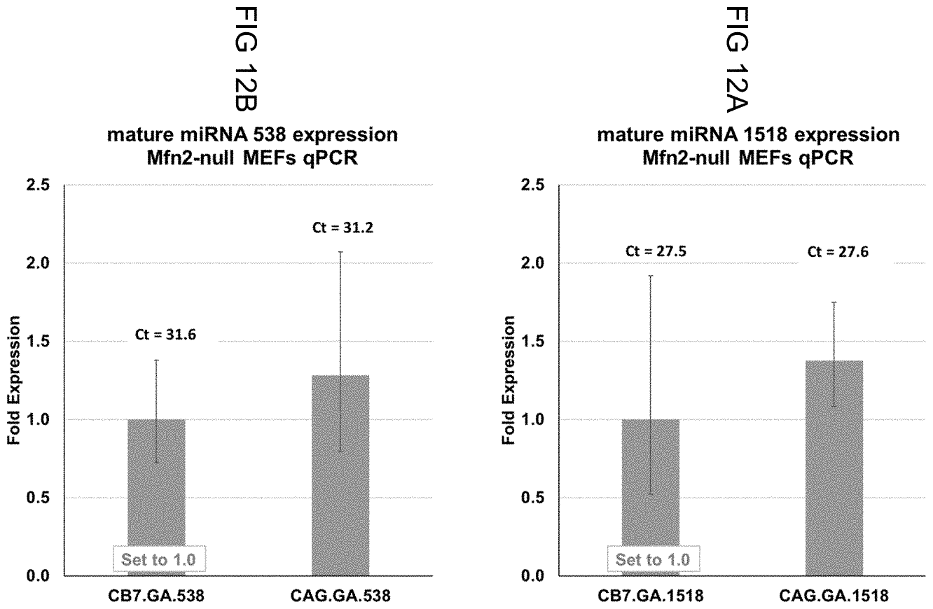

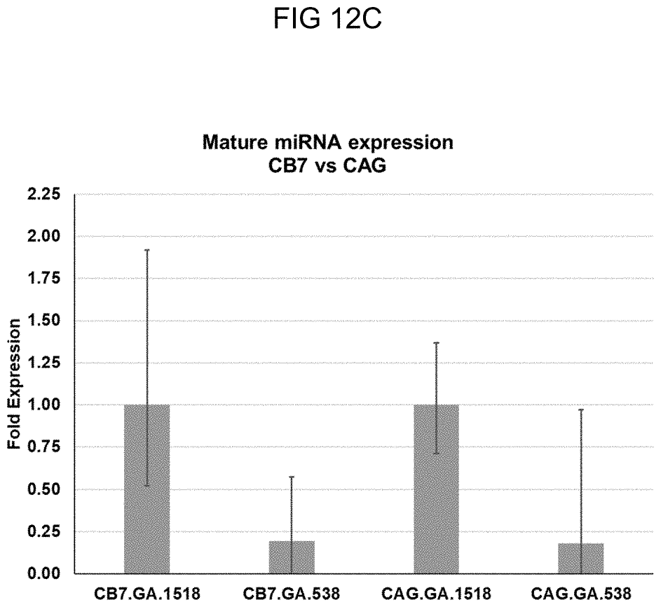

FIG. 12A to FIG. 12C show expression levels, as measured by qPCR, of mature miRNA (miR1518 or MiR538) in Mfn2-null MEF cell line, following transfection with various vectors comprising either CB7 or CAG promoter. FIG. 12A shows a comparison of expression levels, as measured by qPCR and plotted as fold expression, of mature miR1518 in Mfn2-null MEF cell line, following transfection with vectors comprising either CB7 or CAG promoter. FIG. 12B shows a comparison of expression levels, as measured by qPCR and plotted as fold expression, of mature miR538 in Mfn2-null MEF cell line, following transfection with vectors comprising either CB7 or CAG promoter. FIG. 12C shows a comparison of expression levels, as measured by qPCR and plotted as fold expression, of mature miR1518 and miR538 in Mfn2-null MEF cell line, following transfection with vectors comprising either CB7 or CAG promoter.

### Example 4. Gene Therapy Vectors for Efficacy in MFN2^{R94Q} Mice (C57BL/6J-Tg(Thy1-MFN2*), a Model of Charcot-Marie-Tooth Disease Type 2A

Seven (7) hemizygous male MFN2^{R94Q} mice (C57BL/6J-Tg(Thy1-MFN2*)44Balo/J, JAX stock #029745), two (2) male C57BL/6J mice (JAX stock #000664), and eighteen (18) female C57BL/6J mice (JAX stock #000664) were transferred to our in vivo research laboratory in Bar Harbor, Me. The mice were ear notched for identification, genotype(s) confirmed and housed in individually and positively ventilated polysulfonate cages with HEPA filtered air at a density of 3 mice per cage (two females with one male). The animal room wad lighted entirely with artificial fluorescent lighting, with a controlled 12 h light/dark cycle (6 am to 6 pm light). The normal temperature and relative humidity ranges in the animal rooms were 22±4° C. and 50±15%, respectively. The animal rooms were set to have 15 air exchanges per hour. Filtered tap water, acidified to a pH of 2.5 to 3.0, and normal rodent chow were provided ad libitum. The mice were used as breeders to raise the study cohort of 10 male C57BL/6J mice and 60 male hemizygous MFN2^{R94Q} mice in two rounds of breeding. At P0-P1, a total of seventy (70) mice were enrolled in the study.

TABLE 4

| | # | | | Age at | Dosing | Dosing | Tissue |
|---|---|---|---|---|---|---|---|
| Group | Mice | Genotype | Compound | Dosing | Route | Frequency | Collection |
| 1 | 10 | WT | Vehicle | P0-P1 | ICV | Single | Spinal |
| 2 | 10 | HEMI | Vehicle | | injection | Dose | cord; |
| 3 | 10 | HEMI | Vector 1 | | | | Tibial |
| 4 | 10 | HEMI | Vector 2 | | | | nerve |
| 5 | 10 | HEMI | Vector 3 | | | | |
| 6 | 10 | HEMI | Vector 4 | | | | |
| 7 | 10 | HEMI | Vector 5 | | | | |
| Total | 70 | | | | | | |

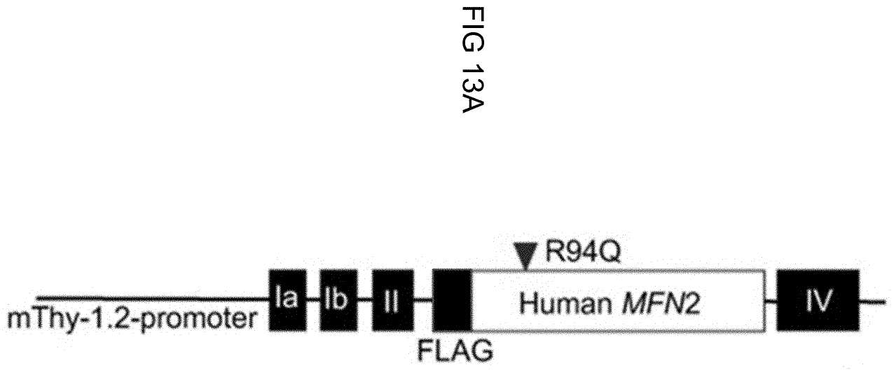

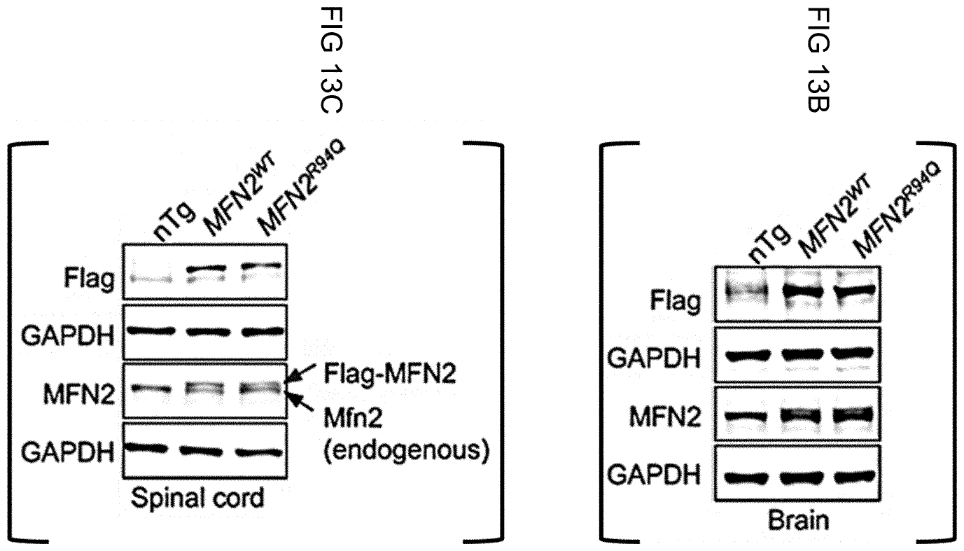

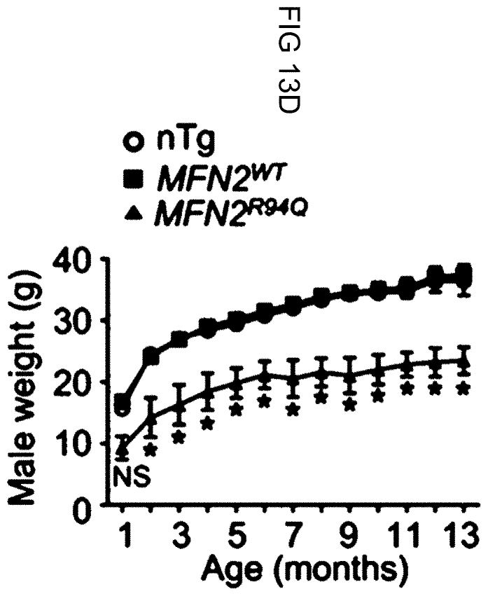

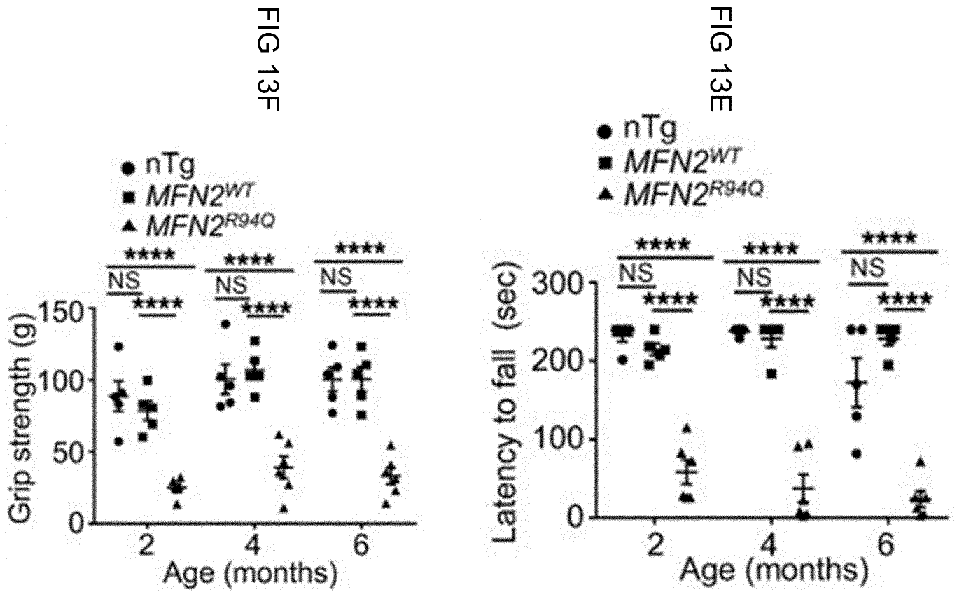

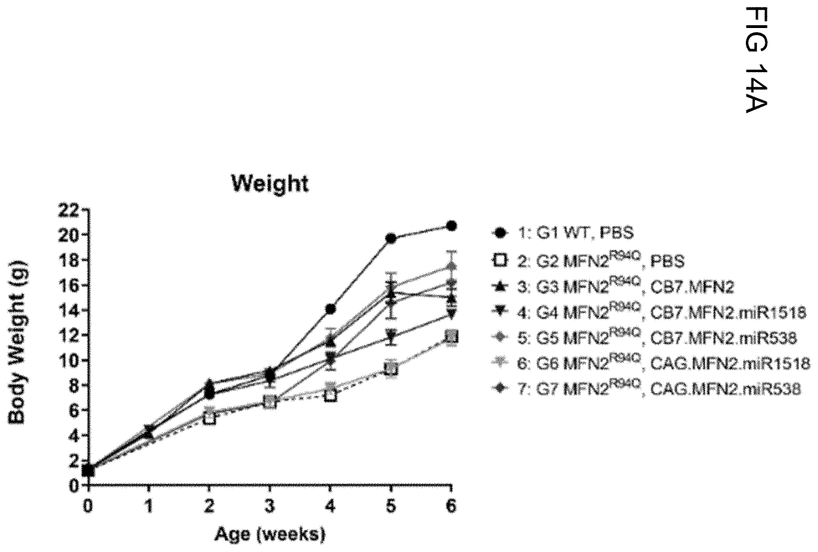

FIG. 13A to FIG. 13F show characterization of mouse model. FIG. 13A show schematic representation of the mice genotype. FIG. 13B shows mice phenotype characterization, characterized by relative expression levels endogenous and FLAG-tagged MFN2 in brain as measured by western blotting. FIG. 13C shows mice phenotype characterization, characterized by relative expression levels endogenous and FLAG-tagged MFN2 in spial cord as measured by western blotting. FIG. 13D shows measured weight in (g) of the mice in CMT2A mouse model (nTg, MFN2^{WT}, and MFN2^{R49Q}). FIG. 13E shows mice phenotype characterization, as measured by the latency to fall (sec). FIG. 13F shows mice phenotype characterization, as measured by grip strength (g).

In this study, mice are dosed at P0-P1 by neonatal ICV injection according to the table 4 above. Body weights of mice are measured weekly. At 15-17 weeks of age, the following tests are performed on each mouse: rotarod test, grip strength test, visual acuity test (optokinetic response), optional procedure. Additionally, Compound Muscle Action Potential (CMAP) testing is performed. At 17-19 weeks of age, mice are necropsied and the following tissues are collected: spinal cord, tibial nerve. The collected tissues are fixed in EM fixative and embedded in epoxy resin. One section is cut from each tissue. Sections are the stained with Toluidine Blue. Stained slides are scanned. For each of the scanned sections, the following parameters are determined: axon size distributions, axon counts, axon areas. The axon area distributions are then plotted for each group.

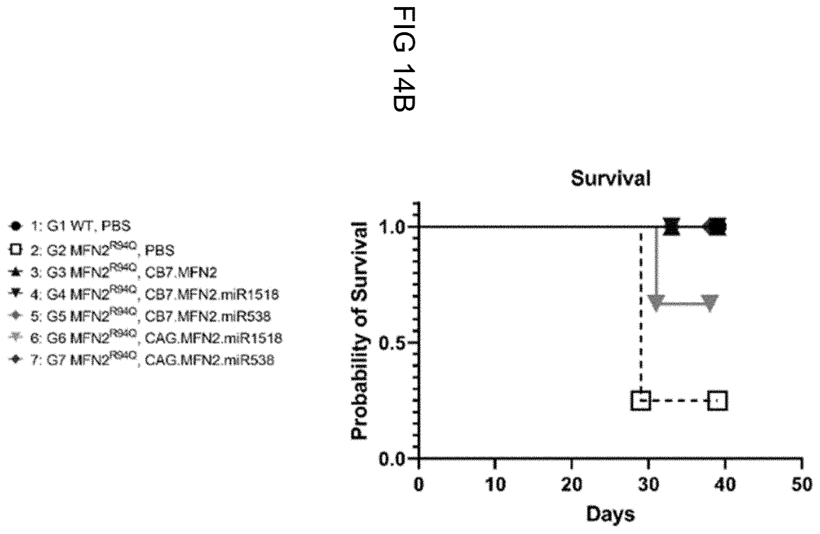

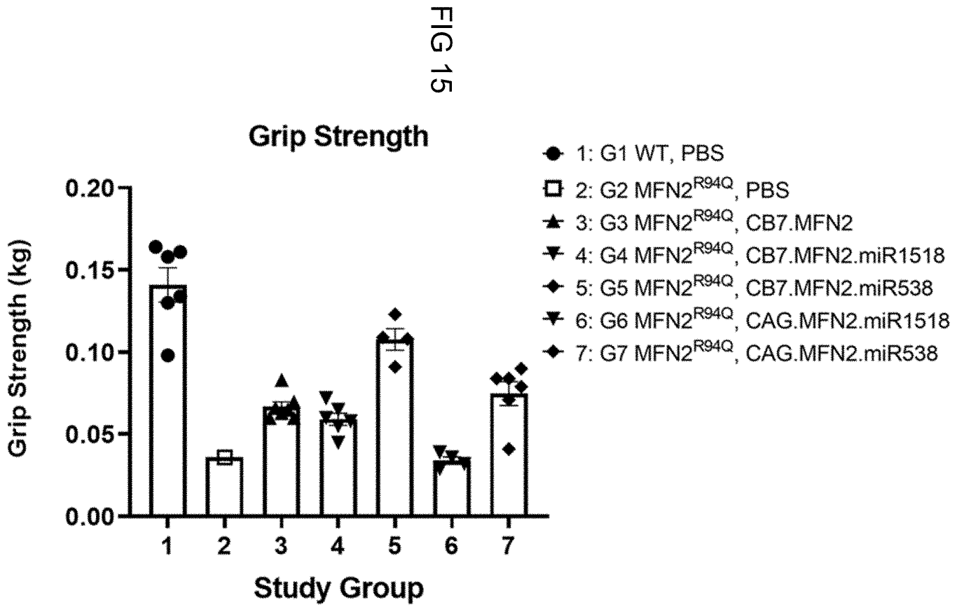

In a pharmacological study, for the first cohort, 4-7 mice per group were used. The study was a blinded study. Briefly, male MFN2″ mice were used in the treatment groups to which candidate AAVhu68 vectors were administered. Newborn ICV injection was used as a route of administration, with bilaterally administered dose of 3 μL of 7.5×10¹⁰ GC AAV vectors. Weight was measured weekly. Grip strength was measured at 6 weeks. FIG. 14A and FIG. 14B show results of the pilot pharmacological study in MFN2^{R94Q} mice (Study groups: G1—wild type (WT) mice, PBS; G2—MFN2^{R94Q} mice, PBS; G3—MFN2^{R94Q} mice, CB7.MFN2; G4—MFN2^{R94Q} mice, CB7.MFN2.miR1518; G5—MFN2^{R94Q} mice, CB7.MFN2.miR538; G6—MFN2^{R94Q} mice, CAG.MFN2.miR1518; G7—MFN2^{R94Q} mice, CAG.MFN2.miR538). FIG. 14A shows body weight results (plotted as (g)) as measured in mice groups G1 to G7. FIG. 14B shows survival results (plotted as probability of survival over day 0 to 50) as measured in mice groups G1 to G7. FIG. 15 shows grip strength results (plotted as (kg)) of the pilot pharmacological study in MFN2^{R94Q} mice.

### Example 5. AAV Vector Co-Administering hMfn2 and miR Targeting Endogenous hMfn2

In this study, AAVhu68.CB7.CI.hMfn2.miR538.RBG (also referenced as AAVhu68.CB7.CI.hMfn2.GA.LINK.miR538.RBG) vector is administered in Rhesus Macaque (Macaca mulatta) or Cynomolgus Macaque (non-human primates (NHP) via Intra-Cisterna Magna (ICM) injection, utilizing cerebrospinal fluid (CSF) to achieve widespread distribution from a single injection.

Test Article. The AAVhu68.CB7.CI.hMfn2.GA.LINK.miR538.RBG is used in this study. A certificate of analysis verifying quality and purity of the test article will be included in the final study report.

Preparation. Calculations for test article dilutions are performed and verified by trained GTP personnel prior to making dilutions. Test article dilutions are performed by designated personnel on the day of injection. Test article dilutions are verified by additional designated personnel. Diluted test article are kept on wet ice or at 2-8° C. for up to 8 hours until injection.

Archival Samples. Archival samples of test and control articles are retained by designated personnel and stored at ≤−60° C.

Test Article Analysis. The designated personnel is responsible for assuring that the test article meets the release requirements. A certificate of analysis is provided by Vector Core for inclusion with study records. All results are recorded. Copies of the data are provided to for inclusion in the study notebook.

Unused Test Article. Unused test article provided to NPRP personnel are stored on wet ice prior to being returned for archiving. Archival samples are stored at ≤−60° C.

Test System. Rhesus Macaque (Macaca mulatta) or Cynomolgus Macaque is used in this study.

Justification for Test System Selection. This study involves intra-cisterna magna (ICM) delivery of a miRNA test article (vector) for CNS diseases. The dimensions of the CNS in the NHP act as a representative model of our clinical target population. This study provides critical data on the dose and route of administration-related pharmacokinetics and safety of the test article after ICM injection in rhesus macaques. In this study, 2 animals are used. The NHPs are selected from male or female, 4-5 years of age, and about 3-10 kg.

Acclimation Period and Care. Quarantine and acclimation, animal husbandry and care are conducted in accordance with Standard Operating Procedures (SOP) procedures. Macaques are housed in stainless steel caging in CTRB. Husbandry and care are provided by DVR personnel.

Certified Primate Diet 5048 or a similar diet appropriate for nonhuman primates is supplied to study animals. Water is available from an automatic watering system and is accessible to all macaques ad libitum. Macaques are monitored by the veterinary staff for any conditions requiring possible intervention. The Study Director is consulted whenever possible to determine the appropriate course of action. However, in emergency situations, including possible euthanasia, decisions are made by the veterinary staff as needed and the Study Director is advised as soon as possible. Enrichment in the form of food rewards, conspecific interaction, and manipulanda are provided. Animals are maintained on a 12-hour light/dark cycle controlled by Building Automation. The desired temperature range is 64-84° F. (18-29° C.). Temperature is maintained in this range to the maximum extent possible. The desired humidity range is 30-70%. Humidity is maintained in this range to the maximum extent possible. Each macaque has been previously identified with a unique identification number that has been tattooed on its chest by the supplier. Both animals are included in a single treatment group.

Study design procedures. Two rhesus macaques are used in the study. Both macaques are included in a single treatment group. Baseline blood samples, including CSF, CBC, serum chemistry, and baseline biomarker assessment are collected from all macaques on day 0 prior to dosing. Vitals are also obtained from each macaque.

On day 0, macaques are sedated; weighed and vital signs are recorded prior to test article administration.

For test Article Administration (AAVhu68.CB7.CI.hMfn2.GA.LINK.miR538.RBG), macaques are anesthetized and appropriate analgesics are administered in accordance with SOP. Macaques are dosed with test article via sub-occipital puncture into the cisterna magna. For the Intra-Cisterna Magna (ICM) Puncture Procedure, anesthetized macaques are transferred from animal holding to the Procedure Room and placed on an x-ray table in the lateral decubitus position with the head flexed forward for CSF collection and dosing into the cisterna magna. The site of injection is aseptically prepared. Using aseptic technique, a 21-27 gauge, 1-1.5 inch Quincke spinal needle (Becton Dickinson) is advanced into the sub-occipital space until the flow of CSF is observed. 1 mL of CSF is collected for baseline analysis, prior to dosing. The anatomical structures that is traversed include the skin, subcutaneous fat, epidural space, dura and atlanto-occipital fascia. The needle is directed at the wider superior gap of the cisterna magna to avoid blood contamination and potential brainstem injury. Correct placement of needle puncture is verified via myelography, using a fluoroscope (OEC9800 C-Arm, GE). After CSF collection, a small bore T extension catheter is connected to the spinal needle to facilitate dosing of Iohexol (Trade Name: Omnipaque 180 mg/mL, General Electric Healthcare) contrast media and test article. Up to 2 mL of Iohexol is administered via the catheter and spinal needle. After verifying needle placement via CSF return and visualization of the needle by fluoroscopy, a syringe containing the test article is connected to a flexible linker containing the test article and injected at a rate of approximately 2 ml/minute. After administration, the needle is removed, and direct pressure is applied to the puncture site. The macaques receive ICM administration of test article (AAVhu68.CB7.CI.hMfn2.GA.LINK.miR538.RBG) at a dose of 3×10¹³ GC (3.33×10¹¹ GC/g brain). The total volume of injected per macaque is 1.0 mL. Doses and volumes are documented in the study records.

For observations, on selected time points as listed on the Study Schedule (Table 5), the macaque(s) are monitored for additional parameters beyond the daily observations, including but not limited to vital signs, clinical pathology, collection of serum and CSF.

For blood draws, animals are bled from a peripheral vein for a general safety panel which includes: neutralizing antibodies to AAV, hematology, clinical chemistry. Procedures for blood collection are performed in accordance with SOP. Additional blood samples are collected to measure changes in biomarkers (pharmacodynamic markers). The frequency of blood draws for complete blood counts, serum chemistry, biomarkers, neutralizing antibodies to AAV are as defined in the Study Schedule (Table 5) of the study protocol.

For antibody assays (neutralizing antibodies to AAV, serum), the blood sample (up to 2 mL) is collected via red top tubes (w/ or w/o serum separator), allowed to clot, and centrifuged. Study personnel isolates the serum. Serum is divided into two individually marked microcentrifuge tubes (labeled with Study number, Animal ID, Group number, time point, neutralizing Abs, and date) for every Antibody time point and stored at ≤−60° C.

For cell counts and differentials, blood samples for complete blood counts with differentials and platelet count are collected in labeled lavender top tubes (Study number, Animal ID, Group number, time point, CBC and date of collection), up to 2 mL and stored at 4° C. Blood is sent to Antech Diagnostics, Inc. overnight (with ice packs for lavender top tubes) for blood cell counts including platelet counts and differentials. The following parameters are analyzed for red cell count, hemoglobin, hematocrit, platelet count, leukocyte count, leukocyte differential, mean corpuscular volume (MCV), mean corpuscular hemoglobin (MCH), red blood cell morphology.

For clinical chemistries, blood samples of (up to 2 mL) are collected in labeled red top tubes, allowed to clot, and centrifuged. The serum is separated, put into labeled microcentrifuge tubes (Study number, Animal ID, Group number, time point, Chemistry and date of collection, and sent to Antech Diagnostics, Inc. overnight on ice packs for analysis. The following parameters are analyzed: alkaline phosphatase, bilirubin (total), creatinine, gamma-Glutamyl Transpeptidase, glucose, serum Alanine Aminotransferase, serum aspartate aminotransferase, albumin, albumin/globulin ratio (calculated), aldolase, blood urea nitrogen, calcium, chloride, creatinine kinase and isoforms, globulin (calculated as total protein minus albumin=globulin), lactate dehydrogenase, phosphorus, inorganic, potassium, sodium, total protein.

For biomarker analysis, blood sample (up to 2 mL) is collected via red top tubes (w/ or w/o serum separator), allowed to clot, and centrifuged. Study personnel isolates the serum. Serum is then divided into 2—individually marked microcentrifuge tubes (Study number, Animal ID, Group number, time point, serum biomarker, and date of collection). Samples are be stored at ≤−60° C.

For PBMC/tissue lymphocyte isolation and ELISPOT, blood samples (6-10 mL) are collected into sodium heparin (green top tubes) and PBMCs are isolated according to SOP. Blood collection tubes are labeled with Study number, Animal ID, Group number, Species, Time point, PBMC, and Date of Collection. Lymphocytes are harvested from spleen, liver and bone marrow according to SOPs. ELISPOT for capsid and transgene T cell responses are performed according to SOP.

For CSF collection, up to 1 mL of CSF is collected for analysis and biomarker assessment. For all CSF collection time points including Study Day 0, macaques are anesthetized in accordance with SOP and transferred from animal holding to the Procedure Room where they are placed in the lateral decubitus position with the head flexed forward for CSF collection. The hair over the back of the head and cervical spine is clipped. The occipital protuberance at the back of the skull and the wings of the atlas (C1) is palpated. The site of injection is aseptically prepared. Using aseptic technique, a 21-27 gauge, 1-1.5 inch Quincke spinal needle (BD) or 21-27 gauge needle is advanced into the cisterna magna until the flow of CSF is observed. The anatomical structures that are traversed include the skin, subcutaneous fat, epidural space, dura and atlanto-occipital fascia. The needle is directed at the wider superior gap of the cisterna magna to avoid blood contamination and potential brainstem injury. Post CSF collection, the needle is removed and direct pressure applied to the puncture site. Samples are collected in sterile 1.5 mL Eppendorf tubes labeled with the Study number, Animal ID, group number, time point, CSF, and date of collection. Samples are placed on wet ice, and immediately aliquoted for CSF clin pathology (blood cell counts and differentials and total protein quantification) and CSF biomarkers. CSF is collected at frequencies outlined in the Study Schedule (Table 5) of the study protocol.

For CSF clin pathology, CSF (0.5 mL) is aliquoted into lavender top tubes (labeled with the Study number, Animal ID, group number, time point, CSF clin path, and date of with ice packs) for blood cell counts and differentials and total protein quantification.

For CSF biomarkers, all remaining CSF (unused for CSF din path—up to 1 mL) collected in 2 sterile 1.5 mL Eppendorf tubes is centrifuged at 800 g for 5 minutes. Study personnel isolates the supernatant and aliquot into cryovials (labeled with the Study number, Animal ID, group number, time point, CSF biomarker, and date of collection). Samples are shipped on wet ice.

Additionally, on all days selected for monitoring, each macaque is sedated, weighed and have its respiratory and heart rates monitored, and body temperature taken via rectal thermometers prior to any blood samples being taken. The blood samples for PBMC isolation are transported at room temperature. Other samples are transported on wet ice.

The study is performed according to the schedule as described in Table 5 below.

TABLE 5

| Study | | |
|---|---|---|
| Timepoint | Sample/Procedure | N |
| Study Day | Body Weight, Temperature, Respiratory | 2 |
| 0 | Rate, Heart Rate | |
| | Biomarker (baseline) (RC) | 2 |
| | CSF (baseline) (CV + LT) | 2 |
| | Neurological Monitoring | 2 |
| | Clin path (baseline) (LT, RC) | 2 |
| | Nerve conduction velocity testing | 2 |
| | Antibodies (baseline) (RC) | 2 |
| | Immunology (baseline) (PBMC) (GT) | 2 |
| | ICM vector 3 × 10¹³ GC | 2 |
| Study Day | Body Weight, Temperature, Respiratory | 2 |
| 7 | Rate, Heart Rate | |
| (±2 days) | Biomarker (RC) | 2 |
| | Clin path (LT, RC) | 2 |
| Study Day | Body Weight, Temperature, Respiratory | 2 |
| 14 | Rate, Heart Rate | |
| (±2 days) | Biomarker (RC) | 2 |
| | CSF (CV + LT) | 2 |
| | Clin path (LT, RC) | 2 |
| Study Day | Body Weight, Temperature, Respiratory | 2 |
| 21 | Rate, Heart Rate | |
| (±2 days) | Biomarker (RC) | 2 |
| | Clin path (LT, RC) | 2 |
| Study Day | Body Weight, Temperature, Respiratory | 2 |
| 28 | Rate, Heart Rate | |
| (±2 days) | | |
| | Biomarker (RC) | 2 |
| | Clin path (LT, RC) | 2 |
| | Neurological Monitoring | 2 |
| | Nerve conduction velocity testing | 2 |
| Terminal | Body Weight, Temperature, Respiratory | 2 |
| Day 35 | Rate, Heart Rate | |
| (±2 days) | Neurological monitoring | 2 |
| | Biomarker (RC) | 2 |
| | CSF (CV + LT) | 2 |
| | Clin path (LT, RC) | 2 |
| | Antibodies (RC) | 2 |
| | Immunology (PBMC) (GT) | 2 |
| | Nerve conduction velocity testing | 2 |
| | Necropsy | 2 |
| | Tissues to be harvested | 2 |

When macaques are necropsied, tissues described in Table 6 below are collected.

TABLE 6
Tissues to be Collected at Time of Necropsy for Histopathology and Biodistribution

| | Histopathology | | Biodistribution | | Lymphocyte |
|---|---|---|---|---|---|
| Organ | Req. | Coll. | Req. | Coll. | isolation |
| Chest tattoo | ✓ | | | | |
| Ascending aorta (proximal) | ✓ | | ✓ | | |
| Bone marrow, rib | ✓ | | | | |
| Bone marrow, femur | | | ✓ | | ✓ |
| Brain right | ✓ | | | | |
| hemisphere^{A} | | | | | |
| Frontal Cortex^{A} | | | ✓ | | |
| Parietal Cortex^{A} | | | ✓ | | |
| Temporal Cortex^{A} | | | ✓ | | |
| Occipital Cortex^{A} | | | ✓ | | |
| Hippocampus^{A} | | | ✓ | | |
| Cerebellum^{A} | | | ✓ | | |
| Medulla^{A} | | | ✓ | | |
| Cranial nerves IX, X, XI | ✓ R&L | | | | |
| Dorsal root | ✓ | | ✓ | | |
| ganglia, cervical^{B} | | | | | |
| Dorsal root | ✓ | | ✓ | | |
| ganglia, thoracic^{B} | | | | | |
| Dorsal root | ✓ | | ✓ | | |
| ganglia, lumbar^{B} | | | | | |
| Epididymis | ✓ right | | ✓ left | | |
| Eye | ✓ right | | ✓ left | | |
| Gall bladder | ✓ | | ✓ | | |
| Heart^{C} | ✓ | | ✓ left | | |
| | | | ventricle | | |
| Kidney, right | ✓ | | ✓ | | |
| Kidney, left | ✓ | | ✓ | | |
| Liver, left lobe | ✓ | | ✓ | | ✓ |
| Liver, right lobe | ✓ | | ✓ | | |
| Lung, left | ✓ | | ✓ | | |
| Lung, right | ✓ | | ✓ | | |
| Lymph node, deep cervical | | | ✓ | | |
| Lymph node, mandibular | ✓ | | ✓ | | |
| Lymph node, mesenteric | ✓ | | ✓ | | |
| Muscle, quadriceps femoris | ✓ right | | ✓ left | | |
| Nerve, sciatic | ✓ R&L | | ✓ left | | |
| Nerve, tibial | ✓ R&L | | ✓ left | | |
| Nerve, peroneal | ✓ R&L | | ✓ left | | |
| Nerve, median Proximal | ✓ R&L | | ✓ left | | |
| Nerve, median Distal | ✓ R&L | | | | |
| Nerve, radial | ✓ R&L | | ✓ left | | |
| Nerve, sural | ✓ R&L | | ✓ left | | |
| Nerve, ulnar | ✓ R&L | | ✓ left | | |
| Optic nerve (CN II) | ✓ right | | ✓ left | | |
| Ovary | ✓ right | | ✓ left | | |
| Pancreas | ✓ | | ✓ | | |
| Pituitary | ✓ | | ✓ | | |
| Skin with mammary | ✓ | | | | |
| Injection site | ✓ | | | | |
| Spinal cord, cervical^{D} | ✓ | | ✓ | | |
| Spinal cord, thoracic^{D} | ✓ | | ✓ | | |
| Spinal cord, lumbar^{D} | ✓ | | ✓ | | |
| Spleen | ✓ | | ✓ | | ✓ |
| Testis | ✓ right | | ✓ left | | |
| Thymus | ✓ | | ✓ | | |
| Thyroid gland (with parathyroid) | ✓ right | | ✓ left | | |
| Trigeminal nerve ganglia | ✓ right | | ✓ left^{E} | | |
| Gross lesions (if any)^{F} | ✓ | | ✓ | | |

✓ = Tissue will be collected and processed for indicated assay, Req. = Required by Protocol, Coll. = Collected, N/A = Not Applicable
Abbreviations: L, left; R, right
^{A}The brain is collected whole and then divided in half along the midsagittal plane. The entire right half of the brain is placed in 10% neutral buffered formalin for histopathology and further trimmed and processed according to “Pardo et al., 2012, Technical Guide for Nervous System Sampling of the Cynomolgus Monkey for General Toxicity Studies, Toxicologic Pathology 40: 624-636”. Samples for biodistribution are collected from the left hemisphere and stored at ≤−60° C.
^{B}A minimum of 3 cervical, 3 thoracic and 3 lumbar dorsal root ganglia are sampled and fixed in three tissue cassettes for histopathology (cervical, thoracic, lumbar). In addition, the 3 contralateral cervical dorsal root ganglia, 3 contralateral thoracic dorsal root ganglia, and 3 contralateral 3 lumbar dorsal root ganglia are sampled and frozen ≤−60° C. for biodistribution.
^{C}Sections of heart should include the right and left ventricles (with AV valves) and interventricular septum (with valves).
^{D}Each spinal cord is divided into cervical, thoracic, and lumbar segments labeled C, T, or L, respectively. Each spinal cord segment is divided into three sections. The 3 C, T, or L sections will each be numbered 1-3. From each animal, there is a total of 9 spinal cord sections generated, numbered C1-3, T1-3, and L1-3. From each spinal cord segment, section 1 (C1, T1, L1) is used for histopathological analysis. From each spinal cord segment, section 2 (C2, T2, L2) are used for biodistribution analysis (RNA and protein analysis, 2 tubes). From each spinal cord segment, section 3 (C3, T3, L3) are formalin fixed and paraffin embedded for LCM.
^{E}Portion of trigeminal nerve is collected for possible occupational health exposure and stored in ≤−60° C.
^{F}Gross observations made at necropsy for which histopathology is not appropriate (e.g. fluid, matted hair, missing anatomic parts) are collected.

### Experimental Evaluation: Observations

For viability checks (In-Cage), the macaques are monitored by daily visual observation for general appearance, signs of toxicity which may include but are not limited to neurologic signs or lethargy, distress and changes in behavior. This is performed by personnel in accordance with SOPs. The Veterinarian and Study Director are notified for any unusual conditions. Treatment are conducted only after approval by the Veterinarian and Study Director, except in case of emergency imperiling the macaque or for humanely sacrificing the macaque if the Secondary Veterinarian and Study Director cannot be contacted in a timely manner.

Macaques found dead or euthanized due to their moribund state are evaluated in the same manner as terminal sacrificed macaques. A complete set of tissues will be collected and any gross lesions recorded.

Additionally, all macaques are visually examined each time they are anesthetized. At the time of necropsy, the macaques are examined for gross abnormalities. All changes are noted. Macaques are weighed at the beginning of the study, at necropsy, and at every time point in which they are sedated.

Food consumption is not be monitored. Water is administered ad lib and standard diet with enrichment foods are given to the macaques.

On the days selected for monitoring, Sensory Nerve Conduction Studies are performed.

For neurological exam, on the days selected for monitoring, a Nonhuman Primate Neurological Assessment is performed. The neurological assessment is divided in to 5 sections: Mentation, Posture and Gait, Proprioception, Cranial Nerves, and Spinal Reflexes.

For postmortem analysis, gross postmortem examinations are performed on all macaques, including macaques euthanized in a moribund condition or found dead. All abnormal observations are recorded.

Since this study requires an ongoing evaluation of animals, therefore time of necropsy is determined at a later time point. Any animals that die unexpectedly are necropsied as soon as practical. For all unscheduled deaths, clinical pathology, extensive gross pathology, and histopathology are performed on a complete list of tissues, which are collected under the discretion of the study pathologist. Other analyses (immune response, etc.) are included as appropriate in an attempt to determine possible cause of death. Tissues are collected on all animals as outlined in table 6. Gross observations made at necropsy for which histopathology is not appropriate (e.g., fluid, matted hair, missing anatomic parts) are not collected.

The macaques surviving to the end of their study period are euthanized. The macaques are first be sedated and are then euthanized Death is confirmed by absence of heartbeat and respiration. The macaque may be exsanguinated to help ensure death.

All tissues are preserved which are listed in Table 6. Tissues are fixed by placement in 10% neutral buffered formalin for paraffin embedding. Eyes used for histopathologic examination are fixed in modified Davidson's solution prior to paraffin embedding.

For biodistribution analysis, a section of each tissue in table 6 is frozen down to −80° C. as quickly as possible. At the discretion of the Study Director, DNA is extracted from tissues, and vector biodistribution is evaluated by quantitative PCR.

For histopathological analysis, a subset or all tissues in Table 6 are stained with hematoxylin and eosin (H&E). Additional procedures and/or other stains may be employed at the discretion of the Study Pathologist to identify/clarify histologic features and are documented in the final report. All data documenting experimental details and study procedures and observations are recorded and maintained in the study binder. At the completion of the study, all reports and raw data are maintained in the archives. Preserved specimens and tissues are archived and stored. Study Director determines the need for final disposition of these materials. All data documenting experimental details and study procedures and observations are recorded and maintained by the GTP personnel in dedicated notebooks and study binders.

Assays which are Performed on Tissues Collected at Necropsy

Following conclusion of the study, macaques are necropsied with tissues harvested for full pathology (for list of tissues collected at necropsy see Table 6).

For histopathology, hematoxylin and eosin (H&E) stains are performed on all or a subset of tissues listed for collection for histopathology, at the discretion of the Study Director. Additional appropriate stains may be employed based on histopathological findings. For Mfn2 cDNA and miRNA expression, tissue sections preserved in formalin and paraffin embedded (FFPE) are processed and motor neurons are laser-capture microdissected. Motor neurons isolated from the spinal cord are RNA extracted and qPCR is performed to determine level of Mfn2 knockdown, levels of Mfn2 cDNA expression and levels of miRNA expression using specific primer sets. Knockdown of Mfn2 and expression of Mfn2 cDNA may also be evaluated in tissue sample lysates by qPCR and Western blot Immunohistochemistry for Mfn2 may be performed on brain and spinal cord tissues. Briefly, paraffin sections are incubated with an antibody raised against the Mfn2 protein. For biodistribution, a section of a section of each tissue in Table 6 is frozen down to ≤−60° C. as quickly as possible. DNA may be extracted from tissues, and vector biodistribution is evaluated by quantitative PCR. For tissue lymphocyte isolation and ELISPOT, lymphocytes may be harvested from spleen and bone marrow and ELISPOT for capsid and transgene T cell responses are performed.

### Sequence Listing Free Text

The following information is provided for sequences containing free text under numeric identifier <223>.

| SEQ ID NO: | Free Text under <223> |
|---|---|
| 1 | <223> Vector Genome |
| | Syn.PI.hMfn2opt.link.hMfn2 miR1693.WPRE.bGH |
| | <220> |
| | <221> repeat_region |
| | <222> (1)..(141) |
| | <223> 5′ITR |
| | <220> |
| | <221> promoter |
| | <222> (223)..(682) |
| | <223> hSynapsin |
| | <220> |
| | <221> Intron |
| | <222> (694)..(826) |
| | <223> Promega Chimeric Intron |
| | <220> |
| | <221> misc_signal |
| | <222> (857)..(862) |
| | <223> Kozak |
| | <220> |
| | <221> CDS |
| | <222> (863)..(3136) |
| | <223> human Mfn2 engineered |
| | <220> |
| | <221> misc_feature |
| | <222> (3134)..(3139) |
| | <223> Dbl stop codon |
| | <220> |
| | <221> misc_feature |
| | <222> (3140)..(3229) |
| | <223> linker |
| | <220> |
| | <221> misc_binding |
| | <222> (3230)..(3252) |
| | <223> 5′flank |
| | <220> |
| | <221> misc_feature |
| | <222> (3253)..(3316) |
| | <223> hMfn2 miR1693 |
| | <220> |
| | <221> misc_binding |
| | <222> (3317)..(3360) |
| | <223> 3′flank |
| | <220> |
| | <221> misc_feature |
| | <222> (3463)..(4045) |
| | <223> WRPE |
| | <220> |
| | <221> misc_feature |
| | <222> (4410)..(4455) |
| | <223> Additional AAV squences |
| | <220> |
| | <221> repeat_region |
| | <222> (4456)..(4585) |
| | <223> 3′ITR |
| 2 | <223> Synthetic Construct |
| 3 | <223> 5′ITR |
| | <220> |
| | <221> promoter |
| | <222> (223)..(682) |
| | <223> hSynapsin |
| | <220> |
| | <221> Intron |
| | <222> (694)..(826) |
| | <223> Promega Chimeric Intron |
| | <220> |
| | <221> CDS |
| | <222> (863)..(3133) |
| | <223> rMfn2 engineered |
| | <220> |
| | <221> misc_feature |
| | <222> (3134)..(3139) |
| | <223> Dbl stop codon |
| | <220> |
| | <221> misc_feature |
| | <222> (3140)..(3229) |
| | <223> linker |
| | <220> |
| | <221> misc_signal |
| | <222> (3230)..(3252) |
| | <223> 5′flank |
| | <220> |
| | <221> misc_feature |
| | <222> (3253)..(3316) |
| | <223> rMfn2 miR1518 |
| | <220> |
| | <221> misc_signal |
| | <222> (3317)..(3360) |
| | <223> 3′flank |
| | <220> |
| | <221> enhancer |
| | <222> (3457)..(4044) |
| | <223> WRPE |
| | <220> |
| | <221> misc_feature |
| | <222> (4385)..(4430) |
| | <223> Additional AAV sequences |
| | <220> |
| | <221> repeat_region |
| | <222> (4431)..(4560) |
| | <223> 3′ITR |
| 4 | <223> Synthetic Construct |
| 5 | <223> 5′ITR |
| 6 | <223> hSynapsin promoter |
| 7 | <223> Promega Chimeric Intron |
| 8 | <223> WPRE |
| 9 | <223> additonal AAV sequences |
| 10 | <223> 3′ ITR |
| 11 | <223> human Mfn2 engineered nucleic acid sequence |
| | encoding mitofusin 2 protein |
| 12 | <223> rMfn2 engineered nucleic acid sequence |
| | encoding mitofusin 2 protein |
| 13 | <223> 5′flank |
| 14 | <223> 3′flank |
| 15 | <223> hMfn2 miR1693 |
| 16 | <223> rMfn2 miR1518 |
| 17 | <223> linker |
| 18 | <223> hMfn2 endogenous nucleic acid sequence |
| 19 | <223> Synthetic Construct |
| 20 | <223> rMfn2 endogenous nucleic acid sequence |
| 21 | <223> Synthetic Construct |
| 22 | <223> bGH poly A |
| 23 | <223> SV40 polyA |
| 24 | <223> hMfn2 engineered nucleic acid sequnece V1 |
| 25-46 | <223> Synthetic Construct |
| 47-67 | <223> Target hMfn2 Sequence |
| 68 | <223> miR1693(21-mer) |
| 69 | <223> Vector genome |
| | CAG.CI.hMfn2.GA.link.miR538.WPRE.SV40 |
| | <220> |
| | <221> repeat_region |
| | <222> (1)..(188) |
| | <223> 5′ITR |
| | <220> |
| | <221> misc_feature |
| | <222> (192)..(1032) |
| | <223> CAG(CBA) |
| | <220> |
| | <221> enhancer |
| | <222> (194)..(436) |
| | <223> CMV enhancer |
| | <220> |
| | <221> promoter |
| | <222> (439)..(720) |
| | <223> CB promoter |
| | <220> |
| | <221> Intron |
| | <222> (720)..(1127) |
| | <223> Hybrid Intron in CAG |
| | <220> |
| | <221> Intron |
| | <222> (720)..(950) |
| | <223> b-actin intron |
| | <220> |
| | <221> misc_feature |
| | <222> (1158)..(1163) |
| | <223> Kozak |
| | <220> |
| | <221> CDS |
| | <222> (1164)..(3437) |
| | <223> human Mfn2 engineered |
| | <220> |
| | <221> misc_feature |
| | <222> (1379)..(1399) |
| | <223> miR538 mut |
| | <220> |
| | <221> misc_feature |
| | <222> (2534)..(2554) |
| | <223> miR1518 mutated |
| | <220 |
| | <221> misc_feature |
| | <222> (3438)..(3519) |
| | <223> linker p4271 |
| | <220> |
| | <221> misc_feature |
| | <222> (3520)..(3547) |
| | <223> miR538 AS |
| | <220> |
| | <221> misc_feature |
| | <222> (3569)..(3587) |
| | <223> loop |
| | <220> |
| | <221> misc_feature |
| | <222> (3588)..(3606) |
| | <223> miR538 sense |
| | <220> |
| | <221> misc_feature |
| | <222> (3607)..(3651) |
| | <223> 3′ flank |
| | <220> |
| | <221> misc_feature |
| | <222> (3678)..(4266) |
| | <223> WRPE |
| | <220> |
| | <221> misc_feature |
| | <222> (4355)..(4474) |
| | <223> SV40 PA temrinator |
| | <220> |
| | <221> repeat_region |
| | <222> (4584)..(4771) |
| | <223> 3′ITR |
| 70 | <223> Synthetic Construct |
| 71 | <223> Vector genome |
| | CAG.CI.hMfn2.GA.link.miR1518.WPRE.SV40 |
| | <220> |
| | <221> repeat_region |
| | <222> (1)..(188) |
| | <223> ITR |
| | <220> |
| | <221> misc_feature |
| | <222> (192)..(1032) |
| | <223> CAG (CBA) |
| | <220> |
| | <221> enhancer |
| | <222> (194)..(436) |
| | <223> CMV enhancer |
| | <220> |
| | <221> promoter |
| | <222> (439)..(720) |
| | <223> CB Promoter |
| | <220> |
| | <221> Intron |
| | <222> (720)..(1127) |
| | <223> Hybrid intron in CAG |
| | <220> |
| | <221> Intron |
| | <222> (720)..(950) |
| | <223> b-actin intron |
| | <220> |
| | <221> misc_feature |
| | <222> (1158)..(1163) |
| | <223> Kozak |
| | <220> |
| | <221> CDS |
| | <222> (1164)..(3437) |
| | <223> human Mfn2 optimized |
| | <220> |
| | <221> misc_feature |
| | <222> (1379)..(1399) |
| | <223> miR538 mut |
| | <220> |
| | <221> misc_feature |
| | <222> (2534)..(2554) |
| | <223> miR 1518 mutated |
| | <220> |
| | <221> misc_feature |
| | <222> (3438)..(3519) |
| | <223> linker p4271 |
| | <220> |
| | <221> misc_feature |
| | <222> (3520)..(3547) |
| | <223> 5′ flank |
| | <220> |
| | <221> misc_feature |
| | <222> (3569)..(3587) |
| | <223> loop |
| | <220> |
| | <221> misc_feature |
| | <222> (3588)..(3606) |
| | <223> rat miR 1518 sense |
| | <220> |
| | <221> misc_feature |
| | <222> (3607)..(3651) |
| | <223> 3′ flank |
| | <220> |
| | <221> misc_feature |
| | <222> (3678)..(4266) |
| | <223> WRPE |
| | <220> |
| | <221> misc_feature |
| | <222> (4355)..(4474) |
| | <223> SV40 PA terminator |
| | <220> |
| | <221> repeat_region |
| | <222> (4584)..(4771) |
| | <223> ITR |
| | <220> |
| | <221> misc_feature |
| | <222> (35488)..(3568) |
| | <223> rat miR1518 AS |
| 72 | <223> Synthetic Construct |
| 73 | <223> Vector genome |
| | CAG.CI.hMfn2.GA.WPRE.SV40 |
| | <220> |
| | <221> repeat_region |
| | <222> (1)..(188) |
| | <223> ITR |
| | <220> |
| | <221> misc_feature |
| | <222> (192)..(1032) |
| | <223> CAG (CBA) |
| | <220> |
| | <221> enhancer |
| | <222> (194)..(1032) |
| | <223> CMV enhancer |
| | <220> |
| | <221> promoter |
| | <222> (439)..(720) |
| | <223> CB promoter |
| | <220> |
| | <221> Intron |
| | <222> (720)..(1127) |
| | <223> Hybrid intron in CAG |
| | <220> |
| | <221> Intron |
| | <222> (720)..(950) |
| | <223> b-actin intron |
| | <220> |
| | <221> misc_feature |
| | <222> (1158)..(1163) |
| | <223> Kozak |
| | <220> |
| | <221> CDS |
| | <222> (1164)..(3437) |
| | <223> huamn Mfn2 engineered |
| | <220> |
| | <221> misc_feature |
| | <222> (1379)..(1399) |
| | <223> miR538 mut |
| | <220> |
| | <221> misc_feature |
| | <222> (2534)..(2554) |
| | <223> miR1518 mutated |
| | <220> |
| | <221> misc_feature |
| | <222> (3466)..(4054) |
| | <223> WRPE |
| | <220> |
| | <221> misc_feature |
| | <222> (4143)..(4262) |
| | <223> SV 40 PA temrinator |
| | <220> |
| | <221> rep_origin |
| | <222> (4372)..(4559) |
| | <223> ITR |
| 74 | <223> Synthetic Construct |
| 75 | <223> Vector genome |
| | CB7.CI.hMfn2.GA.link.miR538.rBG |
| | <220> |
| | <221> repeat_region |
| | <222> (1)..(130) |
| | <223> ITR |
| | <220> |
| | <221> enhancer |
| | <222> (259)..(562) |
| | <223> CMV enhancer |
| | <220> |
| | <221> promoter |
| | <222> (561)..(842) |
| | <223> CB7 promoter |
| | <220> |
| | <221> Intron |
| | <222> (935)..(1907) |
| | <223> chicken beta-actin intron |
| | <220> |
| | <221> misc_feature |
| | <222> (1953)..(1958) |
| | <223> Kozak |
| | <220> |
| | <221> CDS |
| | <222> (1959)..(4232) |
| | <223> human Mfn2 engineered |
| | <220> |
| | <221> misc_feature |
| | <222> (2174)..(2194) |
| | <223> miR538 mut |
| | <220> |
| | <221> misc_feature |
| | <222> (3329)..(3349) |
| | <223> miR1518 |
| | <220> |
| | <221> misc_feature |
| | <222> (4233)..(4314) |
| | <223> linker p5271 |
| | <220> |
| | <221> misc_feature |
| | <222> (4315)..(4342) |
| | <223> 5′ flank |
| | <220> |
| | <221> misc_feature |
| | <222> (4343)..(4363) |
| | <223> miR538 AS |
| | <220> |
| | <221> misc_feature |
| | <222> (4364)..(4382) |
| | <223> loop |
| | <220> |
| | <221> misc_feature |
| | <222> (4383)..(4401) |
| | <223> miR538 Sense |
| | <220> |
| | <221> misc_feature |
| | <222> (4402)..(4446) |
| | <223> 3′ flank |
| | <220> |
| | <221> polyA_signal |
| | <222> (4500)..(4626) |
| | <223> rabbit globin poly A |
| | <220> |
| | <221> repeat_region |
| | <222> (4692)..(4821) |
| | <223> ITR |
| 76 | <223> Synthetic Construct |
| 77 | <223> Vector genome |
| | CB7.CI.hMfn2.GA.link.miR1518.rBG |
| | <220> |
| | <221> repeat_region |
| | <222> (1)..(130) |
| | <223> ITR |
| | <220> |
| | <221> enhancer |
| | <222> (259)..(562) |
| | <223> CMV enhancer |
| | <220> |
| | <221> promoter |
| | <222> (561)..(842) |
| | <223> CB7 promoter |
| | <220> |
| | <221> Intron |
| | <222> (935)..(1907) |
| | <223> chicken beta-actin intron |
| | <220> |
| | <221> misc_feature |
| | <222> (1953)..(1958) |
| | <223> Kozak |
| | <220> |
| | <221> CDS |
| | <222> (1959)..(4232) |
| | <223> human Mfn2 engineered |
| | <220> |
| | <221> misc_feature |
| | <222> (2174)..(2194) |
| | <223> miR538 mut |
| | <220> |
| | <221> misc_feature |
| | <222> (3329)..(3349) |
| | <223> miR 1518 mutated |
| | <220> |
| | <221> misc_feature |
| | <222> (4233)..(4314) |
| | <223> linker p4271 |
| | <220> |
| | <221> misc_feature |
| | <222> (4315)..(4342) |
| | <223> 5′ flank |
| | <220> |
| | <221> misc_feature |
| | <222> (4343)..(4363) |
| | <223> rat miR1518 AS |
| | <220> |
| | <221> misc_feature |
| | <222> (4364)..(4382) |
| | <223> loop |
| | <220> |
| | <221> misc_feature |
| | <222> (4383)..(4401) |
| | <223> rat miR 1518 sense |
| | <220> |
| | <221> misc_feature |
| | <222> (4402)..(4446) |
| | <223> 3′Flank |
| | <220> |
| | <221> polyA_signal |
| | <222> (4584)..(4710) |
| | <223> rabbit globin poly A |
| | <220> |
| | <221> repeat_region |
| | <222> (4776)..(4905) |
| | <223> ITR |
| 78 | <223> Synthetic Construct |
| 79 | <223> Vector genome CB7.CI.hMfn2.GA.rBG |
| | <220> |
| | <221> rep_origin |
| | <222> (1)..(130) |
| | <223> ITR |
| | <220> |
| | <221> enhancer |
| | <222> (259)..(562) |
| | <223> CMV enhancer |
| | <220> |
| | <221> promoter |
| | <222> (561)..(842) |
| | <223> CB7 promoter |
| | <220> |
| | <221> TATA_signal |
| | <222> (815)..(818) |
| | <223> TATA signal |
| | <220> |
| | <221> Intron |
| | <222> (935)..(1907) |
| | <223> Chicken beta-actin intron |
| | <220> |
| | <221> misc_feature |
| | <222> (1940)..(1948) |
| | <223> Kozak |
| | <220> |
| | <221> CDS |
| | <222> (1946)..(4219) |
| | <223> huamn Mfn2 engineered |
| | <220> |
| | <221> misc_feature |
| | <222> (2161)..(2181) |
| | <223> miR538 mut |
| | <220> |
| | <221> misc_feature |
| | <222> (3316)..(3336) |
| | <223> miR1518 mutated |
| | <220> |
| | <221> polyA_signal |
| | <222> (4244)..(4370) |
| | <223> rabbit globin poly A |
| | <220> |
| | <221> repeat_region |
| | <222> (4436)..(4565) |
| | <223> ITR |
| 80 | <223> Synthetic Construct |
| 81 | <223> AAVhu68 |
| 84 | <223> AAV-PHP.eB |
| 86 | <223> CB7 Promoter with CMV enhancer |
| 87 | <223> CAG promoter |
| 88 | <223> rabbit beta-globin |
| 89 | <223> miR1538 |
| 90 | <223> linker |

All patents, patent publications, and other publications listed in this specification, as well as the sequence listing file “20-9141PCT_SeqListing_ST25” are incorporated herein by reference. U.S. Provisional Patent Application No. 63/051,336, filed Jul. 13, 2020, and U.S. Provisional Patent Application No. 63/173,045 filed Apr. 9, 2021 are incorporated herein by reference. While the invention has been described with reference to a particularly preferred embodiment, it will be appreciated that modifications can be made without departing from the spirit of the invention. Such modifications are intended to fall within the scope of the appended claims.

1. A recombinant adeno-associated virus (rAAV) comprising an AAV capsid and packaged therein a vector genome, wherein the vector genome comprises:
(a) an engineered nucleic acid sequence encoding human mitofusin 2 (hMfn2);
(b) a spacer sequence located between (a) and (c);
(c) at least one miRNA coding sequence which is specific for a target site in an endogenous human mitofusin 2_nucleic acid sequence in a Charcot-Marie-Tooth (CMT2) patient;
wherein the engineered nucleic acid sequence of (a) lacks the target site for the at least one miRNA of (c), thereby preventing the at least one miRNA from targeting the engineered hMfn2 coding sequence; and
(d) regulatory sequences operably linked to (a) and (c) which direct expression thereof in a cell.

2. The rAAV according to claim 1, wherein the AAV capsid is selected from AAVrh91, AAV9, AAVhu68, or AAV1 capsid.

3. The rAAV according to claim 1, wherein the engineered hMfn2 coding sequence has a nucleic acid sequence of SEQ ID NO: 11 or a sequence at least 90% identical thereto.

4. The rAAV according to claim 1, wherein the engineered hMfn2 coding sequence has the nucleic acid sequence of SEQ ID NO: 11 or a sequence at least about 80% identical thereto, wherein nt 216 to 236 and/or nt 1371 to 1391 of the engineered hMfn2 coding sequence are conserved.

5. The rAAV according to claim 1, wherein the at least one miRNA comprises a sequence of one or more of an miRNA targeting sequence comprising SEQ ID NO: 89 (miR538, 59 nt) or a sequence at least 99% identical to SEQ ID NO: 89, or an miRNA comprising one or more of an antisense sequence of: AGAACAGGTTCTGGACGTCAA, SEQ ID NO: 27, wherein the at least one miRNA does not bind to the engineered hMfn2 coding sequence of (a) or its encoded messenger RNA (mRNA).

6. The rAAV according to claim 1, wherein the at least one miRNA coding sequence comprises a sequence of one or more of an miRNA targeting sequence comprising SEQ ID NO: 15 (miR1693, 64 nt), an miRNA comprising one or more of an antisense sequence of: AAACCTTGAGGACTACTGGAG, SEQ ID NO: 32, or an miRNA targeting sequence comprising SEQ ID NO: 16 (miR1518, 59 nt) or a sequence at least 99% identity to SEQ ID NO: 16, wherein the at least one miRNA does not bind to the engineered hMfn2 coding sequence of (a) or its encoded mRNA.

7. The rAAV according to claim 1, wherein the spacer is 75 nucleotides to about 250 nucleotides in length.

8. The rAAV according to claim 1, wherein the at least one miRNA coding sequence is 3′ to the engineered hMfn2 coding sequence.

9. The rAAV according to claim 1, wherein the at least one miRNA coding sequence is located within an intron sequence.

10. The rAAV according to claim 1, wherein the at least one miRNA coding sequence further comprising more than one miRNA coding sequence.

11. The rAAV according to claim 1, wherein the vector genome further comprises a constitutive promoter, optionally a CB7 promoter or a CAG promoter.

12. The rAAV according to claim 1, wherein the vector genome further comprises a tissue specific promoter which is a human synapsin (hSyn) promoter.

13. A vector comprising:
(a) an engineered nucleic acid sequence encoding human mitofusin 2 (hMfn2) operably linked to regulatory sequences which direct expression thereof in a human target cell; and/or
(b) a nucleic acid sequence encoding at least one hairpin forming miRNA which inhibits expression of endogenous human mitofusin 2 by specifically targeting a site on the mRNA of an endogenous human mitofusin 2 coding sequence which is not present in the engineered hMfn2 coding sequence, wherein the at least one hairpin miRNA coding sequence is operably linked to regulatory sequences which direct expression thereof in the human target cell, thereby preventing the at least one hairpin miRNA from reducing expression from the engineered hMfn2 coding sequence,
wherein the engineered hMfn2 coding sequence of (a) lacks a target site for the at least one hairpin miRNA encoded by (b).

14-19. (canceled)

20. The rAAV according to claim 1, wherein the at least one hairpin miRNA coding sequence comprises the sequence of one or more of:
(a) SEQ ID NO 15 (miR1693, 64 nt);
(b) at least 60 consecutive nucleotides of SEQ ID NO: 15;
(c) at least 99% identity to SEQ ID NO: 15 which comprises a sequence with 100% identity to about nucleotide 6 to about nucleotide 26 of SEQ ID NO: 15 (or SEQ ID NO: 68);
(d) a hairpin miRNA coding sequence which comprises a seed sequence of one or more of:
(i) TTGACGTCCAGAACCTGTTCT, SEQ ID NO: 27;
(ii) AGAAGTGGGCACTTAGAGTTG, SEQ ID NO: 28;
(iii) TTCAGAAGTGGGCACTTAGAG, SEQ ID NO: 29;
(iv) TTGTCAATCCAGCTGTCCAGC, SEQ ID NO: 30;
(v) CAAACTTGGTCTTCACTGCAG, SEQ ID NO: 31;
(vi) AAACCTTGAGGACTACTGGAG, SEQ ID NO: 32;
(vii) TAACCATGGAAACCATGAACT, SEQ ID NO: 33;
(viii) ACAACAAGAATGCCCATGGAG, SEQ ID NO: 34;
(ix) AAAGGTCCCAGACAGTTCCTG, SEQ ID NO: 35;
(x) TGTTCATGGCGGCAATTTCCT, SEQ ID NO: 36;
(xi) TGAGGTTGGCTATTGATTGAC, SEQ ID NO: 37;
(xii) TTCTCACACAGTCAACACCTT, SEQ ID NO: 38;
(xiii) TTTCCTCGCAGTAAACCTGCT, SEQ ID NO: 39;
(xiv) AGAAATGGAACTCAATGTCTT, SEQ ID NO: 40;
(xv) TGAACAGGACATCACCTGTGA, SEQ ID NO: 41;
(xvi) AATACAAGCAGGTATGTGAAC, SEQ ID NO: 42;
(xvii) TAAACCTGCTGCTCCCGAGCC, SEQ ID NO: 43;
(xviii) TAGAGGAGGCCATAGAGCCCA, SEQ ID NO: 44;
(xix) TCTACCCGCAGGAAGCAATTG, SEQ ID NO: 45; or
(xx) CTCCTTAGCAGACACAAAGAA, SEQ ID NO: 46, or combinations of any of (i) through (xx)
wherein the hairpin miRNA does not bind to the engineered hMfn2 coding sequence or its encoded mRNA.

21. The rAAV according to claim 1, comprising the engineered hMfn2 coding sequence and the at least one miRNA coding sequence with a single nucleic acid molecule and further comprising a spacer of at least 75 nucleotides between the engineered hMfn2 coding sequence and the at least one miRNA coding sequence.

22. The rAAV according to claim 1, wherein the miRNA coding sequence is 3′ to the engineered hMfn2 coding sequence.

23. The rAAV according to claim 1, wherein the miRNA coding sequence is located within an intron sequence.

24. The rAAV according to claim 1, further comprising more than one miRNA coding sequence.

25. The v rAAV according to claim 1, wherein the regulatory sequences comprise a constitutive promoter, optionally a CB7 promoter or CAG promoter.

26. The rAAV according to claim 1, wherein the regulatory sequences comprise a neuronal specific promoter, optionally a human synapsin promoter.

27. The vector according to claim 13, wherein the vector is a non-viral vector, or wherein the at least one non-viral vector is a liposome, or wherein the vector is a replication-incompetent, recombinant viral vector selected from a recombinant parvovirus, a recombinant lentivirus, or a recombinant herpes simplex virus.

28-37. (canceled)

38. A pharmaceutical composition comprising the rAAV according to claim 1, and a pharmaceutically acceptable aqueous suspending liquid, excipient, and/or diluent.

39. (canceled)

40. A method for treating a patient having Charcot-Marie-Tooth 2A (CMT2A) or for reducing neuropathy in a patient having Charcot-Marie-Tooth 2A (CMT2A) neuropathy comprising delivering an effective amount of the recombinant AAV according to claim 1.

41-43. (canceled)

44. A combination regimen for treating a patient having CMT2A which comprises co-administering
(a) a recombinant nucleic acid sequence comprising an engineered human mitofusin 2 (hMfn2) coding sequence, wherein the miRNA coding sequence comprises of SEQ ID NO: 89 or at least 99% identical to SEQ ID NO: 89, operably linked to regulatory sequences which direct expression thereof in a human target cell, wherein the engineered hMfn2 coding sequence has the sequence of SEQ ID NO: 11 or a sequence at least 95% identical thereto and which differs from an endogenous human mitofusin 2 coding sequence in the CMT2A patient by having a mismatch in an miRNA target site of (b),
(b) a nucleic acid comprising at least one coding sequence for an miRNA specific for the endogenous hMfn2 coding sequence in a human CMT2A subject, wherein the miRNA coding sequence is operably linked to regulatory sequences which direct expression thereof in the subject, wherein the at least one miRNA has a sequence of one or more of: an miRNA coding sequence comprising SEQ ID NO 15 (miR1693, 64 nt); an miRNA coding sequence comprising at least 60 consecutive nucleotides of SEQ ID NO: 15; an miRNA coding sequence comprising at least 99% identity to SEQ ID NO: 15 which comprises a sequence with 100% identity to about nucleotide 6 to about nucleotide 26 of SEQ ID NO: 15 (or SEQ ID NO: 68); or an miRNA coding sequence comprising one or more of:
(i) TTGACGTCCAGAACCTGTTCT, SEQ ID NO: 27;
(ii) AGAAGTGGGCACTTAGAGTTG, SEQ ID NO: 28;
(iii) TTCAGAAGTGGGCACTTAGAG, SEQ ID NO: 29;
(iv) TTGTCAATCCAGCTGTCCAGC, SEQ ID NO: 30;
(v) CAAACTTGGTCTTCACTGCAG, SEQ ID NO: 31;
(vi) AAACCTTGAGGACTACTGGAG, SEQ ID NO: 32;
(vii) TAACCATGGAAACCATGAACT, SEQ ID NO: 33;
(viii) ACAACAAGAATGCCCATGGAG, SEQ ID NO: 34;
(ix) AAAGGTCCCAGACAGTTCCTG, SEQ ID NO: 35;
(x) TGTTCATGGCGGCAATTTCCT, SEQ ID NO: 36;
(xi) TGAGGTTGGCTATTGATTGAC, SEQ ID NO: 37;
(xii) TTCTCACACAGTCAACACCTT, SEQ ID NO: 38;
(xiii) TTTCCTCGCAGTAAACCTGCT, SEQ ID NO: 39;
(xiv) AGAAATGGAACTCAATGTCTT, SEQ ID NO: 40;
(xiv) TGAACAGGACATCACCTGTGA, SEQ ID NO: 41;
(xvi) AATACAAGCAGGTATGTGAAC, SEQ ID NO: 42;
(xvii) TAAACCTGCTGCTCCCGAGCC, SEQ ID NO: 43;
(xviii) TAGAGGAGGCCATAGAGCCCA, SEQ ID NO: 44;
(xix) TCTACCCGCAGGAAGCAATTG, SEQ ID NO: 45; or
(xx) CTCCTTAGCAGACACAAAGAA, SEQ ID NO: 46, or
combinations of any of (i) through (xx),
(c) and wherein the miRNA does not bind to the engineered hMfn2 coding sequence of (a) or its encoded mRNA,
wherein a first vector is a viral vector or non-viral vector comprising the nucleic acid (a), wherein the first and the second viral vector may be from the same virus source or may be different, and a second, different vector, is a viral vector or non-viral vector comprising at least one miRNA (b), wherein the first and the second vectors may be same composition or may be different.

45-49. (canceled)


SEQUENCE LISTING (90 sequences)

SEQ ID NO: 1 - DNA, 4585 bp, Artificial Sequence
  Vector Genome Syn.PI.hMfn2opt.link.hMfn2 miR1693.WPRE.bGH
  repeat_region (1)..(141): 5′ITR
  promoter (223)..(682): hSynapsin
  Intron (694)..(826): Promega Chimeric Intron
  misc_signal (857)..(862): Kozak
  CDS (863)..(3136): human Mfn2 engineered
  misc_feature (3134)..(3139): Dbl stop codon
  misc_feature (3140)..(3229): linker
  misc_binding (3230)..(3252): 5′flank
  misc_feature (3253)..(3316): hMfn2 miR1693
  misc_binding (3317)..(3360): 3′flank
  misc_feature (3463)..(4045): WRPE
  misc_feature (4410)..(4455): Additional AAV squences
  repeat_region (4456)..(4585): 3′ITR
ccttaattaggctgcgcgctcgctcgctcactgaggccgggcgaccaaaggtcgcccgacgcccgggctttgcccgggcggcctcagtgagcgagcgagcgcgcagagagggagtggccaactccatcactaggggttccttgtagttaatgattaacccgccatgctacttatctacgtagccatgctctaggaagatcggaattcgcccttaagctagactcgagctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcggtaagtatcaaggttacaagacaggtttaaggagaccaatagaaactgggcttgtcgagacagagaagactcttgcgtttctgataggcacctattggtcttactgacatccactttgcctttctctccacagtacgtagaattcacgcgtgctagcaccggtgccaccatgagcctgctgttcagccggtgcaacagcatcgtgaccgtgaagaaaaacaagcggcacatggccgaagtgaacgcctctccactgaagcacttcgtgaccgccaagaagaagatcaacggcatcttcgagcagctgggcgcctacatccaagagagcgccaccttcctggaagatacctacagaaacgccgagctggaccccgtgaccacagaggaacaggtgctggatgtgaagggctacctgagcaaagtgcggggcatctctgaagtgctggccagacggcatatgaaggtggcctttttcggccggaccagcaacggcaagagcaccgtgatcaatgccatgctgtgggacaaagtgctgccctctggaatcggccacaccaccaactgctttctgagagtggaaggcaccgacggccacgaggcatttctgctgacagagggctccgaggaaaagcggagcgccaagacagtgaaccagctggcccatgctctgcaccaggataagcagctgcatgccggaagcctggtgtccgtgatgtggcccaatagcaagtgccctctgctgaaggacgacctggtgctgatggatagccccgggatcgatgtgaccaccgaactggacagctggatcgacaagttctgcctggacgccgacgtgttcgtgctggtggccaatagcgagagcaccctgatgcagaccgagaagcactttttccacaaggtgtccgagcggctgagcagacccaatatctttatcctgaacaacagatgggacgccagcgccagcgagcccgagtatatggaagaagtgcggcggcagcatatggaacggtgcacatcctttctggtggacgagctgggcgtcgtggatagatctcaggccggcgacagaatcttctttgtgtccgccaaagaggtgctgaacgcccggattcagaaagcccagggaatgcctgaaggcggaggtgcactggccgagggatttcaagtgcggatgttcgagttccagaacttcgagcggagattcgaggagtgcatcagccagagcgccgtcaagaccaagttcgagcagcataccgtgcgggccaagcagattgccgaagccgtcagactgatcatggacagcctgcatatggccgccagagagcagcaggtctactgcgaggaaatgcgggaagagagacaggaccggctgaagttcatcgacaagcagctggaactgctggcccaggactacaagctgcggatcaagcagatcaccgaagaggtggaaagacaggtgtccaccgctatggccgaggaaatcagacggctgagcgtgctggtcgacgactaccagatggactttcacccctctccagtggtgctgaaagtctacaaaaacgagctgcaccggcacatcgaggaaggcctgggcagaaacatgagcgacagatgcagcaccgccatcaccaatagcctgcagaccatgcagcaggacatgatcgacggcctgaaacctctgctgcctgtgtccgtcagatcccagatcgatatgctggtgccccggcagtgctttagcctgaactacgacctgaactgcgacaagctgtgcgccgatttccaagaggacatcgagtttcacttcagcctcggctggacaatgctggtcaacagatttctgggccccaagaacagcagacgggccctgatgggctacaacgatcaggtgcagaggcccattcctctgacacccgccaatcctagcatgcctccactgcctcagggcagcctgacacaagaggaattcatggtgtctatggtcaccggcctggccagcctgaccagcagaacatctatgggcatcctggtcgtcggcggcgttgtgtggaaagctgttggctggcggctgatcgccctgagctttggcctgtatggcctgctgtacgtgtacgagagactgacctggaccaccaaggccaaagagcgggccttcaagcggcagtttgtggaacacgcctccgagaaactgcagctggtgatcagctacaccggcagcaattgcagccaccaggtgcagcaagagctgtccggaacattcgcccacctgtgtcagcaggtcgacgtgaccagagagaacctggaacaagagatcgccgccatgaataagaaaatcgaggtgctcgactccctgcagagcaaggccaagctgctgagaaacaaggccggctggctggacagcgagctgaatatgttcacccaccagtacctgcagcctagccgctaataatgtacaagtaaagcgcccgcaggcttgcacccgtacctttgggagcgcgcgccctcgtcgtgtcgtgacgtcacccgttctgttggatccctggaggcttgctgaaggctgtatgctgttcttataaaccttgaggactgttttggccactgactgacagtcctcagtttataagaacaggacacaaggcctgttactagcactcacatggaacaaatggcccagatctagggtggggccacctgccggtaggtgtgcggtaggcttttctccgtcgcaggacgcagggttcgggcctagcttaagcttctcgagatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgcccttctgctacgtcccttcggccctcaatccagcggaccttccttcccgcggcctgctgccggctctgcggcctcttccgcgtcttcgccttcgccctcagacgagtcggatctccctttgggccgcctccccgcctgctgatcgccctgagcctggccctggtgaccaacagccaggtgcaattgctagagtcgactctagagcggccgcctaaggctcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 2 - protein, 757 aa, Artificial Sequence
  Synthetic Construct
MSLLFSRCNSIVTVKKNKRHMAEVNASPLKHFVTAKKKINGIFEQLGAYIQESATFLEDTYRNAELDPVTTEEQVLDVKGYLSKVRGISEVLARRHMKVAFFGRTSNGKSTVINAMLWDKVLPSGIGHTTNCFLRVEGTDGHEAFLLTEGSEEKRSAKTVNQLAHALHQDKQLHAGSLVSVMWPNSKCPLLKDDLVLMDSPGIDVTTELDSWIDKFCLDADVFVLVANSESTLMQTEKHFFHKVSERLSRPNIFILNNRWDASASEPEYMEEVRRQHMERCTSFLVDELGVVDRSQAGDRIFFVSAKEVLNARIQKAQGMPEGGGALAEGFQVRMFEFQNFERRFEECISQSAVKTKFEQHTVRAKQIAEAVRLIMDSLHMAAREQQVYCEEMREERQDRLKFIDKQLELLAQDYKLRIKQITEEVERQVSTAMAEEIRRLSVLVDDYQMDFHPSPVVLKVYKNELHRHIEEGLGRNMSDRCSTAITNSLQTMQQDMIDGLKPLLPVSVRSQIDMLVPRQCFSLNYDLNCDKLCADFQEDIEFHFSLGWTMLVNRFLGPKNSRRALMGYNDQVQRPIPLTPANPSMPPLPQGSLTQEEFMVSMVTGLASLTSRTSMGILVVGGVVWKAVGWRLIALSFGLYGLLYVYERLTWTTKAKERAFKRQFVEHASEKLQLVISYTGSNCSHQVQQELSGTFAHLCQQVDVTRENLEQEIAAMNKKIEVLDSLQSKAKLLRNKAGWLDSELNMFTHQYLQPSR

SEQ ID NO: 3 - DNA, 4560 bp, Artificial Sequence
  Vector Genome Syn.PI.rMnf2opt.link.rMfn2 miR1518 WPRE.BGH
  repeat_region (1)..(141): 5′ITR
  promoter (223)..(682): hSynapsin
  Intron (694)..(826): Promega Chimeric Intron
  CDS (863)..(3133): rMfn2 engineered
  misc_feature (3134)..(3139): Dbl stop codon
  misc_feature (3140)..(3229): linker
  misc_signal (3230)..(3252): 5′flank
  misc_feature (3253)..(3316): rMfn2 miR1518
  misc_signal (3317)..(3360): 3′flank
  enhancer (3457)..(4044): WRPE
  misc_feature (4385)..(4430): Additional AAV sequences
  repeat_region (4431)..(4560): 3′ITR
ccttaattaggctgcgcgctcgctcgctcactgaggccgggcgaccaaaggtcgcccgacgcccgggctttgcccgggcggcctcagtgagcgagcgagcgcgcagagagggagtggccaactccatcactaggggttccttgtagttaatgattaacccgccatgctacttatctacgtagccatgctctaggaagatcggaattcgcccttaagctagactcgagctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcggtaagtatcaaggttacaagacaggtttaaggagaccaatagaaactgggcttgtcgagacagagaagactcttgcgtttctgataggcacctattggtcttactgacatccactttgcctttctctccacagtacgtagaattcacgcgtgctagcaccggtgccaccatgtctctgctgttcagccggtgcaacagcatcgtgaccgtgaagaaagacaagaggcacatggccgaagtgaacgcctctccactgaagcacttcgtgaccgccaagaagaagatcaacggcatcttcgagcagctgggcgcctacatccaagagtctgccggcttcctggaagatacccacagaaacaccgagctggaccccgtgaccacagaggaacaggtgctggacgtgaagggctacctgtctaaagtgcggggcatcagcgaggtgctggctagaaggcatatgaaggtggcctttttcggcaggaccagcaacggcaagagcaccgtgatcaacgccatgctgtgggacaaagtgctgccttctggcatcggccacaccaccaactgctttctgagagtcggcggcacagacggccacgaggcttttttgctgaccgagggctccgaagagaagaaatccgtgaaaaccgtgaaccagctggcccacgctctgcaccaggatgaacaactgcacgccggaagcctggtgtccgtgatgtggcctaacagcaagtgccctctgctgaaggacgacctggtgctgatggactctcctggaatcgacgtgaccactgagctggacagctggatcgacaagttctgcctggacgccgacgtgttcgtgctggtggctaactctgagagcaccctgatgcagaccgagaagcagttcttccacaaggtgtccgagaggctgagcaggcccaatatctttatcctgaacaacagatgggacgccagcgccagcgagcccgagtatatggaagaagtgcggaggcagcatatggaaaggtgtaccagcttcctggtggacgagctgggagtcgtggatagagcacaggccggcgacagaatcttcttcgtgtctgccaaagaggtgctgagcgccagggtgcagaaagcccagggaatgcctgaaggcggcggagctttggccgagggatttcaagtgcggatgttcgagttccagaacttcgagcggagattcgaggaatgcatcagccagagcgccgtcaagacaaagttcgagcagcacacagtgcgggccaagcagatcgctgaggctgtgcggctgatcatggacagcctgcatatcgccgctcaagagcagagggtgtactgcctggaaatgcgggaagagaggcaggacaggctgagattcatcgacaagcagctggaactgctggcccaggactacaagctgaggatcaagcagatgaccgaagaggtggaaagacaggtgtccaccgccatggccgaggaaatccgtagactgtctgtgctggtcgacgagtaccagatggactttcacccctctccagtggtgctgaaggtctacaaaaacgagctgcacaggcacatcgaggaaggcctgggcagaaacatgagcgacaggtgctctaccgctatcgccagcagcctgcagacaatgcagcaggacatgatcgacggcctgaagcctctgctgcctgtgtctgtgcggaaccagatcgatatgctggtgcccaggcagtgcttctccctgtcctacgacctgaactgcgacaagctgtgtgccgacttccaagaggacatcgagtttcacttcagcctcggctggacaatgctggtcaacagattcctgggacctaagaactccagacgggccctgctgggatacaacgatcaggtgcaaaggcccctgcctctgacaccagctaacccttctatgcctcctctgcctcaaggctctctgacccaagaggaactgatggtgtccatggtcacaggcctggcttccctgaccagcagaacctctatgggcatcctggtcgtcggcggagttgtgtggaaagctgttggctggcgcctgatcgccctgagctttggactgtacggactgctgtacgtgtacgagagactgacctggaccaccagggccaaagagagggccttcaagaggcagttcgtggaatacgcctccgagaaactgcagctgattatctcctacaccggcagcaactgcagccaccaggtgcagcaagagctgtctggaacattcgcccacctgtgccagcaggtcgacatcaccagagacaacctggaacaagagatcgccgctatgaataagaaggtggaagccctggactccctgcagagcaaggctaagctgctgagaaacaaggccggctggctggactccgagctgaatatgtttatccaccagtacctgcagcctagccgctgatgatgtacaagtaaagcggccgcaagcttgcacccgtacctttgggagcgcgcgccctcgtcgtgtcgtgacgtcacccgttctgttggatccctggaggcttgctgaaggctgtatgctgttcttataaaccttgaggacagttttggccactgactgactgtcctcagtttataagaacaggacacaaggcctgttactagcactcacatggaacaaatggcccagatctagggtggggccacctgccggtaggtgtgcggtaggcttttctccgtcgcaggacgcagggttcgggcctagcttctcgagatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgcccttctgctacgtcccttcggccctcaatccagcggaccttccttcccgcggcctgctgccggctctgcggcctcttccgcgtcttcgccttcgccctcagacgagtcggatctccctttgggccgcctccccgcctgctgatcgccctgagcctggccctggtgaccaacagccaggtgcaattgaagctttctagagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 4 - protein, 757 aa, Artificial Sequence
  Synthetic Construct
MSLLFSRCNSIVTVKKDKRHMAEVNASPLKHFVTAKKKINGIFEQLGAYIQESAGFLEDTHRNTELDPVTTEEQVLDVKGYLSKVRGISEVLARRHMKVAFFGRTSNGKSTVINAMLWDKVLPSGIGHTTNCFLRVGGTDGHEAFLLTEGSEEKKSVKTVNQLAHALHQDEQLHAGSLVSVMWPNSKCPLLKDDLVLMDSPGIDVTTELDSWIDKFCLDADVFVLVANSESTLMQTEKQFFHKVSERLSRPNIFILNNRWDASASEPEYMEEVRRQHMERCTSFLVDELGVVDRAQAGDRIFFVSAKEVLSARVQKAQGMPEGGGALAEGFQVRMFEFQNFERRFEECISQSAVKTKFEQHTVRAKQIAEAVRLIMDSLHIAAQEQRVYCLEMREERQDRLRFIDKQLELLAQDYKLRIKQMTEEVERQVSTAMAEEIRRLSVLVDEYQMDFHPSPVVLKVYKNELHRHIEEGLGRNMSDRCSTAIASSLQTMQQDMIDGLKPLLPVSVRNQIDMLVPRQCFSLSYDLNCDKLCADFQEDIEFHFSLGWTMLVNRFLGPKNSRRALLGYNDQVQRPLPLTPANPSMPPLPQGSLTQEELMVSMVTGLASLTSRTSMGILVVGGVVWKAVGWRLIALSFGLYGLLYVYERLTWTTRAKERAFKRQFVEYASEKLQLIISYTGSNCSHQVQQELSGTFAHLCQQVDITRDNLEQEIAAMNKKVEALDSLQSKAKLLRNKAGWLDSELNMFIHQYLQPSR

SEQ ID NO: 5 - DNA, 141 bp, Artificial Sequence
  5′ ITR
ccttaattaggctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 6 - DNA, 460 bp, Artificial Sequence
  hSynapsin promoter
tcgagctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgt

SEQ ID NO: 7 - DNA, 133 bp, Artificial Sequence
  Promega Chimeric Intron
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 8 - DNA, 583 bp, Artificial Sequence
  WPRE
tcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgcccttctgctacgtcccttcggccctcaatccagcggaccttccttcccgcggcctgctgccggctctgcggcctcttccgcgtcttcgccttcgccctcagacgagtcggatctccctttgggccgcctcc

SEQ ID NO: 9 - DNA, 46 bp, Artificial Sequence
  additonal AAV sequences
catggctacgtagataagtagcatggcgggttaatcattaactaca

SEQ ID NO: 10 - DNA, 130 bp, Artificial Sequence
  3′ ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 11 - DNA, 2274 bp, Artificial Sequence
  human Mfn2 engineered nucleic acid seuquence encoding mitofusin 2 protein
atgagcctgctgttcagccggtgcaacagcatcgtgaccgtgaagaaaaacaagcggcacatggccgaagtgaacgcctctccactgaagcacttcgtgaccgccaagaagaagatcaacggcatcttcgagcagctgggcgcctacatccaagagagcgccaccttcctggaagatacctacagaaacgccgagctggaccccgtgaccacagaggaacaggtgctggatgtgaagggctacctgagcaaagtgcggggcatctctgaagtgctggccagacggcatatgaaggtggcctttttcggccggaccagcaacggcaagagcaccgtgatcaatgccatgctgtgggacaaagtgctgccctctggaatcggccacaccaccaactgctttctgagagtggaaggcaccgacggccacgaggcatttctgctgacagagggctccgaggaaaagcggagcgccaagacagtgaaccagctggcccatgctctgcaccaggataagcagctgcatgccggaagcctggtgtccgtgatgtggcccaatagcaagtgccctctgctgaaggacgacctggtgctgatggatagccccgggatcgatgtgaccaccgaactggacagctggatcgacaagttctgcctggacgccgacgtgttcgtgctggtggccaatagcgagagcaccctgatgcagaccgagaagcactttttccacaaggtgtccgagcggctgagcagacccaatatctttatcctgaacaacagatgggacgccagcgccagcgagcccgagtatatggaagaagtgcggcggcagcatatggaacggtgcacatcctttctggtggacgagctgggcgtcgtggatagatctcaggccggcgacagaatcttctttgtgtccgccaaagaggtgctgaacgcccggattcagaaagcccagggaatgcctgaaggcggaggtgcactggccgagggatttcaagtgcggatgttcgagttccagaacttcgagcggagattcgaggagtgcatcagccagagcgccgtcaagaccaagttcgagcagcataccgtgcgggccaagcagattgccgaagccgtcagactgatcatggacagcctgcatatggccgccagagagcagcaggtctactgcgaggaaatgcgggaagagagacaggaccggctgaagttcatcgacaagcagctggaactgctggcccaggactacaagctgcggatcaagcagatcaccgaagaggtggaaagacaggtgtccaccgctatggccgaggaaatcagacggctgagcgtgctggtcgacgactaccagatggactttcacccctctccagtggtgctgaaagtctacaaaaacgagctgcaccggcacatcgaggaaggcctgggcagaaacatgagcgacagatgcagcaccgccatcaccaatagcctgcagaccatgcagcaggacatgatcgacggcctgaaacctctgctgcctgtgtccgtcagatcccagatcgatatgctggtgccccggcagtgctttagcctgaactacgacctgaactgcgacaagctgtgcgccgatttccaagaggacatcgagtttcacttcagcctcggctggacaatgctggtcaacagatttctgggccccaagaacagcagacgggccctgatgggctacaacgatcaggtgcagaggcccattcctctgacacccgccaatcctagcatgcctccactgcctcagggcagcctgacacaagaggaattcatggtgtctatggtcaccggcctggccagcctgaccagcagaacatctatgggcatcctggtcgtcggcggcgttgtgtggaaagctgttggctggcggctgatcgccctgagctttggcctgtatggcctgctgtacgtgtacgagagactgacctggaccaccaaggccaaagagcgggccttcaagcggcagtttgtggaacacgcctccgagaaactgcagctggtgatcagctacaccggcagcaattgcagccaccaggtgcagcaagagctgtccggaacattcgcccacctgtgtcagcaggtcgacgtgaccagagagaacctggaacaagagatcgccgccatgaataagaaaatcgaggtgctcgactccctgcagagcaaggccaagctgctgagaaacaaggccggctggctggacagcgagctgaatatgttcacccaccagtacctgcagcctagccgctaa

SEQ ID NO: 12 - DNA, 2271 bp, Artificial Sequence
  rMfn2 engineered nucleic acid sequence encoding mitofusin 2 protein
atgtctctgctgttcagccggtgcaacagcatcgtgaccgtgaagaaagacaagaggcacatggccgaagtgaacgcctctccactgaagcacttcgtgaccgccaagaagaagatcaacggcatcttcgagcagctgggcgcctacatccaagagtctgccggcttcctggaagatacccacagaaacaccgagctggaccccgtgaccacagaggaacaggtgctggacgtgaagggctacctgtctaaagtgcggggcatcagcgaggtgctggctagaaggcatatgaaggtggcctttttcggcaggaccagcaacggcaagagcaccgtgatcaacgccatgctgtgggacaaagtgctgccttctggcatcggccacaccaccaactgctttctgagagtcggcggcacagacggccacgaggcttttttgctgaccgagggctccgaagagaagaaatccgtgaaaaccgtgaaccagctggcccacgctctgcaccaggatgaacaactgcacgccggaagcctggtgtccgtgatgtggcctaacagcaagtgccctctgctgaaggacgacctggtgctgatggactctcctggaatcgacgtgaccactgagctggacagctggatcgacaagttctgcctggacgccgacgtgttcgtgctggtggctaactctgagagcaccctgatgcagaccgagaagcagttcttccacaaggtgtccgagaggctgagcaggcccaatatctttatcctgaacaacagatgggacgccagcgccagcgagcccgagtatatggaagaagtgcggaggcagcatatggaaaggtgtaccagcttcctggtggacgagctgggagtcgtggatagagcacaggccggcgacagaatcttcttcgtgtctgccaaagaggtgctgagcgccagggtgcagaaagcccagggaatgcctgaaggcggcggagctttggccgagggatttcaagtgcggatgttcgagttccagaacttcgagcggagattcgaggaatgcatcagccagagcgccgtcaagacaaagttcgagcagcacacagtgcgggccaagcagatcgctgaggctgtgcggctgatcatggacagcctgcatatcgccgctcaagagcagagggtgtactgcctggaaatgcgggaagagaggcaggacaggctgagattcatcgacaagcagctggaactgctggcccaggactacaagctgaggatcaagcagatgaccgaagaggtggaaagacaggtgtccaccgccatggccgaggaaatccgtagactgtctgtgctggtcgacgagtaccagatggactttcacccctctccagtggtgctgaaggtctacaaaaacgagctgcacaggcacatcgaggaaggcctgggcagaaacatgagcgacaggtgctctaccgctatcgccagcagcctgcagacaatgcagcaggacatgatcgacggcctgaagcctctgctgcctgtgtctgtgcggaaccagatcgatatgctggtgcccaggcagtgcttctccctgtcctacgacctgaactgcgacaagctgtgtgccgacttccaagaggacatcgagtttcacttcagcctcggctggacaatgctggtcaacagattcctgggacctaagaactccagacgggccctgctgggatacaacgatcaggtgcaaaggcccctgcctctgacaccagctaacccttctatgcctcctctgcctcaaggctctctgacccaagaggaactgatggtgtccatggtcacaggcctggcttccctgaccagcagaacctctatgggcatcctggtcgtcggcggagttgtgtggaaagctgttggctggcgcctgatcgccctgagctttggactgtacggactgctgtacgtgtacgagagactgacctggaccaccagggccaaagagagggccttcaagaggcagttcgtggaatacgcctccgagaaactgcagctgattatctcctacaccggcagcaactgcagccaccaggtgcagcaagagctgtctggaacattcgcccacctgtgccagcaggtcgacatcaccagagacaacctggaacaagagatcgccgctatgaataagaaggtggaagccctggactccctgcagagcaaggctaagctgctgagaaacaaggccggctggctggactccgagctgaatatgtttatccaccagtacctgcagcctagccgc

SEQ ID NO: 13 - DNA, 23 bp, Artificial Sequence
  5′flank
ctggaggcttgctgaaggctgta

SEQ ID NO: 14 - DNA, 44 bp, Artificial Sequence
  3′flank
caggacacaaggcctgttactagcactcacatggaacaaatggc

SEQ ID NO: 15 - DNA, 64 bp, Artificial Sequence
  hMfn2 miR1693
tgctgttcttataaaccttgaggactgttttggccactgactgacagtcctcagtttataagaa

SEQ ID NO: 16 - DNA, 64 bp, Artificial Sequence
  rMfn2 miR1518
tgctgttcttataaaccttgaggacagttttggccactgactgactgtcctcagtttataagaa

SEQ ID NO: 17 - DNA, 90 bp, Artificial Sequence
  linker
tgtacaagtaaagcgcccgcaggcttgcacccgtacctttgggagcgcgcgccctcgtcgtgtcgtgacgtcacccgttctgttggatcc

SEQ ID NO: 18 - DNA, 2274 bp, Artificial Sequence
  hMfn2 endogenous nucleic acid sequence
  CDS (1)..(2274)
atgtccctgctcttctctcgatgcaactctatcgtcacagtcaagaaaaataagagacacatggctgaggtgaatgcatccccacttaagcactttgtcactgccaagaagaagatcaatggcatttttgagcagctgggggcctacatccaggagagcgccaccttccttgaagacacgtacaggaatgcagaactggaccccgttaccacagaagaacaggttctggacgtcaaaggttacctatccaaagtgagaggcatcagtgaggtgctggctcggaggcacatgaaagtggctttttttggccggacgagcaatgggaagagcaccgtgatcaatgccatgctctgggacaaagttctgccctctgggattggccacaccaccaattgcttcctgcgggtagagggcacagatggccatgaggcctttctccttaccgagggctcagaggaaaagaggagtgccaagactgtgaaccagctggcccatgccctccaccaggacaagcagctccatgccggcagcctagtgagtgtgatgtggcccaactctaagtgcccacttctgaaggatgacctcgttttgatggacagccctggtattgatgtcaccacagagctggacagctggattgacaagttttgtctggatgctgatgtgtttgtgctggtggccaactcagagtccaccctgatgcagacggaaaagcacttcttccacaaggtgagtgagcgtctctcccggccaaacatcttcatcctgaacaaccgctgggatgcatctgcctcagagcccgagtacatggaggaggtgcggcggcagcacatggagcgttgtaccagcttcctggtggatgagctgggcgtggtggatcgatcccaggccggggaccgcatcttctttgtgtctgctaaggaggtgctcaacgccaggattcagaaagcccagggcatgcctgaaggagggggcgctctcgcagaaggctttcaagtgaggatgtttgagtttcagaattttgagaggagatttgaggagtgcatctcccagtctgcagtgaagaccaagtttgagcagcacacggtccgggccaagcagattgcagaggcggttcgactcatcatggactccctgcacatggcggctcgggagcagcaggtttactgcgaggaaatgcgtgaagagcggcaagaccgactgaaatttattgacaaacagctggagctcttggctcaagactataagctgcgaattaagcagattacggaggaagtggagaggcaggtgtcgactgcaatggccgaggagatcaggcgcctctctgtactggtggacgattaccagatggacttccacccttctccagtagtcctcaaggtttataagaatgagctgcaccgccacatagaggaaggactgggtcgaaacatgtctgaccgctgctccacggccatcaccaactccctgcagaccatgcagcaggacatgatagatggcttgaaacccctccttcctgtgtctgtgcggagtcagatagacatgctggtcccacgccagtgcttctccctcaactatgacctaaactgtgacaagctgtgtgctgacttccaggaagacattgagttccatttctctctcggatggaccatgctggtgaataggttcctgggccccaagaacagccgtcgggccttgatgggctacaatgaccaggtccagcgtcccatccctctgacgccagccaaccccagcatgcccccactgccacagggctcgctcacccaggaggagttcatggtttccatggttaccggcctggcctccttgacatccaggacctccatgggcattcttgttgttggaggagtggtgtggaaggcagtgggctggcggctcattgccctctcctttgggctctatggcctcctctacgtctatgagcgtctgacctggaccaccaaggccaaggagagggccttcaagcgccagtttgtggagcatgccagcgagaagctgcagcttgtcatcagctacactggctccaactgcagccaccaagtccagcaggaactgtctgggacctttgctcatctgtgtcagcaagttgacgtcacccgggagaacctggagcaggaaattgccgccatgaacaagaaaattgaggttcttgactcacttcagagcaaagcaaagctgctcaggaataaagccggttggttggacagtgagctcaacatgttcacacaccagtacctgcagcccagcagatag

SEQ ID NO: 19 - protein, 757 aa, Artificial Sequence; identical to SEQ ID NO: 2
  Synthetic Construct

SEQ ID NO: 20 - DNA, 2274 bp, Artificial Sequence
  rMfn2 endogenous nucleic acid sequence
  CDS (1)..(2274)
atgtccctgctcttttctcgatgcaactccatcgtcaccgtcaagaaggataagcgacacatggctgaggtgaatgcttcccctctcaagcactttgtcactgccaagaaaaagatcaatggcatctttgagcagctgggggcctacatccaagagagtgccggcttccttgaagacacccacaggaatacagaactggacccagttactacagaagagcaggtcctggatgtcaaagggtacctgtccaaggtcaggggaatcagcgaggtgctggccaggcggcacatgaaggtggctttttttggccggacgagcaatgggaagagcaccgtgatcaatgccatgctttgggacaaagttctgccatctgggattggccacaccaccaattgcttcctgcgggttgggggcacggatggccacgaggccttcctcctcacggagggctcagaagagaagaagagtgtcaagaccgtgaaccagctagcccatgccctccatcaggatgagcagctgcatgcaggcagcctggtgagtgtgatgtggcccaactccaagtgtccacttctgaaggatgacctcgtgctgatggacagccctgggatcgatgtcaccacggagctggacagctggattgataagttttgcctggatgctgatgtgtttgtactggtggccaactcagagtccacgctgatgcagacggagaagcagttcttccacaaagtgagtgagcgtctctcccggcccaacatcttcatcctgaacaaccgctgggatgcgtctgcctcggagcctgagtacatggaggaggtgcggcggcagcacatggaacgctgcaccagctttctggtggatgagctaggcgtggtggatcgagctcaggccggggaccggatcttctttgtgtctgccaaggaggtgctcagtgccagggtccagaaagcccagggcatgccagaaggaggtggcgctcttgcagaaggttttcaagtgaggatgtttgagtttcagaatttcgagaggcgatttgaggagtgcatttcccagtctgcagtaaagaccaaatttgagcagcacacagtccgggccaagcagattgcagaggccgtccgtctcatcatggattccctgcacattgcggctcaggagcagcgggtttattgtcttgaaatgcgggaagagcggcaagaccggctgagattcattgacaagcagctggagctcctggctcaagactataagctacggattaagcagatgacagaggaagtggaaaggcaggtgtccacagccatggctgaagagatcaggcgcctctctgtgctggttgacgagtaccagatggactttcacccatccccagttgtcctcaaggtttataagaatgaactgcaccgccatatagaggaaggcctgggacgaaacatgtctgaccgctgctccacagccattgccagttcactgcagactatgcagcaagacatgatagacggcttgaagccccttcttcctgtgtctgtgcggaatcagatagacatgctggtccctcgacagtgtttctccctcagctatgacctgaactgtgacaagctgtgcgctgactttcaggaggacatcgaattccatttctcccttggatggactatgctagtgaataggttcctgggccctaagaatagccggcgggccttgctgggctacaatgatcaggttcagcgtcctctccctctgacacctgccaaccccagcatgccacccttgccacagggctccctcacccaggaggagctcatggtctccatggttactggcctggcctctctgacttccaggacctctatgggcattctcgtggtcggaggagtggtgtggaaggcagtgggctggagactcatcgccctctcctttggactgtatggcctcctgtacgtgtatgagcggctgacctggaccaccagagccaaggagagggccttcaagcgccagtttgtggagtatgccagtgagaagctgcagctcattatcagttataccggctctaactgcagccaccaagttcagcaagaattgtctgggacatttgctcatctgtgccagcaagttgacatcacccgggataatctggagcaggaaattgctgccatgaacaagaaagttgaggctctggattcacttcagagcaaagccaaattgctcaggaataaagctggctggttggacagcgaactcaacatgttcatacaccagtacctgcagcccagcagatag

SEQ ID NO: 21 - protein, 757 aa, Artificial Sequence; identical to SEQ ID NO: 4
  Synthetic Construct

SEQ ID NO: 22 - DNA, 215 bp, Artificial Sequence
  bGH polyA
gcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 23 - DNA, 232 bp, Artificial Sequence
  SV40 polyA
cgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcg

SEQ ID NO: 24 - DNA, 2315 bp, Artificial Sequence
  hMfn2 engineered nucleic acid sequnece V1
  CDS (1)..(2274)
gctagcaccggtgccaccatgagcctgctgttcagccggtgcaacagcatcgtgaccgtgaagaaaaacaagcggcacatggccgaagtgaacgcctctccactgaagcacttcgtgaccgccaagaagaagatcaacggcatcttcgagcagctgggcgcctacatccaagagagcgccaccttcctggaagatacctacagaaacgccgagctggaccccgtgaccacagaggaacaggtgctggatgtgaagggctacctgagcaaagtgcggggcatctctgaagtgctggccagacggcatatgaaggtggcctttttcggccggaccagcaacggcaagagcaccgtgatcaatgccatgctgtgggacaaagtgctgccctctggaatcggccacaccaccaactgctttctgagagtggaaggcaccgacggccacgaggcatttctgctgacagagggctccgaggaaaagcggagcgccaagacagtgaaccagctggcccatgctctgcaccaggataagcagctgcatgccggaagcctggtgtccgtgatgtggcccaatagcaagtgccctctgctgaaggacgacctggtgctgatggatagccccgggatcgatgtgaccaccgaactggacagctggatcgacaagttctgcctggacgccgacgtgttcgtgctggtggccaatagcgagagcaccctgatgcagaccgagaagcactttttccacaaggtgtccgagcggctgagcagacccaacaattttatcctgaacaacagatgggacgccagcgccagcgagcccgagtatatggaagaagtgcggcggcagcatatggaacggtgcacatcctttctggtggacgagctgggcgtcgtggatagatctcaggccggcgacagaatcttctttgtgtccgccaaagaggtgctgaacgcccggattcagaaagcccagggaatgcctgaaggcggaggtgcactggccgagggatttcaagtgcggatgttcgagttccagaacttcgagcggagattcgaggaatgcatcagccagagcgccgtcaagaccaagtttgagcagcataccgtgcgggccaagcagattgccgaagccgtcagactgatcatggacagcctgcatatggccgccagagagcagcaggtctactgcgaggaaatgcgggaagagagacaggaccggctgaagttcatcgacaagcagctggaactgctggcccaggactacaagctgcggatcaagcagatcaccgaagaggtggaaagacaggtgtccaccgctatggccgaggaaatcagacggctgagcgtgctggtcgacgactaccagatggactttcacccctctccagtggtgctgaaggtctacaagaacgagctgcaccggcacatcgaggaaggcctgggcagaaacatgagcgacagatgcagcaccgccatcaccaatagcctgcagaccatgcagcaggacatgatcgacggcctgaaacctctgctgcctgtgtccgtcagatcccagatcgatatgctggtgccccggcagtgctttagcctgaactacgacctgaactgcgacaagctgtgcgccgatttccaagaggacatcgagtttcacttcagcctcggctggacaatgctggtcaacagatttctgggccccaagaacagcagacgggccctgatgggctacaacgatcaggtgcagaggcccattcctctgacacccgccaatcctagcatgcctccactgcctcagggcagcctgacacaagaggaattcatggtgtctatggtcaccggcctggccagcctgaccagcagaacatctatgggcatcctggtcgtcggcggcgttgtgtggaaagctgttggctggcggctgatcgccctgagctttggcctgtatggcctgctgtacgtgtacgagagactgacctggaccaccaaggccaaagagcgggccttcaagcggcagtttgtggaacacgcctccgagaaactgcagctggtgatcagctacaccggcagcaattgcagccaccaggtgcagcaagagctgtccggaacattcgcccacctgtgtcagcaggtcgacgtgaccagagagaacctggaacaagagatcgccgccatgaacaagaaaatcgaggtgctcgactccctgcagagcaaggccaagctgctgagaaacaaggccggctggctggacagcgagctgaatatgttcacccaccagtacctgcagcctagccgctaataatgtacagcggccgcaagctt

SEQ ID NO: 25 - protein, 758 aa, Artificial Sequence
  Synthetic Construct
ASTGATMSLLFSRCNSIVTVKKNKRHMAEVNASPLKHFVTAKKKINGIFEQLGAYIQESATFLEDTYRNAELDPVTTEEQVLDVKGYLSKVRGISEVLARRHMKVAFFGRTSNGKSTVINAMLWDKVLPSGIGHTTNCFLRVEGTDGHEAFLLTEGSEEKRSAKTVNQLAHALHQDKQLHAGSLVSVMWPNSKCPLLKDDLVLMDSPGIDVTTELDSWIDKFCLDADVFVLVANSESTLMQTEKHFFHKVSERLSRPNNFILNNRWDASASEPEYMEEVRRQHMERCTSFLVDELGVVDRSQAGDRIFFVSAKEVLNARIQKAQGMPEGGGALAEGFQVRMFEFQNFERRFEECISQSAVKTKFEQHTVRAKQIAEAVRLIMDSLHMAAREQQVYCEEMREERQDRLKFIDKQLELLAQDYKLRIKQITEEVERQVSTAMAEEIRRLSVLVDDYQMDFHPSPVVLKVYKNELHRHIEEGLGRNMSDRCSTAITNSLQTMQQDMIDGLKPLLPVSVRSQIDMLVPRQCFSLNYDLNCDKLCADFQEDIEFHFSLGWTMLVNRFLGPKNSRRALMGYNDQVQRPIPLTPANPSMPPLPQGSLTQEEFMVSMVTGLASLTSRTSMGILVVGGVVWKAVGWRLIALSFGLYGLLYVYERLTWTTKAKERAFKRQFVEHASEKLQLVISYTGSNCSHQVQQELSGTFAHLCQQVDVTRENLEQEIAAMNKKIEVLDSLQSKAKLLRNKAGWLDSELNMFTHQY

SEQ ID NO: 26 - DNA, 21 bp, Artificial Sequence
  antisense sequence
aaaccttgaggactactggag

SEQ ID NO: 27 - DNA, 21 bp, Artificial Sequence
  antisense sequence
ttgacgtccagaacctgttct

SEQ ID NO: 28 - DNA, 21 bp, Artificial Sequence
  antisense sequence
agaagtgggcacttagagttg

SEQ ID NO: 29 - DNA, 21 bp, Artificial Sequence
  antisense sequence
ttcagaagtgggcacttagag

SEQ ID NO: 30 - DNA, 21 bp, Artificial Sequence
  antisense sequence
ttgtcaatccagctgtccagc

SEQ ID NO: 31 - DNA, 21 bp, Artificial Sequence
  antisense sequence
caaacttggtcttcactgcag

SEQ ID NO: 32 - DNA, 21 bp, Artificial Sequence
  antisense sequence
aaaccttgaggactactggag

SEQ ID NO: 33 - DNA, 21 bp, Artificial Sequence
  antisense sequence
taaccatggaaaccatgaact

SEQ ID NO: 34 - DNA, 21 bp, Artificial Sequence
  antisense sequence
acaacaagaatgcccatggag

SEQ ID NO: 35 - DNA, 21 bp, Artificial Sequence
  antisense sequence
aaaggtcccagacagttcctg

SEQ ID NO: 36 - DNA, 21 bp, Artificial Sequence
  antisense sequence
tgttcatggcggcaatttcct

SEQ ID NO: 37 - DNA, 21 bp, Artificial Sequence
  antisense sequence
tgaggttggctattgattgac

SEQ ID NO: 38 - DNA, 21 bp, Artificial Sequence
  antisense sequence
ttctcacacagtcaacacctt

SEQ ID NO: 39 - DNA, 21 bp, Artificial Sequence
  antisense sequence
tttcctcgcagtaaacctgct

SEQ ID NO: 40 - DNA, 21 bp, Artificial Sequence
  antisense sequence
agaaatggaactcaatgtctt

SEQ ID NO: 41 - DNA, 21 bp, Artificial Sequence
  antisense sequence
tgaacaggacatcacctgtga

SEQ ID NO: 42 - DNA, 21 bp, Artificial Sequence
  antisense sequence
aatacaagcaggtatgtgaac

SEQ ID NO: 43 - DNA, 21 bp, Artificial Sequence
  antisense sequence
taaacctgctgctcccgagcc

SEQ ID NO: 44 - DNA, 21 bp, Artificial Sequence
  antisense sequence
tagaggaggccatagagccca

SEQ ID NO: 45 - DNA, 21 bp, Artificial Sequence
  antisense sequence
tctacccgcaggaagcaattg

SEQ ID NO: 46 - DNA, 21 bp, Artificial Sequence
  antisense sequence
ctccttagcagacacaaagaa

SEQ ID NO: 47 - DNA, 21 bp, Artificial Sequence
  Target hMfn2 Sequence
agaacaggttctggacgtcaa

SEQ ID NO: 48 - DNA, 21 bp, Artificial Sequence
  Target hMfn2 Sequence
caactctaagtgcccacttct

SEQ ID NO: 49 - DNA, 21 bp, Artificial Sequence
  Target hMfn2 Sequence
ctctaagtgcccacttctgaa

SEQ ID NO: 50 - DNA, 21 bp, Artificial Sequence
  Target hMfn2 Sequence
gctggacagctggattgacaa

SEQ ID NO: 51 - DNA, 21 bp, Artificial Sequence
  Target hMfn2 Sequence
ctgcagtgaagaccaagtttg

SEQ ID NO: 52 - DNA, 21 bp, Artificial Sequence
  Target hMfn2 Sequence
ctccagtagtcctcaaggttt

SEQ ID NO: 53 - DNA, 21 bp, Artificial Sequence
  Target hMfn2 Sequence
agttcatggtttccatggtta

SEQ ID NO: 54 - DNA, 21 bp, Artificial Sequence
  Target hMfn2 Sequence
ctccatgggcattcttgttgt

SEQ ID NO: 55 - DNA, 21 bp, Artificial Sequence
  Target hMfn2 Sequence
caggaactgtctgggaccttt

SEQ ID NO: 56 - DNA, 21 bp, Artificial Sequence
  Target hMfn2 Sequence
aggaaattgccgccatgaaca

SEQ ID NO: 57 - DNA, 21 bp, Artificial Sequence
  Target hMfn2 Sequence
agtcctcaaggtttataagaa

SEQ ID NO: 58 - DNA, 21 bp, Artificial Sequence
  Target hMfn2 Sequence
gtcaatcaatagccaacctca

SEQ ID NO: 59 - DNA, 21 bp, Artificial Sequence
  Target hMfn2 Sequence
aaggtgttgactgtgtgagaa

SEQ ID NO: 60 - DNA, 21 bp, Artificial Sequence
  Target hMfn2 Sequence
agcaggtttactgcgaggaaa

SEQ ID NO: 61 - DNA, 21 bp, Artificial Sequence
  Target hMfn2 Sequence
aagacattgagttccatttct

SEQ ID NO: 62 - DNA, 21 bp, Artificial Sequence
  Target hMfn2 Sequence
tcacaggtgatgtcctgttca

SEQ ID NO: 63 - DNA, 21 bp, Artificial Sequence
  Target hMfn2 Sequence
gttcacatacctgcttgtatt

SEQ ID NO: 64 - DNA, 21 bp, Artificial Sequence
  Target hMfn2 Sequence
ggctcgggagcagcaggttta

SEQ ID NO: 65 - DNA, 21 bp, Artificial Sequence
  Target hMfn2 Sequence
tgggctctatggcctcctcta

SEQ ID NO: 66 - DNA, 21 bp, Artificial Sequence
  Target hMfn2 Sequence
caattgcttcctgcgggtaga

SEQ ID NO: 67 - DNA, 21 bp, Artificial Sequence
  Target hMfn2 Sequence
ttctttgtgtctgctaaggag

SEQ ID NO: 68 - DNA, 21 bp, Artificial Sequence
  miR1693(21-mer)
ttcttataaaccttgaggact

SEQ ID NO: 69 - DNA, 4771 bp, Artificial Sequence
  Vector genome CAG.CI.hMfn2.GA.link.miR538.WPRE. SV40
  repeat_region (1)..(188): 5′ITR
  misc_feature (192)..(1032): CAG(CBA)
  enhancer (194)..(436): CMV enhancer
  promoter (439)..(720): CB promoter
  Intron (720)..(1127): Hybrid Intron in CAG
  Intron (720)..(950): b-actin intron
  misc_feature (1158)..(1163): Kozak
  CDS (1164)..(3437): human Mfn2 engineered
  misc_feature (1379)..(1399): miR538 mut
  misc_feature (2534)..(2554): miR1518 mutated
  misc_feature (3438)..(3519): linker p4271
  misc_feature (3520)..(3547): miR538 AS
  misc_feature (3569)..(3587): loop
  misc_feature (3588)..(3606): miR538 sense
  misc_feature (3607)..(3651): 3′ flank
  misc_feature (3678)..(4266): WRPE
  misc_feature (4355)..(4474): SV40 PA temrinator
  repeat_region (4584)..(4771): 3′ITR
gctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagagtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatcccgtacgacgcgtgctagcaccggtgccaccatgagcctgctgttcagccggtgcaacagcatcgtgaccgtgaagaaaaacaagcggcacatggccgaagtgaacgcctctccactgaagcacttcgtgaccgccaagaagaagatcaacggcatcttcgagcagctgggcgcctacatccaagagagcgccaccttcctggaagatacctacagaaacgccgagctggaccccgtgaccacagaggaacaggtgctggatgtgaagggctacctgagcaaagtgcggggcatctctgaagtgctggccagacggcatatgaaggtggcctttttcggccggaccagcaacggcaagagcaccgtgatcaatgccatgctgtgggacaaagtgctgccctctggaatcggccacaccaccaactgctttctgagagtggaaggcaccgacggccacgaggcatttctgctgacagagggctccgaggaaaagcggagcgccaagacagtgaaccagctggcccatgctctgcaccaggataagcagctgcatgccggaagcctggtgtccgtgatgtggcccaatagcaagtgccctctgctgaaggacgacctggtgctgatggatagccccgggatcgatgtgaccaccgaactggacagctggatcgacaagttctgcctggacgccgacgtgttcgtgctggtggccaatagcgagagcaccctgatgcagaccgagaagcactttttccacaaggtgtccgagcggctgagcagacccaatatctttatcctgaacaacagatgggacgccagcgccagcgagcccgagtatatggaagaagtgcggcggcagcatatggaacggtgcacatcctttctggtggacgagctgggcgtcgtggatagatctcaggccggcgacagaatcttctttgtgtccgccaaagaggtgctgaacgcccggattcagaaagcccagggaatgcctgaaggcggaggtgcactggccgagggatttcaagtgcggatgttcgagttccagaacttcgagcggagattcgaggagtgcatcagccagagcgccgtcaagaccaagttcgagcagcataccgtgcgggccaagcagattgccgaagccgtcagactgatcatggacagcctgcatatggccgccagagagcagcaggtctactgcgaggaaatgcgggaagagagacaggaccggctgaagttcatcgacaagcagctggaactgctggcccaggactacaagctgcggatcaagcagatcaccgaagaggtggaaagacaggtgtccaccgctatggccgaggaaatcagacggctgagcgtgctggtcgacgactaccagatggactttcacccctctccagtggtgctgaaagtctacaaaaacgagctgcaccggcacatcgaggaaggcctgggcagaaacatgagcgacagatgcagcaccgccatcaccaatagcctgcagaccatgcagcaggacatgatcgacggcctgaaacctctgctgcctgtgtccgtcagatcccagatcgatatgctggtgccccggcagtgctttagcctgaactacgacctgaactgcgacaagctgtgcgccgatttccaagaggacatcgagtttcacttcagcctcggctggacaatgctggtcaacagatttctgggccccaagaacagcagacgggccctgatgggctacaacgatcaggtgcagaggcccattcctctgacacccgccaatcctagcatgcctccactgcctcagggcagcctgacacaagaggaattcatggtgtctatggtcaccggcctggccagcctgaccagcagaacatctatgggcatcctggtcgtcggcggcgttgtgtggaaagctgttggctggcggctgatcgccctgagctttggcctgtatggcctgctgtacgtgtacgagagactgacctggaccaccaaggccaaagagcgggccttcaagcggcagtttgtggaacacgcctccgagaaactgcagctggtgatcagctacaccggcagcaattgcagccaccaggtgcagcaagagctgtccggaacattcgcccacctgtgtcagcaggtcgacgtgaccagagagaacctggaacaagagatcgccgccatgaataagaaaatcgaggtgctcgactccctgcagagcaaggccaagctgctgagaaacaaggccggctggctggacagcgagctgaatatgttcacccaccagtacctgcagcctagccgctaataaagcggccgcaagcttgcacccgtacctttgggagcgcgcgccctcgtcgtgtcgtgacgtcacccgttctgttggatccctggaggcttgctgaaggctgtatgctgttgacgtccagaacctgttctgttttggccactgactgacagaacaggctggacgtcaacaggacacaaggcctgttactagcactcacatggaacaaatggcccagatcttaagcttctcgagatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgcccttctgctacgtcccttcggccctcaatccagcggaccttccttcccgcggcctgctgccggctctgcggcctcttccgcgtcttcgccttcgccctcagacgagtcggatctccctttgggccgcctccccgcctgctgatcgccctgagcctggccctggtgaccaacagccaggtgcaattgctagagtcgactctagagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagc

SEQ ID NO: 70 - protein, 757 aa, Artificial Sequence; identical to SEQ ID NO: 2
  Synthetic Construct

SEQ ID NO: 71 - DNA, 4771 bp, Artificial Sequence
  Vector genome CAG.CI.hMfn2.GA.link.miR1518.WPRE .SV40
  repeat_region (1)..(188): ITR
  misc_feature (192)..(1032): CAG (CBA)
  enhancer (194)..(436): CMV enhancer
  promoter (439)..(720): CB Promoter
  Intron (720)..(1127): Hybrid intron in CAG
  Intron (720)..(950): b-actin intron
  misc_feature (1158)..(1163): Kozak
  CDS (1164)..(3437): human Mfn2 optimized
  misc_feature (1379)..(1399): miR538 mut
  misc_feature (2534)..(2554): miR 1518 mutated
  misc_feature (3438)..(3519): linker p4271
  misc_feature (3520)..(3547): 5′ flank
  misc_feature (3569)..(3587): loop
  misc_feature (3588)..(3606): rat miR 1518 sense
  misc_feature (3607)..(3651): 3′ flank
  misc_feature (3678)..(4266): WRPE
  misc_feature (4355)..(4474): SV40 PA terminator
  repeat_region (4584)..(4771): ITR
  misc_feature (35488)..(3568): rat miR1518 AS
gctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagagtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatcccgtacgacgcgtgctagcaccggtgccaccatgagcctgctgttcagccggtgcaacagcatcgtgaccgtgaagaaaaacaagcggcacatggccgaagtgaacgcctctccactgaagcacttcgtgaccgccaagaagaagatcaacggcatcttcgagcagctgggcgcctacatccaagagagcgccaccttcctggaagatacctacagaaacgccgagctggaccccgtgaccacagaggaacaggtgctggatgtgaagggctacctgagcaaagtgcggggcatctctgaagtgctggccagacggcatatgaaggtggcctttttcggccggaccagcaacggcaagagcaccgtgatcaatgccatgctgtgggacaaagtgctgccctctggaatcggccacaccaccaactgctttctgagagtggaaggcaccgacggccacgaggcatttctgctgacagagggctccgaggaaaagcggagcgccaagacagtgaaccagctggcccatgctctgcaccaggataagcagctgcatgccggaagcctggtgtccgtgatgtggcccaatagcaagtgccctctgctgaaggacgacctggtgctgatggatagccccgggatcgatgtgaccaccgaactggacagctggatcgacaagttctgcctggacgccgacgtgttcgtgctggtggccaatagcgagagcaccctgatgcagaccgagaagcactttttccacaaggtgtccgagcggctgagcagacccaatatctttatcctgaacaacagatgggacgccagcgccagcgagcccgagtatatggaagaagtgcggcggcagcatatggaacggtgcacatcctttctggtggacgagctgggcgtcgtggatagatctcaggccggcgacagaatcttctttgtgtccgccaaagaggtgctgaacgcccggattcagaaagcccagggaatgcctgaaggcggaggtgcactggccgagggatttcaagtgcggatgttcgagttccagaacttcgagcggagattcgaggagtgcatcagccagagcgccgtcaagaccaagttcgagcagcataccgtgcgggccaagcagattgccgaagccgtcagactgatcatggacagcctgcatatggccgccagagagcagcaggtctactgcgaggaaatgcgggaagagagacaggaccggctgaagttcatcgacaagcagctggaactgctggcccaggactacaagctgcggatcaagcagatcaccgaagaggtggaaagacaggtgtccaccgctatggccgaggaaatcagacggctgagcgtgctggtcgacgactaccagatggactttcacccctctccagtggtgctgaaagtctacaaaaacgagctgcaccggcacatcgaggaaggcctgggcagaaacatgagcgacagatgcagcaccgccatcaccaatagcctgcagaccatgcagcaggacatgatcgacggcctgaaacctctgctgcctgtgtccgtcagatcccagatcgatatgctggtgccccggcagtgctttagcctgaactacgacctgaactgcgacaagctgtgcgccgatttccaagaggacatcgagtttcacttcagcctcggctggacaatgctggtcaacagatttctgggccccaagaacagcagacgggccctgatgggctacaacgatcaggtgcagaggcccattcctctgacacccgccaatcctagcatgcctccactgcctcagggcagcctgacacaagaggaattcatggtgtctatggtcaccggcctggccagcctgaccagcagaacatctatgggcatcctggtcgtcggcggcgttgtgtggaaagctgttggctggcggctgatcgccctgagctttggcctgtatggcctgctgtacgtgtacgagagactgacctggaccaccaaggccaaagagcgggccttcaagcggcagtttgtggaacacgcctccgagaaactgcagctggtgatcagctacaccggcagcaattgcagccaccaggtgcagcaagagctgtccggaacattcgcccacctgtgtcagcaggtcgacgtgaccagagagaacctggaacaagagatcgccgccatgaataagaaaatcgaggtgctcgactccctgcagagcaaggccaagctgctgagaaacaaggccggctggctggacagcgagctgaatatgttcacccaccagtacctgcagcctagccgctaataaagcggccgcaagcttgcacccgtacctttgggagcgcgcgccctcgtcgtgtcgtgacgtcacccgttctgttggatccctggaggcttgctgaaggctgtatgctgttcttataaaccttgaggacagttttggccactgactgactgtcctcagtttataagaacaggacacaaggcctgttactagcactcacatggaacaaatggcccagatcttaagcttctcgagatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgcccttctgctacgtcccttcggccctcaatccagcggaccttccttcccgcggcctgctgccggctctgcggcctcttccgcgtcttcgccttcgccctcagacgagtcggatctccctttgggccgcctccccgcctgctgatcgccctgagcctggccctggtgaccaacagccaggtgcaattgctagagtcgactctagagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagc

SEQ ID NO: 72 - protein, 757 aa, Artificial Sequence; identical to SEQ ID NO: 2
  Synthetic Construct

SEQ ID NO: 73 - DNA, 4559 bp, Artificial Sequence
  Vector genome CAG.CI.hMfn2.GA.WPRE.SV40
  repeat_region (1)..(188): ITR
  misc_feature (192)..(1032): CAG (CBA)
  enhancer (194)..(1032): CMV enhancer
  promoter (439)..(720): CB promoter
  Intron (720)..(1127): Hybrid intron in CAG
  Intron (720)..(950): b-actin intron
  misc_feature (1158)..(1163): Kozak
  CDS (1164)..(3437): huamn Mfn2 engineered
  misc_feature (1379)..(1399): miR538 mut
  misc_feature (2534)..(2554): miR1518 mutated
  misc_feature (3466)..(4054): WRPE
  misc_feature (4143)..(4262): SV 40 PA temrinator
  rep_origin (4372)..(4559): ITR
gctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagagtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatcccgtacgacgcgtgctagcaccggtgccaccatgagcctgctgttcagccggtgcaacagcatcgtgaccgtgaagaaaaacaagcggcacatggccgaagtgaacgcctctccactgaagcacttcgtgaccgccaagaagaagatcaacggcatcttcgagcagctgggcgcctacatccaagagagcgccaccttcctggaagatacctacagaaacgccgagctggaccccgtgaccacagaggaacaggtgctggatgtgaagggctacctgagcaaagtgcggggcatctctgaagtgctggccagacggcatatgaaggtggcctttttcggccggaccagcaacggcaagagcaccgtgatcaatgccatgctgtgggacaaagtgctgccctctggaatcggccacaccaccaactgctttctgagagtggaaggcaccgacggccacgaggcatttctgctgacagagggctccgaggaaaagcggagcgccaagacagtgaaccagctggcccatgctctgcaccaggataagcagctgcatgccggaagcctggtgtccgtgatgtggcccaatagcaagtgccctctgctgaaggacgacctggtgctgatggatagccccgggatcgatgtgaccaccgaactggacagctggatcgacaagttctgcctggacgccgacgtgttcgtgctggtggccaatagcgagagcaccctgatgcagaccgagaagcactttttccacaaggtgtccgagcggctgagcagacccaatatctttatcctgaacaacagatgggacgccagcgccagcgagcccgagtatatggaagaagtgcggcggcagcatatggaacggtgcacatcctttctggtggacgagctgggcgtcgtggatagatctcaggccggcgacagaatcttctttgtgtccgccaaagaggtgctgaacgcccggattcagaaagcccagggaatgcctgaaggcggaggtgcactggccgagggatttcaagtgcggatgttcgagttccagaacttcgagcggagattcgaggagtgcatcagccagagcgccgtcaagaccaagttcgagcagcataccgtgcgggccaagcagattgccgaagccgtcagactgatcatggacagcctgcatatggccgccagagagcagcaggtctactgcgaggaaatgcgggaagagagacaggaccggctgaagttcatcgacaagcagctggaactgctggcccaggactacaagctgcggatcaagcagatcaccgaagaggtggaaagacaggtgtccaccgctatggccgaggaaatcagacggctgagcgtgctggtcgacgactaccagatggactttcacccctctccagtggtgctgaaagtctacaaaaacgagctgcaccggcacatcgaggaaggcctgggcagaaacatgagcgacagatgcagcaccgccatcaccaatagcctgcagaccatgcagcaggacatgatcgacggcctgaaacctctgctgcctgtgtccgtcagatcccagatcgatatgctggtgccccggcagtgctttagcctgaactacgacctgaactgcgacaagctgtgcgccgatttccaagaggacatcgagtttcacttcagcctcggctggacaatgctggtcaacagatttctgggccccaagaacagcagacgggccctgatgggctacaacgatcaggtgcagaggcccattcctctgacacccgccaatcctagcatgcctccactgcctcagggcagcctgacacaagaggaattcatggtgtctatggtcaccggcctggccagcctgaccagcagaacatctatgggcatcctggtcgtcggcggcgttgtgtggaaagctgttggctggcggctgatcgccctgagctttggcctgtatggcctgctgtacgtgtacgagagactgacctggaccaccaaggccaaagagcgggccttcaagcggcagtttgtggaacacgcctccgagaaactgcagctggtgatcagctacaccggcagcaattgcagccaccaggtgcagcaagagctgtccggaacattcgcccacctgtgtcagcaggtcgacgtgaccagagagaacctggaacaagagatcgccgccatgaataagaaaatcgaggtgctcgactccctgcagagcaaggccaagctgctgagaaacaaggccggctggctggacagcgagctgaatatgttcacccaccagtacctgcagcctagccgctaataatgtacttaagcttctcgagatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgcccttctgctacgtcccttcggccctcaatccagcggaccttccttcccgcggcctgctgccggctctgcggcctcttccgcgtcttcgccttcgccctcagacgagtcggatctccctttgggccgcctccccgcctgctgatcgccctgagcctggccctggtgaccaacagccaggtgcaattgctagagtcgactctagagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagc

SEQ ID NO: 74 - protein, 757 aa, Artificial Sequence; identical to SEQ ID NO: 2
  Synthetic Construct

SEQ ID NO: 75 - DNA, 4821 bp, Artificial Sequence
  Vector genome CB7.CI.hMfn2.GA.link.miR538.rBG
  repeat_region (1)..(130): ITR
  enhancer (259)..(562): CMV enhancer
  promoter (561)..(842): CB7 promoter
  Intron (935)..(1907): chicken beta-actin intron
  misc_feature (1953)..(1958): Kozak
  CDS (1959)..(4232): human Mfn2 engineered
  misc_feature (2174)..(2194): miR538 mut
  misc_feature (3329)..(3349): miR1518
  misc_feature (4233)..(4314): linker p5271
  misc_feature (4315)..(4342): 5′ flank
  misc_feature (4343)..(4363): miR538 AS
  misc_feature (4364)..(4382): loop
  misc_feature (4383)..(4401): miR538 Sense
  misc_feature (4402)..(4446): 3′ flank
  polyA_signal (4500)..(4626): rabbit globin poly A
  repeat_region (4692)..(4821): ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatctgatatcactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcacgcgtgctaggatcccgtacgacgcgtgctagcaccggtgccaccatgagcctgctgttcagccggtgcaacagcatcgtgaccgtgaagaaaaacaagcggcacatggccgaagtgaacgcctctccactgaagcacttcgtgaccgccaagaagaagatcaacggcatcttcgagcagctgggcgcctacatccaagagagcgccaccttcctggaagatacctacagaaacgccgagctggaccccgtgaccacagaggaacaggtgctggatgtgaagggctacctgagcaaagtgcggggcatctctgaagtgctggccagacggcatatgaaggtggcctttttcggccggaccagcaacggcaagagcaccgtgatcaatgccatgctgtgggacaaagtgctgccctctggaatcggccacaccaccaactgctttctgagagtggaaggcaccgacggccacgaggcatttctgctgacagagggctccgaggaaaagcggagcgccaagacagtgaaccagctggcccatgctctgcaccaggataagcagctgcatgccggaagcctggtgtccgtgatgtggcccaatagcaagtgccctctgctgaaggacgacctggtgctgatggatagccccgggatcgatgtgaccaccgaactggacagctggatcgacaagttctgcctggacgccgacgtgttcgtgctggtggccaatagcgagagcaccctgatgcagaccgagaagcactttttccacaaggtgtccgagcggctgagcagacccaatatctttatcctgaacaacagatgggacgccagcgccagcgagcccgagtatatggaagaagtgcggcggcagcatatggaacggtgcacatcctttctggtggacgagctgggcgtcgtggatagatctcaggccggcgacagaatcttctttgtgtccgccaaagaggtgctgaacgcccggattcagaaagcccagggaatgcctgaaggcggaggtgcactggccgagggatttcaagtgcggatgttcgagttccagaacttcgagcggagattcgaggagtgcatcagccagagcgccgtcaagaccaagttcgagcagcataccgtgcgggccaagcagattgccgaagccgtcagactgatcatggacagcctgcatatggccgccagagagcagcaggtctactgcgaggaaatgcgggaagagagacaggaccggctgaagttcatcgacaagcagctggaactgctggcccaggactacaagctgcggatcaagcagatcaccgaagaggtggaaagacaggtgtccaccgctatggccgaggaaatcagacggctgagcgtgctggtcgacgactaccagatggactttcacccctctccagtggtgctgaaagtctacaaaaacgagctgcaccggcacatcgaggaaggcctgggcagaaacatgagcgacagatgcagcaccgccatcaccaatagcctgcagaccatgcagcaggacatgatcgacggcctgaaacctctgctgcctgtgtccgtcagatcccagatcgatatgctggtgccccggcagtgctttagcctgaactacgacctgaactgcgacaagctgtgcgccgatttccaagaggacatcgagtttcacttcagcctcggctggacaatgctggtcaacagatttctgggccccaagaacagcagacgggccctgatgggctacaacgatcaggtgcagaggcccattcctctgacacccgccaatcctagcatgcctccactgcctcagggcagcctgacacaagaggaattcatggtgtctatggtcaccggcctggccagcctgaccagcagaacatctatgggcatcctggtcgtcggcggcgttgtgtggaaagctgttggctggcggctgatcgccctgagctttggcctgtatggcctgctgtacgtgtacgagagactgacctggaccaccaaggccaaagagcgggccttcaagcggcagtttgtggaacacgcctccgagaaactgcagctggtgatcagctacaccggcagcaattgcagccaccaggtgcagcaagagctgtccggaacattcgcccacctgtgtcagcaggtcgacgtgaccagagagaacctggaacaagagatcgccgccatgaataagaaaatcgaggtgctcgactccctgcagagcaaggccaagctgctgagaaacaaggccggctggctggacagcgagctgaatatgttcacccaccagtacctgcagcctagccgctaataaagcggccgcaagcttgcacccgtacctttgggagcgcgcgccctcgtcgtgtcgtgacgtcacccgttctgttggatccctggaggcttgctgaaggctgtatgctgttgacgtccagaacctgttctgttttggccactgactgacagaacaggctggacgtcaacaggacacaaggcctgttactagcactcacatggaacaaatggcccagatcagctttctagagtcgacccgggcggcggcggccgcctaaggctcgaggatctttttccctctgccaaaaattatggggacatcatgaagccccttgagcatctgacttctggctaataaaggaaatttattttcattgcaatagtgtgttggaattttttgtgtctctcactcggataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 76 - protein, 757 aa, Artificial Sequence; identical to SEQ ID NO: 2
  Synthetic Construct

SEQ ID NO: 77 - DNA, 4905 bp, Artificial Sequence
  Vector genome CB7.CI.hMfn2.GA.link.miR1518.rBG
  repeat_region (1)..(130): ITR
  enhancer (259)..(562): CMV enhancer
  promoter (561)..(842): CB7 promoter
  Intron (935)..(1907): chicken beta-actin intron
  misc_feature (1953)..(1958): Kozak
  CDS (1959)..(4232): human Mfn2 engineered
  misc_feature (2174)..(2194): miR538 mut
  misc_feature (3329)..(3349): miR 1518 mutated
  misc_feature (4233)..(4314): linker p4271
  misc_feature (4315)..(4342): 5′ flank
  misc_feature (4343)..(4363): rat miR1518 AS
  misc_feature (4364)..(4382): loop
  misc_feature (4383)..(4401): rat miR 1518 sense
  misc_feature (4402)..(4446): 3′Flank
  polyA_signal (4584)..(4710): rabbit globin polyA
  repeat_region (4776)..(4905): ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatctgatatcactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcacgcgtgctaggatcccgtacgacgcgtgctagcaccggtgccaccatgagcctgctgttcagccggtgcaacagcatcgtgaccgtgaagaaaaacaagcggcacatggccgaagtgaacgcctctccactgaagcacttcgtgaccgccaagaagaagatcaacggcatcttcgagcagctgggcgcctacatccaagagagcgccaccttcctggaagatacctacagaaacgccgagctggaccccgtgaccacagaggaacaggtgctggatgtgaagggctacctgagcaaagtgcggggcatctctgaagtgctggccagacggcatatgaaggtggcctttttcggccggaccagcaacggcaagagcaccgtgatcaatgccatgctgtgggacaaagtgctgccctctggaatcggccacaccaccaactgctttctgagagtggaaggcaccgacggccacgaggcatttctgctgacagagggctccgaggaaaagcggagcgccaagacagtgaaccagctggcccatgctctgcaccaggataagcagctgcatgccggaagcctggtgtccgtgatgtggcccaatagcaagtgccctctgctgaaggacgacctggtgctgatggatagccccgggatcgatgtgaccaccgaactggacagctggatcgacaagttctgcctggacgccgacgtgttcgtgctggtggccaatagcgagagcaccctgatgcagaccgagaagcactttttccacaaggtgtccgagcggctgagcagacccaatatctttatcctgaacaacagatgggacgccagcgccagcgagcccgagtatatggaagaagtgcggcggcagcatatggaacggtgcacatcctttctggtggacgagctgggcgtcgtggatagatctcaggccggcgacagaatcttctttgtgtccgccaaagaggtgctgaacgcccggattcagaaagcccagggaatgcctgaaggcggaggtgcactggccgagggatttcaagtgcggatgttcgagttccagaacttcgagcggagattcgaggagtgcatcagccagagcgccgtcaagaccaagttcgagcagcataccgtgcgggccaagcagattgccgaagccgtcagactgatcatggacagcctgcatatggccgccagagagcagcaggtctactgcgaggaaatgcgggaagagagacaggaccggctgaagttcatcgacaagcagctggaactgctggcccaggactacaagctgcggatcaagcagatcaccgaagaggtggaaagacaggtgtccaccgctatggccgaggaaatcagacggctgagcgtgctggtcgacgactaccagatggactttcacccctctccagtggtgctgaaagtctacaaaaacgagctgcaccggcacatcgaggaaggcctgggcagaaacatgagcgacagatgcagcaccgccatcaccaatagcctgcagaccatgcagcaggacatgatcgacggcctgaaacctctgctgcctgtgtccgtcagatcccagatcgatatgctggtgccccggcagtgctttagcctgaactacgacctgaactgcgacaagctgtgcgccgatttccaagaggacatcgagtttcacttcagcctcggctggacaatgctggtcaacagatttctgggccccaagaacagcagacgggccctgatgggctacaacgatcaggtgcagaggcccattcctctgacacccgccaatcctagcatgcctccactgcctcagggcagcctgacacaagaggaattcatggtgtctatggtcaccggcctggccagcctgaccagcagaacatctatgggcatcctggtcgtcggcggcgttgtgtggaaagctgttggctggcggctgatcgccctgagctttggcctgtatggcctgctgtacgtgtacgagagactgacctggaccaccaaggccaaagagcgggccttcaagcggcagtttgtggaacacgcctccgagaaactgcagctggtgatcagctacaccggcagcaattgcagccaccaggtgcagcaagagctgtccggaacattcgcccacctgtgtcagcaggtcgacgtgaccagagagaacctggaacaagagatcgccgccatgaataagaaaatcgaggtgctcgactccctgcagagcaaggccaagctgctgagaaacaaggccggctggctggacagcgagctgaatatgttcacccaccagtacctgcagcctagccgctaataaagcggccgcaagcttgcacccgtacctttgggagcgcgcgccctcgtcgtgtcgtgacgtcacccgttctgttggatccctggaggcttgctgaaggctgtatgctgttcttataaaccttgaggacagttttggccactgactgactgtcctcagtttataagaacaggacacaaggcctgttactagcactcacatggaacaaatggcccagatctagggtggggccacctgccggtaggtgtgcggtaggcttttctccgtcgcaggacgcagggttcgggcctagagcttatcgttaagctttctagagtcgacccgggcggcggcggccgcctaaggctcgaggatctttttccctctgccaaaaattatggggacatcatgaagccccttgagcatctgacttctggctaataaaggaaatttattttcattgcaatagtgtgttggaattttttgtgtctctcactcggataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 78 - protein, 757 aa, Artificial Sequence; identical to SEQ ID NO: 2
  Synthetic Construct

SEQ ID NO: 79 - DNA, 4565 bp, Artificial Sequence
  Vector genome CB7.CI.hMfn2.GA.rBG
  rep_origin (1)..(130): ITR
  enhancer (259)..(562): CMV enhancer
  promoter (561)..(842): CB7 promoter
  TATA_signal (815)..(818): TATA signal
  Intron (935)..(1907): Chicken beta-actin intron
  misc_feature (1940)..(1948): Kozak
  CDS (1946)..(4219): huamn Mfn2 engineered
  misc_feature (2161)..(2181): miR538 mut
  misc_feature (3316)..(3336): miR1518 mutated
  polyA_signal (4244)..(4370): rabbit globin polyA
  repeat_region (4436)..(4565): ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatctgatatcactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcacgcgtgctagcaaggatccaccggtcgccaccatgagcctgctgttcagccggtgcaacagcatcgtgaccgtgaagaaaaacaagcggcacatggccgaagtgaacgcctctccactgaagcacttcgtgaccgccaagaagaagatcaacggaatcttcgagcagctgggcgcctacatccaagagagcgccaccttcctggaagatacctacagaaacgccgagctggaccccgtgaccacagaggaacaggtgctggatgtgaagggctacctgagcaaagtgcggggcatctctgaagtgctggccagacggcatatgaaggtggcctttttcggccggaccagcaacggcaagagcaccgtgatcaatgccatgctgtgggacaaagtgctgccctctggaatcggccacaccaccaactgctttctgagagtggaaggcaccgacggccacgaggcatttctgctgacagagggctccgaggaaaagcggagcgccaagacagtgaaccagctggcccatgctctgcaccaggataagcagctgcatgccggaagcctggtgtccgtgatgtggcccaatagcaagtgccctctgctgaaggacgacctggtgctgatggatagccccgggatcgatgtgaccaccgaactggacagctggatcgacaagttctgcctggacgccgacgtgttcgtgctggtggccaatagcgagagcaccctgatgcagaccgagaagcactttttccacaaggtgtccgagcggctgagcagacccaatatctttatcctgaacaacagatgggacgccagcgccagcgagcccgagtatatggaagaagtgcggcggcagcatatggaacggtgcacatcctttctggtggacgagctgggcgtcgtggatagatctcaggccggcgacagaatcttctttgtgtccgccaaagaggtgctgaacgcccggattcagaaagcccagggaatgcctgaaggcggaggtgcactggccgagggatttcaagtgcggatgttcgagttccagaacttcgagcggagattcgaggagtgcatcagccagagcgccgtcaagaccaagttcgagcagcataccgtgcgggccaagcagattgccgaagccgtcagactgatcatggacagcctgcatatggccgccagagagcagcaggtctactgcgaggaaatgcgggaagagagacaggaccggctgaagttcatcgacaagcagctggaactgctggcccaggactacaagctgcggatcaagcagatcaccgaagaggtggaaagacaggtgtccaccgctatggccgaggaaatcagacggctgagcgtgctggtcgacgactaccagatggactttcacccctctccagtggtgctgaaagtctacaaaaacgagctgcaccggcacatcgaggaaggcctgggcagaaacatgagcgacagatgcagcaccgccatcaccaatagcctgcagaccatgcagcaggacatgatcgacggcctgaaacctctgctgcctgtgtccgtcagatcccagatcgatatgctggtgccccggcagtgctttagcctgaactacgacctgaactgcgacaagctgtgcgccgatttccaagaggacatcgagtttcacttcagcctcggctggacaatgctggtcaacagatttctgggccccaagaacagcagacgggccctgatgggctacaacgatcaggtgcagaggcccattcctctgacacccgccaatcctagcatgcctccactgcctcagggcagcctgacacaagaggaattcatggtgtctatggtcaccggcctggccagcctgaccagcagaacatctatgggcatcctggtcgtcggcggcgttgtgtggaaagctgttggctggcggctgatcgccctgagctttggcctgtatggcctgctgtacgtgtacgagagactgacctggaccaccaaggccaaagagcgggccttcaagcggcagtttgtggaacacgcctccgagaaactgcagctggtgatcagctacaccggcagcaattgcagccaccaggtgcagcaagagctgtccggaacattcgcccacctgtgtcagcaggtcgacgtgaccagagagaacctggaacaagagatcgccgccatgaataagaaaatcgaggtgctcgactccctgcagagcaaggccaagctgctgagaaacaaggccggctggctggacagcgagctgaatatgttcacccaccagtacctgcagcctagccgctaataaagcggccgcctaaggctcgaggatctttttccctctgccaaaaattatggggacatcatgaagccccttgagcatctgacttctggctaataaaggaaatttattttcattgcaatagtgtgttggaattttttgtgtctctcactcggataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 80 - protein, 757 aa, Artificial Sequence; identical to SEQ ID NO: 2
  Synthetic Construct

SEQ ID NO: 81 - DNA, 2211 bp, adeno-associated virus hu68
  CDS (1)..(2211): AAVhu68
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctaacgcttaatgatggaagccaagccgtgggtcgttcgtccttttactgcctggaatatttcccgtcgcaaatgctaagaacgggtaacaacttccagttcagctacgagtttgagaacgtacctttccatagcagctatgctcacagccaaagcctggaccgactcatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccagtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtttattctgaaccccgccccattggcaccagatacctgactcgtaatctgtaa

SEQ ID NO: 82 - protein, 736 aa, adeno-associated virus hu68
MAADGYLPDWLEDNLSEGIREWWALKPGAPQPKANQQHQDNARGLVLPGYKYLGPGNGLDKGEPVNEADAAALEHDKAYDQQLKAGDNPYLKYNHADAEFQERLKEDTSFGGNLGRAVFQAKKRLLEPLGLVEEAAKTAPGKKRPVEQSPQEPDSSVGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 83 - DNA, 2211 bp, adeno-associated virus hu68
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtatggatacctcaccctgaacgacggcagtcaggcggtgggccgctcatccttctactgcctggagtacttcccttcgcagatgctgaggactggcaacaacttccagttcagctacgagttcgagaacgtccctttccacagcagctacgcccacagccagagtttggaccgcttgatgaaccctctgatcgaccagtacctgtactacctgtcaaagacgatcaacggttctggccagaaccagcagacgctgaagttcagcgtggccgggcctagcaacatggccgtccagggcagaaactacatccctgggcccagctaccggcagcagagagtctcaaccactgtgactcagaacaacaacagtgagttcgcctggcctggcgccagctcttgggccctcaacggccgcaactcgctgatgaacccaggcccagccatggccagtcacaaggagggcgaggaccgtttcttccctttgtctggctctctgatcttcggcaagcaggggaccggcagagacaacgtggacgcggacaaggtcatgatcacgaacgaggaggagatcaagaccaccaaccctgtggcaaccgagtcctacggccaggtggcaaccaaccaccagagcgcccaggcacaggcgcagactggctgggtccagaaccaggggatcctgcctggcatggtgtggcaggaccgtgacgtgtacctgcagggccctatctgggcaaagatccctcacacggacggcaacttccacccttctcctctgatgggcggcttcggcatgaagcacccgcctcctcagatcctcatcaagaacactccggtcccggcagaccctccgacggccttcaacaaggacaagctgaactcattcatcactcagtactccactggccaggtcagcgtggagatcgagtgggagctgcagaaggagaacagcaagcgttggaacccagagatccagtacacttccaactactacaagtctaacaacgtggagttcgccgtcaacactgagggtgtgtacagtgagcctcgccctatcggcacccggtacctcacccgaaacttgtga

SEQ ID NO: 84 - DNA, 2229 bp, adeno-associated virus PHP.eB
  CDS (1)..(2229): AAV-PHP.eB
atggctgccgatggttatcttccagattggctcgaggacaaccttagtgaaggaattcgcgagtggtgggctttgaaacctggagcccctcaacccaaggcaaatcaacaacatcaagacaacgctagaggtcttgtgcttccgggttacaaataccttggacccggcaacggactcgacaagggggagccggtcaacgcagcagacgcggcggccctcgagcacgacaaagcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctctttaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctagaactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggcctctcacaaagaaggagaggaccgtttctttcctttgtctggatctttaatttttggcaaacaaggtactggcagagacaacgtggatgcggacaaagtcatgataaccaacgaagaagaaattaaaactactaacccggtagcaacggagtcctatggacaagtggccacaaaccaccagagtgatgggactttggcggtgccttttaaggcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggtcaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctg

SEQ ID NO: 85 - protein, 743 aa, adeno-associated virus PHP.eB
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 86 - DNA, 841 bp, Artificial Sequence
  CB7 Promoter with CMV enhancer
ccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctaga

SEQ ID NO: 87 - DNA, 841 bp, Artificial Sequence; identical to SEQ ID NO: 86
  CAG promoter

SEQ ID NO: 88 - DNA, 127 bp, Artificial Sequence
  rabbit beta-globin
gatctttttccctctgccaaaaattatggggacatcatgaagccccttgagcatctgacttctggctaataaaggaaatttattttcattgcaatagtgtgttggaattttttgtgtctctcactcg

SEQ ID NO: 89 - DNA, 59 bp, Artificial Sequence
  miR1538
ttgacgtccagaacctgttctgttttggccactgactgacagaacaggctggacgtcaa

SEQ ID NO: 90 - DNA, 82 bp, Artificial Sequence
  linker
taaagcggccgcaagcttgcacccgtacctttgggagcgcgcgccctcgtcgtgtcgtgacgtcacccgttctgttggatcc